(19) United States
(12) Patent Application Publication
(10) Pub. No.: US 2026/0120864 A1
(43) Pub. Date: Apr. 30, 2026

(54) METHODS AND SYSTEMS FOR GENE ALTERATION PREDICTION FROM PATHOLOGY SLIDE IMAGES

(71) Applicants: F. HOFFMAN-LA ROCHE AG, Basel (CH); GENENTECH, INC., South San Francisco, CA (US); VENTANA MEDICAL SYSTEMS, INC., Tucson, AZ (US); HOFFMANN-LA ROCHE INC., Little Falls, NJ (US); FOUNDATION MEDICINE, INC., Cambridge, MA (US)

(72) Inventors: Paolo Santiago OCAMPO, South San Francisco, CA (US); Bernhard STIMPEL, Basel (CH); Yao NIE, Tucson, AZ (US); Fahime SHEIKHZADEH, Tucson, AZ (US); Xiao LI, South San Francisco, CA (US); Przemyslaw SZOSTAK, Basel (CH); Prasanna PORWAL, Basel (CH); Faranak AGHAEI, Tucson, AZ (US); James PAO, Cambridge, MA (US); Nathanial EDDY, Cambridge, MA (US); Lee ALBACKER, Cambridge, MA (US); Daniel DUNCAN, Cambridge, MA (US); Mikayla BIGGS, Cambridge, MA (US)

(21) Appl. No.: 18/287,438

(22) PCT Filed: Apr. 19, 2022
(86) PCT No.: PCT/US2022/025438

§ 371 Date: Oct. 18, 2023

Related U.S. Application Data
(60) Provisional application No. 63/176,826, filed on Apr. 19, 2021.
(60) Provisional application No. 63/188,963, filed on May 14, 2021.
(60) Provisional application No. 63/239,287, filed on Aug. 31, 2021.

(51) Int. Cl. G16H 50/20; G06N 20/00; G16B 20/20; G16B 40/30; G16H 20/10; G16H 30/40; G16H 50/70

(52) U.S. Cl. CPC G16H 50/20; G06N 20/00; G16B 20/20; G16B 40/30; G16H 20/10; G16H 30/40; G16H 50/70

(57) ABSTRACT

Described herein are methods and systems for determining gene alteration states from pathology images. Also described are methods of selecting a treatment for a medical disease, and treating a patient in need thereof, by determining gene alteration states from pathology images. The disclosed methods and systems may also be used to investigate and identify biomarkers corresponding to gene alteration statues of interest.

### CROSS-REFERENCE TO RELATED APPLICATIONS

This application claims the priority benefit of U.S. Provisional Patent Application Ser. No. 63/176,826, filed on Apr. 19, 2021, of U.S. Provisional Patent Application Ser. No. 63/188,963, filed on May 14, 2021, and of U.S. Provisional Patent Application Ser. No. 63/239,287, filed on Aug. 31, 2021, the contents of each of which are incorporated herein by reference in their entirety.

### FIELD

The present disclosure relates generally to methods and systems for determining a gene alteration (e.g., a point mutation, insertion, deletion, or fusion) in a tissue sample using tissue images, and methods and systems for treating a patient based on a determined gene alteration in a patient tissue sample from the tissue images.

### INTRODUCTION

Molecular characterization of solid tumors can produce a wide range of clinically impactful information, including diagnostic, prognostic, and predictive information in order to provide patients with increasingly personalized care. For example, for lung adenocarcinoma patients, a common usage of molecular assays can be to determine the patient's eligibility for one of the many targeted therapies that have been approved.

Whole slide pathology images, often acquired during operational tissue extraction procedures for either specimen record-keeping or research purposes, are high-resolution (e.g., megapixel or gigapixel) images of tissue specimens that have been excised or biopsied with diagnostic and/or curative intent. Previous methods for predicting gene alterations from whole slide images rely on training a machine learning model using annotated pathology slide images of highly selected tissue samples that are fairly homogeneous in terms of tissue phenotypes (e.g., tissue morphology or tissue histology). See, for example, Coudray, et al. 2018, “Classification and mutation prediction from non-small cell lung cancer histopathology images using deep learning”, Nature Medicine 24:1559-1567. These methods thus do not perform well when using pathology images of heterogeneous real-world tissue samples for which paired normal tissue samples are unavailable.

The capability to determine altered gene states in tissue samples directly from pathology slide images would provide the potential to vastly improve turnaround-times for patients and healthcare providers in the case of certain key actionable gene alteration states (e.g., detection of EGFR/ALK mutations in non-small-cell lung carcinoma (NSCLC) tissue samples, or detection of gene fusions/rearrangements in lung adenocarcinoma tissue sample to guide the choice of treatment by chemotherapy or a targeted therapy). Accordingly, there is a need for improved methods and systems for predicting gene alteration states from pathology slide images with greater accuracy and efficiency.

### BRIEF SUMMARY OF THE INVENTION

Described herein are methods and systems for more accurately determining gene alteration states in heterogeneous, real world tissue samples using a machine learning-based analysis of pathology images. In some embodiments, the disclosed methods can include the use of two machine learning models, one trained as a tissue classifier to process image patch data (extracted from pathology whole slide images of a tissue sample) in order to classify and label them according to tissue phenotype. The second machine learning model is trained as a gene alteration state classifier that processes the labeled image patch data produced by the first model and outputs a prediction of a gene alteration state (e.g., a mutation in one or more genes) exhibited by the tissue sample. In some instances, a third, front-end machine learning model may be used to extract image features from the image patch data and cluster them according to the similarity of their extracted features. In some instances, the latter approach enables classification of image patch data according to tissue phenotypes that may or may not be correlated with those visually recognized by a trained pathologist.

In some instances, the improvements in accuracy of the disclosed methods and systems are derived through adopting image patch-level annotation rather than whole slide image-level annotation of the labeled training data used to train a tissue phenotype classification model, which in turn is used to generate labeled image patch data that is paired with gene alteration state data (obtained, for example, using genotyping or next generation sequencing (NGS) data) to train a gene alteration state classification model.

In some instances, the improvements in accuracy of the disclosed methods and systems are derived through the use of a front-end machine learning model (e.g., a pre-trained or unsupervised image feature extraction model) used to extract image features from image patch data, which is then clustered and annotated by cluster type (e.g., annotated by a pathologist, or alternatively, by simply assigning a cluster label to the image patches within a given cluster), which in turn is used to generate labeled image patch data that is paired with gene alteration state data (obtained, for example, using genotyping or next generation sequencing (NGS) data) to train a gene alteration state classification model.

In some instances, the disclosed methods and systems may be used for the detection of oncogenic gene fusions in samples from a subject (e.g., a patient) based on an analysis of digital pathology images, such as scanned, stained (e.g., H&E stained) whole slide images of tissue specimens that include tumorous cells (e.g., lung adenocarcinoma). The disclosed methods for detection of actionable gene fusions may be based on one or more of: (i) automatic detection of histologic features, (ii) identification of mutually exclusive gene mutations (thereby ruling out the presence of a gene fusion), (iii) detection of NTRK gene fusions by grouping NTRK with ALK, ROS1, and RET into a single “actionable gene fusion cluster” and identifying the cluster, (iv) automatic detection of histologic features associated with ALK, ROS1, and RET (including solid and cribriform growth patterns, extracellular mucin, signet ring cells, goblet cells, and hepatoid cells), (v) identification and elimination of smoking-related mutational signatures, (vi) identification of low tumor mutation burden, (vii) identification of decreased per-slide tumor heterogeneity, (viii), identification and characterization of nuclear pleomorphism, or (ix) identification of pan-tumor or tumor-agnostic actionable gene fusion clusters using one or more end-to-end data-driven machine learning model(s).

Also described are methods of selecting a treatment for a medical disease, and treating a patient in need thereof, by determining gene alteration states from pathology images of tissue samples from the patient.

Described herein are methods for determining a gene alteration state in a tissue sample comprising: inputting, using one or more processors, a plurality of image patches derived from one or more pathology images of the tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; and outputting, using the one or more processors and the gene alteration state classification model, the gene alteration state for the tissue sample.

In some embodiments, the gene alteration state classification model is configured to determine a gene alteration state for one or more genes in the tissue sample based on the labeled image patch data. In some embodiments, the tissue phenotype classification model is trained using a plurality of tissue phenotype classification model training image patches, where each tissue phenotype classification model training image patch is labeled with a tissue phenotype class selected from a plurality of tissue phenotype classes. In some embodiments, the tissue phenotype classification model training image patches are derived from pathology images of tissue samples from the same tissue type. In some embodiments, the tissue phenotype classification model training image patches are manually labeled with the tissue phenotype class.

In some embodiments, the tissue phenotype classification model training image patches are labeled using a clustering process, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters.

In some embodiments, the image features are extracted from the tissue phenotype classification model training image patches using a pre-trained image feature extraction model. In some embodiments, the pre-trained image feature extraction model comprises an artificial neural network (ANN) model. The ANN model can be, for example, a convolutional neural network (CNN) model or any other ANN-based model.

In some embodiments, the image features are extracted from the tissue phenotype classification model training image patches using an unsupervised image feature extraction model. In some embodiments, the unsupervised image feature extraction model comprises an autoencoder model or a generative adversarial network (GAN) model.

In some embodiments, the tissue phenotype classification model training image patches are clustered using a k-means clustering method, hierarchical clustering method, a mixture model method, or any combination thereof.

In some embodiments, the method further comprises performing a dimensionality reduction on the extracted image features prior to clustering the tissue phenotype classification model training image patches based on a reduced representation of the extracted image features.

In some embodiments, the gene alteration state classification model is trained using a plurality of gene alteration state classification model training image patches, and wherein each gene alteration state classification model training image patch is labeled with a tissue phenotype class and a gene alteration state. In some embodiments, the gene alteration state determined for the tissue sample is a binary classification.

In some embodiments, the method further comprises displaying the determined gene alteration state on a display device. In some embodiments, the method further comprises generating a report of the determined gene alteration state. In some embodiments, the method further comprises transmitting a report of the determined gene alteration state to a healthcare provider through a computer network. In some embodiments, the method further comprises transmitting a report of the determined gene alteration state to a healthcare provider over the Internet.

In some embodiments, an output of the gene alteration state classification model is a determination of the presence or absence of a mutation in at least one gene in the tissue sample. In some embodiments, an output of the gene alteration state classification model is a probability that the tissue sample has a mutation in at least one gene. In some embodiments, an output of the gene alteration state classification model is a probability that the tissue sample does not have the mutation in at least one gene. In some embodiments, the determined gene alteration state corresponds to a point mutation, insertion, deletion, or any combination thereof, in a single gene. In some embodiments, the determined gene alteration state corresponds to a point mutation, insertion, deletion, or any combination thereof, in at least two genes.

In some embodiments, the plurality of tissue phenotype classes comprises one or more tumor phenotype classes, one or more normal phenotype classes, one or more stroma phenotype classes, one or more immune phenotype classes, one or more necrosis phenotype classes, or any combination thereof.

In some embodiments, the tissue phenotype classification model or the gene alteration state classification model is a neural network. In some embodiments, the tissue phenotype classification model and/or the gene alteration state classification model is an artificial neural network (ANN). The ANN model can be, for example, a convolutional neural network (CNN) model or any other ANN-based model.

In some embodiments, the one or more pathology images are images of a cancerous tissue sample. In some embodiments, the cancerous tissue sample is a lung cancer tissue sample. In some embodiments, the lung cancer tissue is lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma tissue.

In some embodiments, the gene alteration state comprises a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene or any combination thereof.

In some embodiments, the method further comprises obtaining the tissue sample from a subject. In some embodiments, the method further comprises preparing a pathology slide using the tissue sample. In some embodiments, the method further comprising imaging the pathology slide to acquire the one or more pathology images of the tissue sample. In some embodiments, the method further comprises pre-processing the one or more pathology images to eliminate non-tissue portions of the image and extract image patches.

In some embodiments, the one or more pathology images have been previously annotated. In some embodiments, the one or more pathology images have not been previously annotated. In some embodiments, the one or more pathology images are annotated by a supervised, a semi-supervised, or an unsupervised machine learning method.

Also disclosed herein are non-transitory computer-readable storage media comprising one or more computer program instructions for execution by one or more processors of a device, the one or more computer program instructions when executed by the one or more processors, cause the device to perform any of the methods disclosed herein.

Also disclosed herein are systems, comprising: one or more processors; a memory configured to store one or more computer program instructions, wherein the one or more computer program instructions, when executed by the one or more processors are configured to: input a plurality of image patches derived from one or more pathology images of a tissue sample into a tissue phenotype classification model, the tissue phenotype classification model configured to classify image patches into a tissue phenotype class; classify, using the tissue phenotype classification model, the image patches to generate a labeled image patch data set for the tissue sample; input the labeled image patch data set into a gene alteration state classification model, the gene alteration state classification model configured to determine a gene alteration state for the tissue sample based on the labeled image patch data set; and output, using the gene alteration state classification model, the gene alteration state for the tissue sample.

In some embodiments, the tissue phenotype classification model is trained using a plurality of tissue phenotype classification model training image patches, and where each tissue phenotype classification model training image patch is labeled with a tissue phenotype class selected from a plurality of tissue phenotype classes. In some embodiments, the tissue phenotype classification model training image patches are derived from pathology images of tissue samples from the same tissue type. In some embodiments, the tissue phenotype classification model training image patches are manually labeled with the tissue phenotype class. In some embodiments, the tissue phenotype classification model training image patches are labeled using a clustering process, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters.

In some embodiments, the image features are extracted from the tissue phenotype classification model training image patches using a pre-trained feature extraction model. In some embodiments, the pre-trained image feature extraction model comprises an artificial neural network (ANN). The ANN model can be, for example, a convolutional neural network (CNN) model or any other ANN-based model.

In some embodiments, the image features are extracted from the tissue phenotype classification model training image patches using an unsupervised image feature extraction model. In some embodiments, the unsupervised image feature extraction model comprises an autoencoder model or a generative adversarial network (GAN) model.

In some embodiments, the tissue phenotype classification model training image patches are clustered using a k means clustering method, a hierarchical clustering method, a mixture model method, or any combination thereof.

In some embodiments, the system functionality further comprises performing a dimensionality reduction on the extracted image features prior to clustering the tissue phenotype classification model training image patches based on a reduced representation of the extracted image features.

In some embodiments, the gene alteration state classification model is trained using a plurality of gene alteration state classification model training image patches, and wherein each gene alteration state classification model training image patch is labeled with a tissue phenotype class and a gene alteration state. In some embodiments, the gene alteration state determined for the tissue sample is a binary classification.

In some embodiments, the system functionality further comprises displaying the determined gene alteration state on a display device. In some embodiments, the system functionality further comprises generating a report of the determined gene alteration state. In some embodiments, the system functionality further comprises transmitting a report of the determined gene alteration state to a healthcare provider through a computer network. In some embodiments, the system functionality further comprises transmitting a report of the determined gene alteration state to a healthcare provider over the Internet.

In some embodiments, an output of the gene alteration state classification model is a determination of the presence or absence of a mutation in at least one gene in the tissue sample. In some embodiments, an output of the gene alteration state classification model is a probability that the tissue sample has a mutation in at least one gene. In some embodiments, an output of the gene alteration state classification model is a probability that the tissue sample does not have the mutation in at least one gene. In some embodiments, the determined gene alteration state corresponds to a point mutation, insertion, deletion, or any combination thereof, in a single gene. In some embodiments, the determined gene alteration state corresponds to a point mutation, insertion, deletion, or any combination thereof, in at least two genes.

In some embodiments, the plurality of tissue phenotype classes comprises one or more tumor phenotype classes, one or more normal phenotype classes, one or more stroma phenotype classes, one or more immune phenotype classes, one or more necrosis phenotype classes, or any combination thereof.

In some embodiments, the tissue phenotype classification model or the gene alteration state classification model is a neural network. In some embodiments, the tissue phenotype classification model or the gene alteration state classification model is an artificial neural network (ANN). The ANN model can be, for example, a convolutional neural network (CNN) model or any other ANN-based model.

In some embodiments, the one or more pathology images are images of a cancerous tissue sample. In some embodiments, the cancerous tissue sample is a lung cancer tissue sample. In some embodiments, the lung cancer tissue is lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma tissue.

In some embodiments, the gene alteration state comprises a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene or any combination thereof.

Disclosed herein are methods for selecting a treatment for an individual having cancer, the method comprising: determining a gene alteration state of a gene of interest in a tissue sample from the individual using any of the methods disclosed herein; and selecting a treatment based on the determined gene alteration state.

In some embodiments, the cancer is lung cancer. In some embodiments, the lung cancer is lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma.

In some embodiments, the gene alteration state comprises a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a RET oncogene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene or any combination thereof.

Also disclosed herein are methods of treating an individual having cancer comprising selecting a treatment for the individual using any of the methods disclosed herein; and administering the treatment to the individual. In some embodiments, the gene alteration state comprises a mutation in an epidermal growth factor receptor (EGFR) gene, and the selected treatment comprises a kinase inhibitor, a small molecule drug, an antibody or antibody fragment, or a cellular immunotherapy that inhibits EGFR activity. In some embodiments, the selected treatment comprises a kinase inhibitor, and the kinase inhibitor comprises a multi-specific kinase inhibitor, a specific kinase inhibitor, a specific tyrosine kinase inhibitor, a specific EGFR inhibitor, or a dual EGFR/ERBB inhibitor. In some embodiments, the gene alteration state comprises a mutation in an anaplastic lymphoma kinase (ALK) fusion oncogene, and the selected treatment comprises a specific kinase inhibitor that inhibits ALK activity. In some embodiments, the specific kinase inhibitor comprises one or more of crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684). In some embodiments, the gene alteration state comprises a mutation in a receptor tyrosine kinase (ROS1) oncogene, and the selected treatment comprises a specific kinase inhibitor that inhibits ROS1 activity. In some embodiments, the specific kinase inhibitor is entrectinib (RXDX-101, NMS-E628). In some embodiments, the gene alteration state comprises a mutation in a receptor tyrosine kinase (RET) oncogene, and the selected treatment comprises a specific kinase inhibitor that inhibits RET activity. In some embodiments, the specific kinase inhibitor is selpercatinib, pralsetinib, TPX-0046, or any combination thereof. In some embodiments, the gene alteration state comprises a mutation in a neurotrophic tyrosine receptor kinase (NTRK) oncogene, and the selected treatment comprises a specific NTRK inhibitor that inhibits NTRK activity. In some embodiments, the specific NTRK inhibitor comprises larotrectinib, entrectinib, LOXO-195, danusertib (PHA-739358), lestaurtinib, AZ-23, PHA-848125, CEP-2563, K252a, KRC-108, or any combination thereof. In some embodiments, the gene alteration state comprises a mutation in a homologous recombination repair (HRR) pathway gene, and the selected treatment comprises a platinum-based chemotherapy or a poly-ADP ribose polymerase (PARP) inhibitor that inhibits the activity of a mutated HRR pathway protein. In some embodiments, the poly-ADP ribose polymerase (PARP) inhibitor comprises olaparib, niraparib, rucaparib, or any combination thereof.

Disclosed herein are methods for determining a gene alteration state in a tissue sample comprising: inputting, using one or more processors, a plurality of image patches derived from one or more pathology images of the tissue sample into a tissue phenotype classification model, the tissue phenotype classification model configured to classify image patches into a tissue phenotype class; and classifying, using the one or more processors and the tissue phenotype classification model, the image patches to generate a labeled image patch data set corresponding to one or more tissue phenotype classes for the tissue sample by: extracting image features from the image patches of the plurality; clustering the image patches of the plurality based on the extracted image features; and labeling the image patches of the plurality based on the extracted image feature cluster to which they belong. In some embodiments, each of the one or more tissue phenotype classes corresponds to one or more extracted image feature clusters. In some embodiments, the method further comprises inputting, using the one or more processors, the labeled image patch data set into a gene alteration state classification model, the gene alteration state classification model configured to determine a gene alteration state for the tissue sample based on the labeled image patch data set; and outputting, using the one or more processors and the gene alteration state classification model, the gene alteration state for the tissue sample.

In some embodiments of any of the methods disclosed herein, the method may further comprise performing one or more additional procedures based on the determined gene alteration state. In some embodiments, the one or more additional procedures comprise one or more additional diagnostic tests. In some embodiments, the one or more additional diagnostic tests are used to confirm a diagnosis of disease. In some embodiments, the disease is cancer. In some embodiments, the one or more additional procedures comprise selecting, initiating, adjusting, or discontinuing a treatment for an individual having cancer. In some embodiments, the one or more additional procedures comprise treating an individual having cancer. In some embodiments, the one or more procedures comprise performing one or more genomic profiling assays. In some embodiments, the one or more genomic profiling assays are used to select a treatment for, or monitor progression of, a cancer in an individual having cancer. In some embodiments, performing the one or more genomic profiling assays comprises obtaining a nucleic acid sample from a patient from which the tissue sample was derived, and sequencing the nucleic acid to perform a molecular profiling test. In some embodiments, the nucleic acid sample comprises a deoxyribonucleic acid (DNA) sample or a ribonucleic acid (RNA) samples. In some embodiments, the deoxyribonucleic acid (DNA) sample comprises tissue-derived DNA, cell-free DNA (cfDNA), circulating tumor DNA (ctDNA), or mitochondrial DNA. In some embodiments, the ribonucleic acid (RNA) sample comprises messenger RNA (mRNA), ribosomal RNA (rRNA), transfer RNA (tRNA), or mitochondrial RNA. In some embodiments, the nucleic acid sample is derived from a tissue sample, a blood sample, a urine sample, a saliva sample, a biopsy sample, or a liquid biopsy sample from the patient. In some embodiments, the molecular profiling test comprises a comprehensive genomic profiling (CGP) test, a gene expression profiling test, a cancer hotspot panel test, a DNA methylation test, a DNA fragmentation test, an RNA fragmentation test, or any combination thereof. In some embodiments, a result of the molecular profiling teat is used for selecting, initiating, adjusting, or discontinuing a treatment for an individual having cancer. In some embodiments, the method further comprises treating the individual having cancer. In some embodiments, the method further comprises displaying a result of the molecular profiling test on a display device. In some embodiments, the method further comprises generating a report for the molecular profiling test. In some embodiments, the method further comprises transmitting a report for the molecular profiling test to a healthcare provider through a computer network. In some embodiments, the method further comprises transmitting a report for the molecular profiling test to a healthcare provider over the Internet. In some embodiments, the one or more additional procedures comprises performing a follow-up screening test. In some embodiments, the follow-up screening test comprises a colonoscopy. In some embodiments, the follow-up colonoscopy is performed on a more frequently interval as a result of the determined gene alteration state.

Disclosed herein are computer-implemented methods comprising: accessing a digital pathology image that depicts a particular section of a biological sample from a subject, and wherein the depicted particular section was stained with one or more stains; segmenting the digital pathology image into a plurality of image patches; classifying each of the plurality of image patches based on a determination that the image patch includes a depiction of a tumor region or a tumor nest structure; classifying the digital pathology image based on a weighted combination of the labels generated for each image patch of the digital pathology image based on a determination that the digital pathology image includes a depiction of an occurrence of gene fusion with respect to depicted oncological cells; generating a subject prediction from the digital pathology image based on the classification of the digital pathology image, wherein the subject prediction corresponds to a prediction of applicability of one or more treatment regimens for the subject based on the occurrence of gene fusion with response to the depicted oncological cells; and outputting the subject prediction.

In some embodiments, the computer-implemented method further comprising classifying the digital pathology image based on a determination that the digital pathology image includes a depiction of one or more mutations that are mutually exclusive with the occurrence of gene fusion or applicability of one or more treatment regimens. In some embodiments, the computer-implemented method further comprises classifying each of the plurality of image patches based on a tumor region morphology, the tumor region morphology corresponding to an analysis of one or more signet cells, one or more hepatoid cells, extracellular mucin, tumor mutational burden, tumor growth patterns, or tumor heterogeneity depicted in the region. In some embodiments, the computer-implemented method further comprises training one or more machine learning models to classify each of the plurality of image patches, wherein the one or more machine learning models are trained based on a set of training data comprising one or more labeled depictions of a tumor region or tumor nest structure and one or more labeled depictions not including a tumor region or tumor nest structure. In some embodiments, the subject prediction is further generated based on combining one or more second classifications of one or more second digital pathology images, each of the one or more second digital pathology images depicting a second particular sample of the biological sample from the subject. In some embodiments, outputting the subject prediction comprises outputting a graphical representation of the digital pathology image comprising an indication of the label generated for each image patch of the digital pathology image and a predicted level of confidence for the label for each image patch of the digital pathology image. In some embodiments, outputting the subject prediction comprises outputting a recommendation associated with use of the one or more treatment regimens.

Also disclosed are methods comprising: transmitting, from a client computing system to a remote computing system, a request communication to process a digital pathology image that depicts a particular section of a biological sample from a subject, wherein, in response to receiving the request communication from the client computing system, the remote computing system, performs operations comprising: accessing the digital pathology image that depicts the particular section of the biological sample from the subject, and wherein the depicted particular section was stained with one or more stains; segmenting the digital pathology image into a plurality of image patches; classifying each of the plurality of image patches based on a determination that the image patch includes a depiction of a tumor region or a tumor nest structure; classifying the digital pathology image based on a weighted combination of the labels generated for each image patch of the digital pathology image based on a determination that the digital pathology image includes a depiction of an occurrence of gene fusion with respect to depicted oncological cells; generating a subject prediction from the digital pathology image based on the classification of the digital pathology image, wherein the subject prediction corresponds to a prediction of applicability of one or more treatment regimens for the subject based on the occurrence of gene fusion with response to the depicted oncological cells; providing the subject prediction to the client computing system via a response communication; and outputting, by the client computing system in response to receiving the response communication, the subject prediction.

Disclosed herein are systems comprising: one or more data processors; and a computer-readable non-transitory storage medium including instructions that, when executed by the one or more data processors, cause the one or more data processors to perform part or all of one or more methods disclosed herein.

Disclosed herein are computer-program products tangibly embodied in a computer-readable non-transitory storage medium including instructions configured to cause one or more data processors to perform part or all of one or more methods disclosed herein.

Also disclosed herein are methods comprising, by a digital pathology image processing system: accessing a digital pathology image that depicts cancer cells in a particular section of a biological sample from a subject; segmenting the digital pathology image into a plurality of image patches; generating, for each of the plurality of image patches, a label indicating whether the image patch depicts a tumor region or a tumor nest structure; determining, based on the labels generated for each image patch, that the digital pathology image comprises a depiction of an occurrence of gene fusion with respect to the cancer cells; and generating, based on the occurrence of gene fusion with respect to the cancer cells, a subject prediction for the subject, wherein the subject prediction comprises a prediction of applicability of one or more treatment regimens for the subject.

In some embodiments, the method further comprises detecting one or more features from each of the plurality of image patches, wherein the one or more features comprise one or more of a clinical feature or a histologic feature, and wherein generating the label for each of the plurality of image patches is based on the one or more features. In some embodiments, generating the label for each of the plurality of image patches is based on tumor morphology, wherein the tumor morphology is based on an analysis of one or more of a presence of signet ring cells, a number of signet ring cells, a presence of hepatoid cells, a number of hepatoid cells, extracellular mucin, a tumor growth pattern, or tumor heterogeneity. In some embodiments, generating the label for each of the plurality of image patches is based on one or more machine-learning models, wherein the method further comprises training the one or more machine-learning models based on a plurality of training data comprising one or more labeled depictions of a tumor region or tumor nest structure and one or more labeled depictions of other histologic or clinical features. In some embodiments, the subject prediction is generated further based on an analysis of one or more additional digital pathology images, each of the one or more additional digital pathology images depicting an additional particular sample of the biological sample from the subject, and wherein the analysis comprises: determining whether each of the one or more additional digital pathology images comprises a depiction of an occurrence of gene fusion with respect to the cancer cells; and combining the determination for each of the one or more additional digital pathology images. In some embodiments, the method further comprises outputting, via a graphical user interface, the subject prediction, wherein the graphical user interface comprises a graphical representation of the digital pathology image, and wherein the graphical representation comprises an indication of the label generated for each of the plurality of image patches and a predicted level of confidence associated with the respective label. In some embodiments, the method further comprises generating a recommendation associated with use of the one or more treatment regimens. In some embodiments, the particular section of the biological sample was stained with one or more stains. In some embodiments, determining that the digital pathology image comprises the depiction of the occurrence of gene fusion with respect to the cancer cells is further based on a weighted combination of the labels generated for each image patch. In some embodiments, the method further comprises: identifying nuclear pleomorphism from the digital pathology image; and measuring the identified nuclear pleomorphism, wherein determining that the digital pathology image comprises the depiction of the occurrence of gene fusion is further based on the measured nuclear pleomorphism.

Disclosed herein are one or more computer-readable non-transitory storage media embodying software that is operable when executed to: access a digital pathology image that depicts cancer cells in a particular section of a biological sample from a subject; segment the digital pathology image into a plurality of image patches; generate, for each of the plurality of image patches, a label indicating whether the image patch depicts a tumor region or a tumor nest structure; determine, based on the labels generated for each image patch, that the digital pathology image comprises a depiction of an occurrence of gene fusion with respect to the cancer cells; and generate, based on the occurrence of gene fusion with respect to the cancer cells, a subject prediction for the subject, wherein the subject prediction comprises a prediction of applicability of one or more treatment regimens for the subject.

In some embodiments, the software is further operable when executed to: detect one or more features from each of the plurality of image patches, wherein the one or more features comprise one or more of a clinical feature, a histologic feature, or a cell type, and wherein generating the label for each of the plurality of image patches is based on the one or more features. In some embodiments, generating the label for each of the plurality of image patches is based on tumor morphology, wherein the tumor morphology is based on an analysis of one or more of a signet ring cell, a hepatoid cell, extracellular mucin, tumor mutational burden, a tumor growth pattern, or tumor heterogeneity. In some embodiments, generating the label for each of the plurality of image patches is based on one or more machine-learning models, wherein the method further comprises training the one or more machine-learning models based on a plurality of training data comprising one or more labeled depictions of a tumor region or tumor nest structure and one or more labeled depictions of not including a tumor region or tumor nest structure.

Disclosed herein are systems comprising: one or more processors; and a non-transitory memory coupled to the processors comprising instructions executable by the processors, the processors operable when executing the instructions to: access a digital pathology image that depicts cancer cells in a particular section of a biological sample from a subject; segment the digital pathology image into a plurality of image patches; generate, for each of the plurality of image patches, a label indicating whether the image patch depicts a tumor region or a tumor nest structure; determine, based on the labels generated for each image patch, that the digital pathology image comprises a depiction of an occurrence of gene fusion with respect to the cancer cells; and generate, based on the occurrence of gene fusion with respect to the cancer cells, a subject prediction for the subject, wherein the subject prediction comprises a prediction of applicability of one or more treatment regimens for the subject.

In some embodiments, the processors are further operable when executing the instructions to detect one or more features from each of the plurality of image patches, wherein the one or more features comprise one or more of a clinical feature, a histologic feature, or a cell type, and wherein generating the label for each of the plurality of image patches is based on the one or more features. In some embodiments, generating the label for each of the plurality of image patches is based on tumor morphology, wherein the tumor morphology is based on an analysis of one or more of a signet ring cell, a hepatoid cell, extracellular mucin, tumor mutational burden, a tumor growth pattern, or tumor heterogeneity. In some embodiments, generating the label for each of the plurality of image patches is based on one or more machine-learning models, wherein the method further comprises training the one or more machine-learning models based on a plurality of training data comprising one or more labeled depictions of a tumor region or tumor nest structure and one or more labeled depictions of not including a tumor region or tumor nest structure.

Disclosed herein are methods comprising: transmitting, from a client computing system to a remote computing system, a request communication to process a digital pathology image that depicts cancer cells in a particular section of a biological sample from a subject, wherein in response to receiving the request communication from the client computing system, the remote computing system performs operations comprising: accessing the digital pathology image; segmenting the digital pathology image into a plurality of image patches; generating, for each of the plurality of image patches, a label indicating whether the image patch depicts a tumor region or a tumor nest structure; determining, based on the labels generated for each image patch, that the digital pathology image comprises a depiction of an occurrence of gene fusion with respect to the cancer cells; generating, based on the occurrence of gene fusion with respect to the cancer cells, a subject prediction for the subject, wherein the subject prediction comprises a prediction of applicability of one or more treatment regimens for the subject; providing the subject prediction to the client computing system via a response communication; and outputting, by the client computing system in response to receiving the response communication, the subject prediction.

Also disclosed are methods comprising, by a digital pathology image processing system: accessing a digital pathology image that depicts cancer cells in a particular section of a biological sample from a subject; determining that the digital pathology image comprises a depiction of one or more mutations that are mutually exclusive with an occurrence of gene fusion; determining an absence of gene fusion with respect to the cancer cells; and generate, based on the absence of gene fusion with respect to the cancer cells, a subject prediction for the subject, wherein the subject prediction comprises a prediction of applicability of one or more treatment regimens for the subject.

Disclosed herein are methods for characterizing one or more pathology images of a tissue sample, the methods comprising: inputting, using one or more processors, a plurality of image patches derived from one or more pathology images of the tissue sample into a tissue phenotype classification model, the tissue phenotype classification model configured to classify image patches into a tissue phenotype class; and classifying, using the one or more processors and the tissue phenotype classification model, the image patches to generate a labeled image patch data set that characterizes the tissue sample based on: (i) extracting image features from the image patches of the plurality of image patches using an image feature extraction model; (ii) assigning an image patch of the plurality of image patches to an associated image patch cluster based on the extracted image features; and (iii) labeling the image patches of the plurality of image patches based on an associated image patch cluster to which they belong.

In some embodiments, a tissue phenotype class corresponds to one or more image patch clusters, and wherein each image patch cluster is associated with an extracted image feature cluster. In some embodiments, the image feature extraction model comprises an unsupervised image feature extraction model. In some embodiments, the extracted image features comprise latent image features that are not directly visible in the one or more pathology images. In some embodiments, the method further comprises inputting, using the one or more processors, the labeled image patch data set into a gene alteration state classification model, the gene alteration state classification model configured to determine a gene alteration state for the tissue sample based on the labeled image patch data set; and outputting, using the one or more processors and the gene alteration state classification model, the gene alteration state for the tissue sample. In some embodiments, the tissue phenotype classification model is trained using a plurality of tissue phenotype classification model training image patches, and wherein each tissue phenotype classification model training image patch is labeled with a tissue phenotype class selected from a plurality of tissue phenotype classes. In some embodiments, the tissue phenotype classification model training image patches are labeled using a clustering process to generate the associated image patch clusters, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters. In some embodiments, the gene alteration state classification model is trained using a plurality of gene alteration state classification model training image patches, and wherein each gene alteration state classification model training image patch is labeled with a tissue phenotype class and a gene alteration state. In some embodiments, the determined gene alteration state corresponds to a point mutation, insertion, deletion, copy number variation (CNV), rearrangement, fusion, homologous recombination deficiency mutation, or any combination thereof, in one or more genes.

Also disclosed herein are methods for determining a gene alteration state in a tissue sample comprising: inputting, using one or more processors, a plurality of image patches derived from one or more pathology images of the tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state in the tissue sample based on the labeled image patch data set; and outputting, using the one or more processors and the gene alteration state classification model, the gene alteration state for the one or more genes in the tissue sample.

In some embodiments, the gene alteration state classification model is configured to determine a gene alteration state for one or more genes in the tissue sample. In some embodiments, the gene alteration state classification model is configured to determine a gene alteration state for a genetic signature comprising mutations in a plurality of genes in the tissue sample. In some embodiments, the method further comprises generating or transmitting a report of the determined gene alteration state to a healthcare provider. In some embodiments, the tissue phenotype classification model is trained using a plurality of tissue phenotype classification model training image patches, and wherein each tissue phenotype classification model training image patch is labeled with a tissue phenotype class selected from a plurality of tissue phenotype classes. In some embodiments, the tissue phenotype classification model training image patches are labeled using a clustering process, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters. In some embodiments, the image features are extracted from the tissue phenotype classification model training image patches using a pre-trained image feature extraction model or an unsupervised image feature extraction model. In some embodiments, the gene alteration state classification model is trained using a plurality of gene alteration state classification model training image patches, and wherein each gene alteration state classification model training image patch is labeled with a tissue phenotype class and a gene alteration state. In some embodiments, an output of the gene alteration state classification model is a determination of the presence or absence of a mutation in at least one gene in the tissue sample. In some embodiments, an output of the gene alteration state classification model is a probability that the tissue sample has a mutation in at least one gene. In some embodiments, the determined gene alteration state corresponds to a point mutation, insertion, deletion, copy number variation (CNV), rearrangement, fusion, homologous recombination deficiency mutation, or any combination thereof, in the one or more genes. In some embodiments, the plurality of tissue phenotype classes comprises one or more tumor phenotype classes, one or more normal phenotype classes, one or more stroma phenotype classes, one or more immune phenotype classes, one or more necrosis phenotype classes, or any combination thereof. In some embodiments, the one or more pathology images are images of a cancerous tissue sample. In some embodiments, the cancerous tissue sample is a lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma tissue sample. In some embodiments, the gene alteration state comprises a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene, or any combination thereof. In some embodiments, the method further comprises performing one or more additional diagnostic test procedures based on the determined gene alteration state.

Disclosed herein are methods for selecting a treatment for an individual having cancer, the method comprising: determining a gene alteration state of a gene of interest in a tissue sample from the individual using any of the methods described herein; and selecting a treatment based on the determined gene alteration state.

In some embodiments, the cancer is lung cancer. In some embodiments, the cancer is lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma. In some embodiments, the method further comprising administering the treatment to the individual. In some embodiments, the gene alteration state comprises a mutation in an epidermal growth factor receptor (EGFR) gene, and the selected treatment comprises a kinase inhibitor, a small molecule drug, an antibody or antibody fragment, or a cellular immunotherapy that inhibits EGFR activity. In some embodiments, the selected treatment comprises a kinase inhibitor, and the kinase inhibitor comprises a multi-specific kinase inhibitor, a specific kinase inhibitor, a specific tyrosine kinase inhibitor, a specific EGFR inhibitor, or a dual EGFR/ERBB inhibitor.

In some embodiments, the gene alteration state comprises a mutation in an anaplastic lymphoma kinase (ALK) fusion oncogene, and the selected treatment comprises a specific kinase inhibitor that inhibits ALK activity. In some embodiments, the specific kinase inhibitor comprises one or more of crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684).

In some embodiments, the gene alteration state comprises a mutation in a receptor tyrosine kinase (ROS1) oncogene, and the selected treatment comprises a specific kinase inhibitor that inhibits ROS1 activity. In some embodiments, the specific kinase inhibitor is entrectinib (RXDX-101, NMS-E628).

In some embodiments, the gene alteration state comprises a mutation in a receptor tyrosine kinase (RET) oncogene, and the selected treatment comprises a specific kinase inhibitor that inhibits RET activity. In some embodiments, the specific kinase inhibitor is selpercatinib, pralsetinib, TPX-0046, or any combination thereof.

In some embodiments, the gene alteration state comprises a mutation in a neurotrophic tyrosine receptor kinase (NTRK) oncogene, and the selected treatment comprises a specific NTRK inhibitor that inhibits NTRK activity. In some embodiments, the specific NTRK inhibitor comprises larotrectinib, entrectinib, LOXO-195, danusertib (PHA-739358), lestaurtinib, AZ-23, PHA-848125, CEP-2563, K252a, KRC-108, or any combination thereof.

In some embodiments, the gene alteration state comprises a mutation in a homologous recombination repair (HRR) pathway gene, and the selected treatment comprises a platinum-based chemotherapy or a poly-ADP ribose polymerase (PARP) inhibitor that inhibits an activity of a mutated HRR pathway protein. In some embodiments, the poly-ADP ribose polymerase (PARP) inhibitor comprises olaparib, niraparib, rucaparib, or any combination thereof.

Disclosed herein are non-transitory computer-readable storage media comprising one or more computer program instructions for execution by one or more processors of a device, the one or more computer program instructions when executed by the one or more processors, cause the device to perform any of the methods described herein.

Disclosed herein are systems, comprising: one or more processors; and a memory configured to store one or more computer program instructions, wherein the one or more computer program instructions, when executed by the one or more processors are configured to cause the system to perform any of the methods described herein.

Also disclosed herein are methods comprising: inputting, using one or more processors, a plurality of image patches derived from one or more pathology images of a tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; and based on the gene alteration state of the tissue sample, determining, using the one or more processors and the gene alteration state classification model, whether a solid biopsy-based assay can be used to analyze the tissue sample.

In some embodiments, when a determination is made that the solid biopsy-based assay is capable of analyzing the tissue sample, the method further comprises performing the solid biopsy-based assay of the tissue sample. In some embodiments, when a determination is made that the solid biopsy-based assay is not capable of analyzing the tissue sample, reflexing to a liquid biopsy-based assay, the method further comprising: obtaining a liquid biopsy sample from an individual; and performing a liquid biopsy-based assay on the liquid biopsy sample. In some embodiments, the tissue sample and the liquid biopsy sample are obtained from the same individual. In some embodiments, the liquid biopsy sample comprises blood, plasma, cerebrospinal fluid, sputum, stool, urine, or saliva. In some embodiments, the liquid biopsy sample comprises circulating tumor cells (CTCs). In some embodiments, the liquid biopsy sample comprises cell-free DNA (cfDNA), circulating tumor DNA (ctDNA), or any combination thereof. In some embodiments, the solid biopsy-based assay or liquid biopsy-based assay comprises: providing a plurality of nucleic acid molecules extracted from a solid biopsy or liquid biopsy sample obtained from the same individual that the tissue sample was obtained from; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; and detecting, using the one or more processors, a gene alteration state in the solid biopsy or liquid biopsy sample based on the sequence read data. In some embodiments, one or more of the plurality of sequencing reads overlap one or more gene loci within a subgenomic interval in the solid biopsy or liquid biopsy sample. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous solid biopsy sample, and the non-tumor nucleic acid molecules are derived from a normal portion of the heterogeneous solid biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer. In some embodiments, the method further comprises generating, by the one or more processors, a report indicating the presence or absence of a gene alteration state in the solid biopsy or liquid biopsy sample. In some embodiments, the method further comprises transmitting the report to a healthcare provider. In some embodiments, the report is transmitted via a computer network or a peer-to-peer connection. In some embodiments, the gene alteration state classification model is configured to determine a gene alteration state for one or more genes in the tissue sample based on the labeled image patch data. In some embodiments, the tissue phenotype classification model is trained using a plurality of tissue phenotype classification model training image patches, and wherein each tissue phenotype classification model training image patch is labeled with a tissue phenotype class selected from a plurality of tissue phenotype classes. In some embodiments, the tissue phenotype classification model training image patches are manually labeled with the tissue phenotype class. In some embodiments, the tissue phenotype classification model training image patches are labeled using a clustering process, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters. In some embodiments, the image features are extracted from the tissue phenotype classification model training image patches using a pre-trained image feature extraction model. In some embodiments, the image features are extracted from the tissue phenotype classification model training image patches using an unsupervised image feature extraction model. In some embodiments, the method further comprises performing a dimensionality reduction on the extracted image features prior to clustering the tissue phenotype classification model training image patches based on a reduced representation of the extracted image features. In some embodiments, the gene alteration state classification model is trained using a plurality of gene alteration state classification model training image patches, and wherein each gene alteration state classification model training image patch is labeled with a tissue phenotype class and a gene alteration state. In some embodiments, an output of the gene alteration state classification model is a determination of the presence or absence of a mutation in at least one gene in the tissue sample. In some embodiments, an output of the gene alteration state classification model is a probability that the tissue sample has a mutation in at least one gene. In some embodiments, an output of the gene alteration state classification model is a probability that the tissue sample does not have a mutation in at least one gene. In some embodiments, the determined gene alteration state corresponds to a point mutation, insertion, deletion, copy number variation (CNV), rearrangement, fusion, homologous recombination deficiency mutation, or any combination thereof, in at least one gene. In some embodiments, the plurality of tissue phenotype classes comprises one or more tumor phenotype classes, one or more normal phenotype classes, one or more stroma phenotype classes, one or more immune phenotype classes, one or more necrosis phenotype classes, or any combination thereof. In some embodiments, the tissue phenotype classification model or the gene alteration state classification model is a neural network. In some embodiments, the one or more pathology images are images of a cancerous tissue sample.

Disclosed herein are methods, comprising: inputting, using one or more processors, a plurality of image patches derived from one or more pathology images of the tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; and based on the gene alteration of the tissue sample, predicting, using the one or more processors, an outcome of a solid-based biopsy assay of the tissue sample.

Also disclosed are methods of identifying one or more treatment options for an individual having a disease, the method comprising: inputting, using one or more processors, a plurality of image patches derived from one or more pathology images of the tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; and based on the gene alteration of the tissue sample, determining, using the one or more processors, one or more treatment option for treating the disease.

Disclosed herein are methods of treating an individual having a disease, the method comprising: inputting, using one or more processors, a plurality of image patches derived from one or more pathology images of a tissue sample from the individual into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; determining, using the one or more processors, a disease type of the disease of the individual based on the gene alteration state determined for the tissue sample; based on the determined disease type, determining, using the one or more processors, a therapy for treating the disease type; and based on the determined therapy, treating the individual with the therapy.

In some embodiments of any of the method disclosed herein the method may further comprising performing a confirmatory genomic profiling assay on a sample obtained from the individual following the determination of a gene alteration state based on the one or more pathology images. In some embodiments, the sample obtained from the individual comprises a solid biopsy sample or a liquid biopsy sample. In some embodiments, the sample comprises a liquid biopsy sample, and the liquid biopsy sample comprises blood, plasma, cerebrospinal fluid, sputum, stool, urine, or saliva. In some embodiments, the liquid biopsy sample comprises circulating tumor cells (CTCs). In some embodiments, the liquid biopsy sample comprises cell-free DNA (cfDNA), circulating tumor DNA (ctDNA), or any combination thereof. In some embodiments, the genomic profiling assay comprises: providing a plurality of nucleic acid molecules extracted from the solid biopsy or liquid biopsy sample; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; and detecting, using the one or more processors, a gene alteration state in the solid biopsy or liquid biopsy sample based on the sequence read data. In some embodiments, one or more of the plurality of sequencing reads overlap one or more gene loci within a subgenomic interval in the solid biopsy or liquid biopsy sample. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous solid biopsy sample, and the non-tumor nucleic acid molecules are derived from a normal portion of the heterogeneous solid biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer. In some embodiments, the method further comprises generating, by the one or more processors, a report indicating the presence or absence of a gene alteration state in the solid biopsy or liquid biopsy sample. In some embodiments, the method further comprises transmitting the report to a healthcare provider. In some embodiments, the report is transmitted via a computer network or a peer-to-peer connection. In some embodiments, the gene alteration state classification model is configured to determine a gene alteration state for one or more genes in the tissue sample based on the labeled image patch data. In some embodiments, the tissue phenotype classification model is trained using a plurality of tissue phenotype classification model training image patches, and wherein each tissue phenotype classification model training image patch is labeled with a tissue phenotype class selected from a plurality of tissue phenotype classes. In some embodiments, the tissue phenotype classification model training image patches are manually labeled with the tissue phenotype class. In some embodiments, the tissue phenotype classification model training image patches are labeled using a clustering process, the clustering process comprising extracting image features from the tissue phenotype classification model training image patches, and clustering the tissue phenotype classification model training image patches based on the extracted image features. In some embodiments, labels are assigned to the tissue phenotype classification model training image patches based on the extracted image feature clusters. In some embodiments, the image features are extracted from the tissue phenotype classification model training image patches using a pre-trained image feature extraction model. In some embodiments, the image features are extracted from the tissue phenotype classification model training image patches using an unsupervised image feature extraction model. In some embodiments, the method further comprises performing a dimensionality reduction on the extracted image features prior to clustering the tissue phenotype classification model training image patches based on a reduced representation of the extracted image features. In some embodiments, the gene alteration state classification model is trained using a plurality of gene alteration state classification model training image patches, and wherein each gene alteration state classification model training image patch is labeled with a tissue phenotype class and a gene alteration state. In some embodiments, an output of the gene alteration state classification model is a determination of the presence or absence of a mutation in at least one gene in the tissue sample. In some embodiments, an output of the gene alteration state classification model is a probability that the tissue sample has a mutation in at least one gene. In some embodiments, an output of the gene alteration state classification model is a probability that the tissue sample does not have a mutation in at least one gene. In some embodiments, the determined gene alteration state corresponds to a point mutation, insertion, deletion, copy number variation (CNV), rearrangement, fusion, homologous recombination deficiency mutation, or any combination thereof, in at least one gene. In some embodiments, the plurality of tissue phenotype classes comprises one or more tumor phenotype classes, one or more normal phenotype classes, one or more stroma phenotype classes, one or more immune phenotype classes, one or more necrosis phenotype classes, or any combination thereof. In some embodiments, the tissue phenotype classification model or the gene alteration state classification model is a neural network. In some embodiments, the one or more pathology images are images of a cancerous tissue sample. In some embodiments, the therapy comprises chemotherapy, radiation therapy, immunotherapy, a targeted therapy, or surgery.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term as used herein controls.

### BRIEF DESCRIPTION OF THE DRAWINGS

Various aspects of the disclosed methods and systems are set forth with particularity in the appended claims. A better understanding of the features and advantages of the disclosed methods and systems will be obtained by reference to the following detailed description of illustrative embodiments and the accompanying drawings, of which:

FIG. 1 illustrates a non-limiting example of a network of interacting computer systems that can be used for digital pathology image generation and processing, as described herein according to some embodiments.

FIG. 2 provides a schematic illustration of an exemplary process for the training of a tissue phenotype classification model and a downstream gene alteration state classification model for determining gene alteration states in tissue samples using a machine learning-based analysis of pathology images.

FIG. 3 provides a schematic illustration of an exemplary process for the training of a front-end image feature extraction model, a tissue phenotype classification model and a downstream gene alteration state classification model for determining gene alteration states in tissue samples using a machine learning-based analysis of pathology images.

FIG. 4 provides a schematic illustration of an exemplary process for detecting gene alteration states, e.g., gene fusions.

FIG. 5A provides a non-limiting example of tumor heterogeneity in uterine leiomyosarcoma.

FIG. 5B provides a non-limiting example of tumor heterogeneity in dermatofibrosarcoma protuberans.

FIG. 6 provides an exemplary workflow diagram for detecting gene fusions.

FIG. 7 provides a schematic illustration of an exemplary method for using a trained tissue phenotype classification model and a trained gene alteration state classification model for determining a gene alteration state for a tissue sample.

FIG. 8 provides a schematic illustration of an exemplary machine learning architecture comprising an artificial neural network with one hidden layer.

FIG. 9 provides a schematic illustration of an exemplary node within a layer of an artificial neural network or deep learning model architecture.

FIG. 10 provides a schematic illustration of an exemplary autoencoder.

FIG. 11 provides a non-limiting example of a computing system in accordance with one or more examples of the present disclosure.

FIG. 12 provides a non-limiting example of a process for training a tissue phenotype classifier and a gene alteration state classifier for determining gene alteration states in a tissue sample from pathology slide images.

FIG. 13 provides a non-limiting example of the output of the process illustrated in FIG. 12, with a tissue specimen image (left), a tissue morphology classification (tumor, normal, stroma, immune, necrosis) result (middle), and an image patch-level gene alteration state prediction result (right) obtained by training a gene alteration state classifier using only image patches of interest, e.g., tumor.

FIG. 14 provides non-limiting examples of tissue images and the corresponding expert annotations (labels) used to train an image patch classifier model to classify image patches extracted from pathology slide images according to tissue subtypes (e.g., tissue morphology phenotypes).

FIG. 15 provides a non-limiting example of a process for training a tissue phenotype classifier and a gene alteration state classifier for determining gene alteration states in a tissue sample from pathology slide images. The process illustrated in FIG. 15 uses an unsupervised machine learning model (e.g., an image feature extraction model) and clustering of image patches according to the extracted image features to generate the labeled image patch training data used to subsequently train a tissue phenotype classifier.

FIG. 16 provides a simplified illustration of a process for training a tissue phenotype classification model and a gene alteration state classification model for determining gene alteration states in a tissue sample from pathology slide images that uses an unsupervised feature extraction model as a front-end.

FIG. 17 provides a non-limiting example of images and image feature clusters from unsupervised learning on feature vectors from deep neural networks that have been pre-trained on the ImageNet dataset. Each row represents a different cluster and contains 10 examples of image patches whose latent features belong to that cluster. The pathology assessments of representative image patches in each cluster are listed below the images.

FIG. 18A provides a non-limiting example of pathology slide images for lung adenocarcinoma.

FIG. 18B provides a non-limiting example of results from quality control.

FIG. 18C provides a non-limiting example of tumor region detection.

FIG. 18D provides a non-limiting example prediction of gene fusion status.

FIG. 19 provides a non-limiting example prediction of ROS1 gene fusion status.

FIG. 20 provides a non-limiting example of an ROC curve for image patch-based fusion prediction.

FIG. 21 provides a schematic illustration of a process for enabling end users to request subject predictions.

FIG. 22 provides a schematic illustration of a process for ruling out gene fusion.

### DETAILED DESCRIPTION

Described herein are methods and systems for more accurately determining gene alteration states in heterogeneous, real-world tissue samples using a machine learning-based analysis of pathology images. The disclosed methods can include the use of two machine learning models, one trained as a tissue classifier to process image patch data (extracted from pathology whole slide images of a tissue sample) in order to classify and label them according to tissue phenotype. The second machine learning model is trained as a gene alteration state classifier that processes the labeled image patch data produced by the first model and outputs a prediction of a gene alteration state (e.g., a mutation) exhibited by the tissue sample. In some instances, a third, front-end machine learning model may be used to extract image features from the image patch data and cluster them according to the similarity of their extracted features. In some instances, the latter approach enables classification of image patch data according to tissue phenotypes that may or may not be correlated with those visually recognized by a trained pathologist. Also described are methods of selecting a treatment for a medical disease, and treating a patient in need thereof, by determining gene alteration states from pathology images of tissue samples from the patient.

A method for determining a gene alteration state in a tissue sample according to the present disclosure can include inputting, using one or more processors, a plurality of image patches derived from one or more pathology images of the tissue sample into a tissue phenotype classification model, the tissue phenotype classification model configured to classify image patches into a tissue phenotype class; classifying, using the one or more processors and the tissue phenotype classification model, the image patches to generate a labeled image patch data set for the tissue sample; inputting, using the one or more processors, the labeled image patch data set into a gene alteration state classification model, the gene alteration state classification model configured to determine a gene alteration state for the tissue sample based on the labeled image patch data set; and outputting, using the one or more processors and the gene alteration state classification model, the gene alteration state for the tissue sample.

As noted above, the tissue phenotype classification model is a machine learning model trained to classify input image patches (derived from a tissue sample of unknown gene alteration state) according to tissue phenotype class and output labeled image patch data. Image patches (small sections of a whole slide pathology image comprising contiguous subsets of image pixels) are extracted from the whole slide image by, for example, masking the image to eliminate non-tissue regions and segmenting the remaining portions of the image to create contiguous subsets of image pixels. In some instances, a front-end image feature extraction model may be used to process image patch data and extract image features related to tissue phenotype class (e.g., tissue morphology, tissue histology, and the like), which may then be used to cluster image patches according to image features prior to annotation or labeling.

The gene alteration state classification model is a machine learning model trained to process the input labeled image patch data and output a determination of gene alteration state for the tissue sample (e.g., detection of a particular genetic mutation where a mutation may refer to a point mutation, insertion, deletion, copy number variation (CNV), or any combination thereof). In some instances, the gene alteration state classification model may be configured to determine a gene alteration state for one or more genes in the tissue sample. In some instances, the gene alteration state classification model may be configured to determine a gene alteration state for a genetic signature comprising mutations in a plurality of genes in the tissue sample. Any of a variety of supervised or unsupervised machine learning models known to those of skill in the art may be used for implementing the disclosed methods (e.g., an artificial neural network model (or deep learning model)). In some instances, the artificial neural network may be a convolutional neural network, as will be discussed in more detail below.

The disclosed methods and systems provide improvements in the accuracy for determining and outputting a gene alteration state for typically heterogeneous real world pathology tissue samples that are derived through: (i) the use of higher spatial resolution image annotation of tissue phenotype (e.g., at the image patch level rather than the whole slide image level), and/or (ii) the use of machine learning-based image feature extraction (from a plurality of image patches) and clustering to generate the labeled training data used to train a tissue phenotype classification model. The trained tissue phenotype classification model is then used (while in system training mode) to generate labeled image patch data that is paired with gene alteration state data (obtained, for example, using genotyping or next generation sequencing (NGS) data) to train a gene alteration state classification model. Once the system has been deployed, the trained tissue phenotype classification model is used in a pathology lab or other healthcare setting to process input image patch data derived from a pathology image of unknown gene alteration state and output tissue phenotype-labeled image patch data, which in turn is input into the trained gene alteration state classification model to determine and output a gene classification state for the tissue sample. The methods and systems may provide enhanced decision making insight to patients and/or healthcare providers via accelerated feedback on whether or not an actionable gene alteration state (e.g., a gene alteration state associated with a disease such as lung cancer) has been detected—well in advance of the physical sample being shipped to a genotyping or sequencing lab for confirmation of the determined gene alteration state.

Also disclosed herein are systems that include: one or more processors; a memory configured to store one or more computer program instructions, wherein the one or more computer program instructions, when executed by the one or more processors are configured to: input a plurality of image patches derived from one or more pathology images of a tissue sample into a tissue phenotype classification model, the tissue phenotype classification model configured to classify image patches into a tissue phenotype class; classify, using the tissue phenotype classification model, the image patches to generate a labeled image patch data set for the tissue sample; input the labeled image patch data set into a gene alteration state classification model, the gene alteration state classification model configured to determine a gene alteration state for the tissue sample based on the labeled image patch data set; and output, using the gene alteration state classification model, the gene alteration state for the tissue sample. The disclosed systems may be configured as local workstations, local computer systems, or distributed networks of computer systems or servers where, in some instances, all or a portion of the image data processing and analysis may optionally be performed in the cloud.

Also disclosed are non-transitory computer-readable storage media comprising one or more computer program instructions for execution by one or more processors of a device, the one or more computer program instructions that, when executed by the one or more processors, cause the device to perform any of the gene alteration state determination methods described.

Methods for selecting a treatment for an individual having cancer can include: determining a gene alteration state of a gene of interest in a tissue sample from the individual using any of the gene alteration state methods described herein; and selecting a treatment based on the determined gene alteration state. The disclosed methods also include methods of treating an individual having cancer comprising selecting a treatment for the individual using any of the gene alteration state methods described herein; and administering the treatment to the individual. In some instances, the cancer may comprise lung cancer. In some instances, the lung cancer may comprise lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, lung small cell carcinoma, or any combination thereof. In some instances, the gene alteration state that is detected may comprise a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a RET oncogene, a receptor tyrosine kinase (RET) oncogene, a neurotrophic tyrosine receptor kinase (NTRK) oncogene, or any combination thereof.

In some instances, the disclosed methods and systems may be applied to the detection of gene fusions/rearrangements, a specific type of rare, druggable oncogenic mutation event that can be identified across many different cancer types, that if present in a tumor tissue sample may indicate a robust response to certain targeted therapies. Gene fusions/rearrangements include rare and druggable mutation events that can occur across many different tumor types and are increasingly targeted by novel therapies. The identification of gene fusions can be a technically-difficult, expensive, and time-consuming process that may only benefit a minority of patients that carry such genetic alterations; for these reasons, widespread testing may be limited to those relatively few hospitals that can provide the technical and financial resources required. In some instances, the methods and systems described herein may address this disparity through the creation, training, and use of machine-learning models (e.g., digital pathology screening models) that can predict the presence of oncogenic fusions from digital pathology images such as scanned, stained (e.g., hematoxylin and eosin stained) whole slide images depicting cancer tissue/cells (e.g., lung adenocarcinoma). In addition, the methods and system disclosed herein may provide fast, cheap, and sufficiently-accurate screening tools that may be used to guide molecular testing and decision-making regarding the use of targeted therapies for individual patients (including, but not limited to, lung adenocarcinoma patients).

In some instances, a digital pathology image processing system of the present disclosure may access a digital pathology image that depicts cancer cells in a particular section of a biological sample from a subject. The digital pathology image processing system may then segment the digital pathology image into a plurality of image patches (also referred to herein as image tiles). In some cases, an image patch may include a portion of an image tile. An image patch may also include one or more image tiles or one or more portions of images tiles. The digital pathology image processing system may generate, for each of the plurality of image patches, a label indicating whether the image patch depicts, for example, a tumor region or a tumor nest structure. In some instances, the digital pathology image processing system may determine, based on the labels generated for each image patch, that the digital pathology image comprises a depiction of an occurrence of, e.g., a gene fusion, in the cancer cells present in the biological sample. The digital pathology image processing system may further generate, based on the detection of, e.g., a gene fusion, a subject prediction for the subject. In some instances, the subject prediction may comprise a prediction of the applicability of one or more treatment regimens (e.g., chemotherapy or a targeted therapy) for the subject.

### Definitions

The singular forms “a”, “an”, and “the” include plural references unless the context clearly dictates otherwise. Any reference to “or” herein is intended to encompass “and/or” unless otherwise stated.

The terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unnamed additives, components, integers, elements, or method steps.

The term “image feature” refers to a property of an image or image patch that contains information about the content of the image or image patch, e.g., image features may be specific structures in the image or image patch such as points, edges, shapes, textures, or objects, or may be non-visual or non-human-interpretable properties of the image derived from an image processing-and/or machine learning-based analysis of an image.

The term “tissue phenotype class” refers to a tissue morphology type, a tissue histology type, or any other tissue phenotype that is discernable upon visual inspection by a pathologist. The tissue phenotype class may be, but need not be, visually identifiable by a pathologist. For example, in some instances, tissue phenotype classes may be defined by clustered image feature categories that have been identified by a machine-learning model used to process pathology images of tissue samples.

The term “gene alteration state” refers to the presence or absence of a mutation in one or more genes in a tissue sample.

As used herein, the terms “classification model” and “classifier” are used interchangeably, and refer to a machine learning architecture or model that has been trained to sort input data into one or more labeled classes or categories.

As used herein, the term “binary output” refers to the instance where the output of a machine learning classifier sorts input data into one of two labeled classes or categories, e.g., a yes/no answer as to whether or not a given gene alteration state is present in a tissue sample.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease or condition, delay in recurrence of the disease or condition, or inhibition of the disease or condition. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

### Image-Based Detection of Altered Gene States

The disclosed methods utilize a machine learning-based approach to image processing for detection and determination of gene alteration states in a tissue sample. The methods may be applied to any tissue sample that is prepared for imaging in a pathology lab, for example, soft tissue samples (e.g., lung tissue surgical specimens or muscle biopsies) or solid tissue samples (e.g., bone marrow biopsies). Whole slide pathology images are processed to extract a plurality of image patches, which are then input into a trained tissue phenotype classification model configured to classify and label individual image patches according to tissue phenotype class (e.g., any of a variety of normal and/or abnormal tissue morphology and/or tissue histology classes known to those of skill in the art). The labeled image patch data for the tissue sample is then input into a trained gene alteration state classification model that is configured to determine and output a gene alteration state in the tissue sample. In some instances, the output of the gene alteration state classifier is binary, e.g., a yes/no determination of whether or not a particular genetic mutation is exhibited by the tissue sample. In some instances, the output of the gene alteration state classifier is non-binary, e.g., the output of the gene alteration state classifier may be a determination that one of several possible gene alteration states is exhibited by the tissue sample.

The improved accuracy of the disclosed methods for determining gene alteration state when working with real world pathology tissue samples, which are typically very heterogeneous in nature, is derived, in part, from the use of improved techniques for training the tissue classification model that classifies image patch data derived from the pathology whole slide image into one of a plurality of possible tissue phenotype classes. During the training phase, pathology whole slide images are selected to construct a tissue sample cohort of interest (e.g., tissue samples comprising a specific gene alteration state that has been previously determined using a genotyping assay or nucleic acid sequencing technique). Image patches are extracted from a subset of the whole slide images in the cohort and are optionally annotated by pathologists for tissue phenotype class. These annotated (also referred to herein as labeled) image patches are then used as a training data set to train a machine learning model (e.g., a convolutional neural network model) to classify non-labeled image patches extracted from pathology images of tissue samples as belonging to a particular tissue phenotype class. The trained tissue phenotype classification model may then be used to comprehensively classify image patches extracted from the remaining images in the cohort, thus generating tissue phenotype profiles (comprising labeled image patch data sets) for all relevant tissue samples.

In some instance, the training of the tissue phenotype classification model may comprise the use a machine learning approach to generate the labeled image patch training data (i.e., instead of the image patches being directly annotated by a pathologist). A cohort of relevant pathology slide images is constructed and image patches are extracted from the whole slide pathology images of the cohort as described above. Image feature extraction is then performed on image patches derived from a random subset of the pathology slide images using either a pre-trained neural network model or an unsupervised machine learning model to identify image patch features that are correlated with one or more tissue phenotype classes of interest. The set of image features extracted from the image patches, which may or may not be subject to additional image processing steps, are used to cluster the image patches by their salient features.

The use of machine learning-based image feature extraction and image patch clustering enables data processing that significantly improves downstream model training performance, including the training of tissue phenotype classification models and gene alteration state classification models. Examples of machine learning models that may be used for this step include either pre-trained machine learning models (e.g., the Inception V3 convolutional neural network trained on the ImageNet dataset) or unsupervised machine learning models (e.g., an autoencoder or generative adversarial network (GAN) that has been trained to model either the broad pathology visual distribution or a disease ontology-specific (e.g., lung adenocarcinoma) sub-distribution of tissue phenotypes) to extract features from image patches.

Specifically, if the image deconstruction (feature extraction) model chosen is a pre-trained network, then extracting features will entail passing the image patches through the network and using the calculated results (e.g., a vector of features) of some intermediate network layer (often the penultimate network layer) as an often non-human-interpretable encoding/compression of salient image features as calculated via a forward pass through the network. If a trained unsupervised model is used for image deconstruction, the encoding may take the form of some output from an intermediate layer of a model trained for a separate task of computer vision, such as from the discriminator network of a GAN tasked for binary prediction of data veracity. Use of autoencoders, another unsupervised method, would seek to encode data inputs into a latent embedding before decoding that embedding into a perfect reconstruction of the original data inputs. The encoder portion of a well-trained encoder network may then be used to directly distill input patches into a set of relevant salient features.

The reason for extracting image feature vectors from image patches is to enable effective clustering of the patches. Image patches cannot be effectively clustered using raw pixel values, but it has been shown that a significant reduction in dimensionality (as will be discussed below) and the distillation of salient information is possible using the aforementioned feature extraction methods. These reduced representations can be used to effectively cluster patch image groups across the pathology visual space. For example, in lung adenocarcinomas, labeled image patch clusters may be broad and could, for example, be characterized by their tissue morphology phenotypes, including tumor tissue, normal tissue, immune foci, necrotic regions, and stroma. In some instance, labeled image patch clusters may be even more specific, with the capability of distinguishing between tumor histological subtypes such as lepidic, acinar, micropapillary, papillary, solid, and mucinous subtypes.

In some instances, pathologists may then review the image patch clusters and assign labels according to the morphological and/or histological characteristics that they observe. In some instances, the image patch clusters may be assigned a label according to cluster type that is independent of interpretation by a trained pathologist. Once image patches extracted from a cohort of pathology images have been clustered and either annotated by a pathologist or assigned a cluster-based label, selected sets of labeled image patch data may be used to train a machine learning model, such as a convolutional neural network or other artificial neural network, to classify image patch data as belonging to specific tissue phenotype classes. This tissue phenotype classification model, trained using the labeled clusters of image patch data, can then be used to generate tissue phenotype class profiles for image patches extracted from the remaining images in the cohort. The trained tissue phenotype classification model allows tissue samples to be classified with respect to tissue phenotype class at an image patch-level of spatial resolution (or possibly at greater resolution with the use of advanced image acquisition and/or additional image processing steps).

In some instances, as part of the training phase, labeled image patches selected from a cohort of pathology images of interest (e.g., labeled image patches that have been classified as belonging to selected tissue phenotype classes and for which gene alteration states in the corresponding tissue samples are known from genotyping or nucleic acid sequencing results), may be iteratively used to investigate the presence of a signal or indicator for specific gene alteration states, i.e., to identify specific set(s) of labeled image patch data that are best correlated with a specific gene alteration state of interest. If the tissue specimens of interest are highly heterogeneous with respect to tissue phenotype class and/or highly variable in terms of gene alteration signal or indicator, the disclosed methods allow for more precise dataset refinement when training and tuning the tissue phenotype classification model and/or the downstream gene alteration state classification model. For example, in some instances, the tissue phenotype classification model and/or the gene alteration state classification model may be trained using only labeled image patches from the tissue phenotype class of interest (e.g., only labeled image patches correlated with tumor tissue, only labeled image patches correlated with inflammatory regions, etc.).

In the training phase, labeled image patch data generated by the trained tissue phenotype classification model, paired with corresponding gene alteration state data (e.g., genotyping and/or nucleic acid sequence data), is subsequently used to train another machine learning model (e.g., a convolutional neural network model or other artificial neural network) as a gene alteration state classification model to map the labeled image patch data for one or more tissue phenotype classes to one or more gene alteration states. The motivation for using an upstream tissue phenotype classification model is that all tissue phenotype classes are unlikely to contain equal signal that is indicative of a specific gene alteration state. For example, phenotypic changes that may provide indication of altered gene states are likely more detectable in tumor regions, and less likely in histological classes such as normal tissue. If the tissue specimens are highly heterogeneous but not all tissue phenotype classes provide a reliable indicator of altered gene state, then indiscriminately including all labeled tissue image patches for downstream model training is likely to introduce significant noise that may negatively impact the training and performance of a gene alteration state classification model. Using the front-end image deconstruction/feature extraction/image patch clustering approach to provide labeled image patch data for training a tissue phenotype classification model that in turn can characterize all of the pathology slide images in the training cohort enables one to select only the subset of image patches/regions belonging to relevant tissue phenotype classes for use in training the gene alteration state classification model.

In some instances, the gene alteration state classifier may determine (or detect) a gene alteration state classification for a tissue sample where the classification result output by the model is based on classification of the individual input image patches followed by aggregation of the individual image patch predictions using an aggregation method such as taking the average or median of the image patch predictions for each slide. In some instances, more complex gene alteration state classification models could use the aggregated image patch result and combine it with other features, such as the percentage of a selected tissue phenotype class present in a tissue sample (e.g., the percentage of image patches predicted as tumor) to make the final prediction.

In some instances, the disclosed methods and systems may use an alternative machine learning approach for training the gene alteration state classification model, such as a multiple-instance-learning (MIL) method. For example, the tissue phenotype classifier model may still be trained at an image patch level, where each patch has been assigned a label. Following training, the tissue phenotype classification model would be used to classify all tissue regions in the cohort, as described above. This would again allow the use only certain image patch groups of interest (i.e., those exhibiting the strongest signal for a given gene alteration state) for training the gene alteration classification model. The gene alteration classification model could then be trained using the MIL approach, where all or a subset of the image patches extracted from a slide are selected and passed through the MIL model. The MIL model would process all input image patches and aggregate the extracted information into a single output prediction for the slide (this in contrast to the image patch-level gene alteration classifier described above, which outputs a gene alteration state prediction for each image patch, which are then aggregated to generate a slide-level prediction). The single output prediction from the MIL model for a batch of image patches is compared to the slide label (e.g., an NGS result for gene alteration state) during training and validation of the MIL model.

Applicant has performed studies that demonstrate the need for a deliberate characterization and sub-selection of tissue image patches in training a tissue phenotype classification model. Studies utilizing slide-level labeling approaches (e.g., where every image region in a whole slide image inherits that slide's tissue phenotype and gene alteration state annotations) to train classifier models with cohort sizes equal to, or substantially greater than, those that have been publicly disclosed to date result in performance improvements that are barely appreciable over the performance of “no-skill” models (i.e., models that perform only as well as random guessing).

A deployed image-based gene alteration state classification system of the present disclosure would allow a user, e.g., a pathologist or pathology lab technician, to process one or more pathology images of a tissue sample and classify image patches extracted from the one or more pathology images by tissue phenotype class using a tissue classification model. A gene alteration state classification model, trained on the biomarker signals detected for specific tissue phenotype classes, would then be used to determine if a specific gene alteration state is exhibited in the tissue sample. The capability to determine altered gene states in tissue samples from pathology slide images would provide the potential to vastly improve turnaround-times for patients and healthcare providers when assessing certain key actionable gene alteration states (e.g., detection of EGFR/ALK mutations in non-small-cell lung carcinoma (NSCLC) to guide the choice of treatment by chemotherapy or a targeted therapy). The approach may also reduce or eliminate the requirements for follow-up confirmatory genotyping or nucleic acid sequencing data. The disclosed methods and systems may also be used to investigate or predict biomarkers and identify other gene alteration states of interest.

In some instances, for example, the disclosed methods and systems may be used to identify biomarkers comprising one or more alterations in one or more of ABL1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, C11orf30, C17orf39, CALR, CARD11, CASP8, CBFB, CBL, CCND1, CCND2, CCND3, CCNE1, CD22, CD70, CD74, CD79A, CD79B, CD274, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D, KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NSD3, NT5C2, NTRK1 NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK. TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WTI, XPO1, XRCC2, ZNF217, and ZNF703, or any combination thereof. In some instances, the biomarker comprises one or more alteration in PIK3CA. In some instances, of the one or more alterations comprise a base substitution, an insertion/deletion (indel), a gene fusion, a copy number alteration, or a genomic rearrangement.

In some instances, as noted elsewhere herein, the disclosed methods and systems may be used to identify gene fusions that are increasingly targeted by novel therapies. Targeted therapies for patients with tumors may include medicines that target epidermal growth factor receptor (EGFR), as well as the gene fusions involving anaplastic lymphoma kinase (ALK), RET, ROS1, and neurotrophic tyrosine receptor kinase (NTRK). For EGFR, although immunohistochemical stains can be used to identify the most common variants (e.g., with coverage of up to 97% of EGFR-positive lung adenocarcinoma patients), molecular testing may be required to identify resistance mutations in patients who have failed EGFR-targeted therapy. No such immunohistochemical stain has been developed for detection of RET and ROS1 variants, and the performance of the immunohistochemical stains for detection of ALK and NTRK variants may be highly variable and difficult to interpret. Furthermore, gene fusions often require more sophisticated molecular assays with greater coverage of the genome than the more commonly used “hot spot” assays that test for a limited number of loci. To target gene fusions, one may need a much wider coverage resulting in a much more expensive test, which requires much more technical capacity for a laboratory to perform. As a result, a significant proportion of patients may be unlikely to receive the correct test to determine whether their tumors carry gene fusions. Aside from that, some gene fusions (e.g., NTRK fusions) may be exceedingly rare. As an example, and not by way of limitation, although NTRK fusions have been identified in a wide variety of tumor types, the frequency of this specific fusion may be less than 1% in the most common cancer indications (such as in lung adenocarcinoma, colorectal cancer, and non-secretory breast cancer). The relative rarity of gene fusions (e.g., ranging from 7% for ALK to less than 0.3% for NTRK in lung adenocarcinomas) constitute a significant technical and financial disincentive to widespread testing. Indeed, studies have shown that the patient populations who benefit most from these drugs are those who live close to academic institutions who have the expertise, infrastructure, and budget to perform complex laboratory tests. Currently, molecular testing is the only method available to determine whether gene fusions exist in patients. However, molecular testing is expensive and patients sometimes avoid scheduling molecular testing due to the expense and/or unnecessary expense is incurred for patients who may not benefit from molecular testing. The methods and systems described herein present an improvement over current systems in that they may be used to identify patients that may benefit from molecular testing. In particular, the digital pathology image processing systems described herein may use a digital pathology machine learning model to screen for patients who are likely to have gene fusions, and may then provide a recommendation that those patients get tested using molecular assays. As a result, the disclosed digital pathology image processing systems may improve the likelihood of detecting gene fusions among patients, and may reduce the cost for follow-up molecular testing, thereby further benefiting and improving healthcare outcomes for those patients exhibiting gene fusions for which targeted therapies exist.

### Digital Pathology Image Generation and Processing

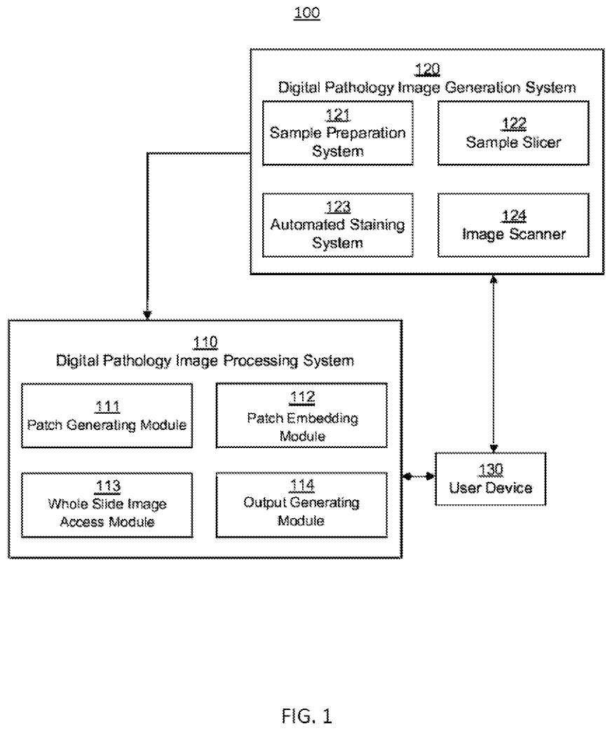

FIG. 1 illustrates a network 100 of interacting computer systems that can be used for digital pathology image generation and processing, as described herein according to some instances of the disclosed methods and systems.

A digital pathology image generation system 120 can generate one or more whole slide images or other related digital pathology images, corresponding to a particular sample. For example, an image generated by digital pathology image generation system 120 can include a stained section of a biopsy sample. As another example, an image generated by digital pathology image generation system 120 can include a slide image (e.g., a blood film) of a liquid sample. As another example, an image generated by digital pathology image generation system 120 can include fluorescence microscopy such as a slide image depicting fluorescence in situ hybridization (FISH) after a fluorescent probe has been bound to a target DNA or RNA sequence.

Some types of samples (e.g., biopsies, solid samples and/or samples including tissue) can be processed by a sample preparation system 121 to fix and/or embed the sample. Sample preparation system 121 can facilitate infiltrating the sample with a fixating agent (e.g., liquid fixing agent, such as a formaldehyde solution) and/or embedding substance (e.g., a histological wax). For example, a sample fixation sub-system can fix a sample by exposing the sample to a fixating agent for at least a threshold amount of time (e.g., at least 3 hours, at least 6 hours, or at least 13 hours). A dehydration sub-system can dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system can infiltrate the sample (e.g., one or more times for corresponding predefined time periods) with a heated (e.g., and thus liquid) histological wax. The histological wax can include a paraffin wax and potentially one or more resins (e.g., styrene or polyethylene). The sample and wax can then be cooled, and the wax-infiltrated sample can then be blocked out.

A sample slicer 122 can receive the fixed and embedded sample and can produce a set of sections. Sample slicer 122 can expose the fixed and embedded sample to cool or cold temperatures. Sample slicer 122 can then cut the chilled sample (or a trimmed version thereof) to produce a set of sections. Each section can have a thickness that is (for example) less than 100 μm, less than 50 μm, less than 10 μm or less than 5 μm. Each section can have a thickness that is (for example) greater than 0.1 μm, greater than 1 μm, greater than 2 μm or greater than 4 μm. The cutting of the chilled sample can be performed in a warm water bath (e.g., at a temperature of at least 30° C., at least 35° C. or at least 40° C.).

An automated staining system 123 can facilitate staining one or more of the sample sections by exposing each section to one or more staining agents. Each section can be exposed to a predefined volume of staining agent for a predefined period of time. In some instances, a single section is concurrently or sequentially exposed to multiple staining agents.

Each of one or more stained sections can be presented to an image scanner 124, which can capture a digital image of the section. Image scanner 124 can include a microscope camera. The image scanner 124 can capture the digital image at multiple levels of magnification (e.g., using a 10× objective, 20× objective, 40× objective, etc.). Manipulation of the image can be used to capture a selected portion of the sample at the desired range of magnifications. Image scanner 124 can further capture annotations and/or morphometrics identified by a human operator. In some instances, a section is returned to automated staining system 123 after one or more images are captured, such that the section can be washed, exposed to one or more other stains, and imaged again. When multiple stains are used, the stains can be selected to have different color profiles, such that a first region of an image corresponding to a first section portion that absorbed a large amount of a first stain can be distinguished from a second region of the image (or a different image) corresponding to a second section portion that absorbed a large amount of a second stain.

It will be appreciated that one or more components of digital pathology image generation system 120 can, in some instances, operate in connection with human operators. For example, human operators can move the sample across various sub-systems (e.g., of sample preparation system 121 or of digital pathology image generation system 120) and/or initiate or terminate operation of one or more sub-systems, systems, or components of digital pathology image generation system 120. As another example, part or all of one or more components of digital pathology image generation system (e.g., one or more subsystems of the sample preparation system 121) can be partly or entirely replaced with actions of a human operator.

Further, it will be appreciated that, while various described and depicted functions and components of digital pathology image generation system 120 pertain to processing of a solid and/or biopsy sample, other embodiments can relate to a liquid sample (e.g., a blood sample). For example, digital pathology image generation system 120 can receive a liquid-sample (e.g., blood or urine) slide, that includes a base slide, smeared liquid sample and cover. Image scanner 124 can then capture an image of the sample slide. Further embodiments of the digital pathology image generation system 120 can relate to capturing images of samples using advancing imaging techniques, such as FISH, described herein. For example, once a florescent probe has been introduced to a sample and allowed to bind to a target sequence appropriate imaging can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or one user devices 130 to submit one or more requests (e.g., that identify a subject) that a sample be processed by digital pathology image generation system 120 and that a resulting image be processed by a digital pathology image processing system 110.

Digital pathology image generation system 120 can transmit an image produced by image scanner 124 back to user device 130. User device 130 then communicates with the digital pathology image processing system 110 to initiate automated processing of the image. In some instances, digital pathology image generation system 120 provides an image produced by image scanner 124 to the digital pathology image processing system 110 directly, e.g. at the direction of the user of a user device 130. Although not illustrated, other intermediary devices (e.g., data stores of a server connected to the digital pathology image generation system 120 or digital pathology image processing system 110) can also be used. Additionally, for the sake of simplicity only one digital pathology image processing system 110, image generating system 120, and user device 130 is illustrated in the network 100. This disclosure anticipates the use of one or more of each type of system and component thereof without necessarily deviating from the teachings of this disclosure.

The network 100 and associated systems shown in FIG. 1 can be used in a variety of contexts where scanning and evaluation of digital pathology images, such as whole slide images, are an essential component of the work. As an example, the network 100 can be associated with a clinical environment, where a user is evaluating the sample for possible diagnostic purposes. The user can review the image using the user device 130 prior to providing the image to the digital pathology image processing system 110. The user can provide additional information to the digital pathology image processing system 110 that can be used to guide or direct the analysis of the image by the digital pathology image processing system 110. For example, the user can provide a prospective diagnosis or preliminary assessment of features within the scan. The user can also provide additional context, such as the type of tissue being reviewed. As another example, the network 100 can be associated with a laboratory environment were tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to the digital pathology image processing system 110.

Digital pathology image processing system 110 can process digital pathology images, including whole slide images, to classify the digital pathology images and generate annotations for the digital pathology images and related output. As an example, the digital pathology image processing system 110 can process whole slide images of tissue samples, or image patches (also referred to herein as image tiles) of the whole slide images of tissue samples generated by the digital pathology image processing system 110, to identify morphological traits, such as tumor regions or tumor nest structures (e.g., a cluster of tumor cells surrounded by tumor stroma), and determine occurrences of gene alteration events, such as gene fusions, based on the identified morphological traits such as tumor regions or tumor nest structures. The digital pathology image processing system 110 may use sliding windows to generate a mask over the tumor regions or tumor nest structures. In addition to its use for identifying, e.g., tumor regions or tumor nest structures in the whole slide image, the mask may be also used for measuring thickness, determining lengths for different endpoints, determining curviness for tortuosity, and measuring volume in a three-dimensional imaging or processing scenario. The digital pathology image processing system 110 may then crop the querying image into a plurality of image patches. A patch-generating module 111 can define a set of image patches for each digital pathology image. To define the set of patches, the patch-generating module 111 can segment the digital pathology image into the set of image patches. In some instances, the image patches can be non-overlapping (e.g., each patch includes pixels of the image not included in any other patch) or overlapping (e.g., each patch includes some portion of pixels of the image that are included in at least one other patch). Features such as whether or not image patches overlap, in addition to the size of each patch and the stride of the window (e.g., the image distance or number of pixels between an image patch and a subsequent patch) can increase or decrease the data set for analysis, with more image patches (e.g., achieved through the use of overlapping or smaller patches) increasing the potential resolution of eventual output and visualization. In some instances, patch-generating module 111 defines a set of image patches for an image where each patch is of a predefined size and/or an offset between patches is predefined. Continuing with the example of detecting gene fusions or other gene alterations, each pathology slide image may be cropped into image patches with a width and height of certain number of pixels. Furthermore, in some instances, the patch-generating module 111 can create multiple sets of image patches of varying size, overlap, step size, etc., for each whole slide image. As an example, in some instances, the width and height of each image patch (in terms of a number of pixels) may be dynamically determined (i.e., not fixed) based on factors such as the evaluation task at hand, the query image itself, or any other suitable factor. In some instances, the digital pathology image itself can contain image patch overlap, which may result from the imaging technique. In some instances, even segmentation performed without image patch overlap may be preferable to balance patch processing requirements and avoid influencing the embedding generation and weighting value generation discussed herein. An image patch size or patch offset can be determined, for example, by calculating one or more performance metrics (e.g., precision, recall, accuracy, and/or error) for each size/offset and by selecting a patch size and/or offset associated with one or more performance metrics above a predetermined threshold and/or associated with one or more performance metric(s) (e.g., high precision, high recall, high accuracy, and/or low error).

In some instances, the patch-generating module 111 may further define a patch size depending on the type of abnormality being detected. For example, the patch-generating module 111 can be configured to incorporate an awareness of the type(s) of tissue phenotypic traits or abnormalities that the digital pathology image processing system 110 will be searching for, and can customize the patch size according to the tissue phenotypes or abnormalities (and according to tissue sample type, in some instances) to improve detection. For example, the image generating module 111 can determine that, when the tissue phenotypes or abnormalities include searching for inflammation or necrosis in lung tissue, the patch size should be reduced to increase the scanning rate, while when the tissue abnormalities include abnormalities with Kupffer cells in liver tissues, the patch size should be increased to increase the opportunities for the digital pathology image processing system 110 to analyze the Kupffer cells holistically. In some instances, patch-generating module 111 may define a set of patches where a number of patches in the set, a size of the patches of the set, the resolution of the patches for the set, or other related properties, for each whole slide image is defined and held constant for each of one or more images.

In some instances, the patch-generating module 111 may further define the set of patches for each digital pathology image along one or more color channels or color combinations. As an example, digital pathology images received by digital pathology image processing system 110 can include large-format multi-color channel images having pixel color values (e.g., bit values corresponding to intensities) specified for each pixel of the image for one of several color channels. Example color specifications or color spaces that can be used include the RGB, CMYK, HSL, HSV, or HSB color specifications. The set of image patches can be defined based on segmenting the color channels and/or generating a brightness map or greyscale equivalent of each patch. For example, for each segment of an image, the patch-generating module 111 can provide a red patch, blue patch, green patch, and/or brightness patch, or the equivalent for the color specification used. As explained herein, segmenting the digital pathology images based on segments of the image and/or color values of the segments can improve the accuracy and recognition rates of the models/networks used to generate embeddings (e.g., lower-dimensional representations of image features) for the image patches and digital pathology image and to produce classifications of the digital pathology image. Additionally, the digital pathology image processing system 110, e.g., using patch-generating module 111, can convert between color specifications and/or prepare copies of the image patches using multiple color specifications. Color specification conversions can be selected based on a desired type of image augmentation (e.g., accentuating or boosting particular color channels, saturation levels, brightness levels, etc.). Color specification conversions can also be selected to improve compatibility between digital pathology image generation system 120 and the digital pathology image processing system 110. For example, a particular image scanning component can provide output in the HSL color specification and the models used in the digital pathology image processing system 110, as described herein, can be trained using RGB images. Converting the image patches to the compatible color specification can ensure the patches can still be analyzed. Additionally, the digital pathology image processing system can up-sample or down-sample images that are provided in a particular color depth (e.g., 8-bit, 1-bit, etc.) to be usable by the digital pathology image processing system. Furthermore, the digital pathology image processing system 110 can cause image patches to be converted according to the type of image that has been captured (e.g., fluorescent images may include greater detail on color intensity or a wider range of colors).

In some instances, the digital pathology image processing system 110 may detect one or more features from each of the plurality of image patches. The one or more features may comprise, for example, one or more of a clinical feature or a histologic feature, such as a cell type. Accordingly, generating the label for each of the plurality of image patches may be based on the one or more features. As an example, and not by way of limitation, clinical features may comprise one or more of patient age at diagnosis, patient sex, patient height, patient weight, patient clinical history, patient sample type, or patient smoking history. As another example, and not by way of limitation, histologic features may comprise, for example, growth patterns such as solid, cribriform, micropapillary, papillary, acinar, or lepidic.

As described herein, a patch-embedding module 112 can generate an embedding (e.g., a translation of a high dimensional vector representation of image features into a lower-dimensional space) for each image patch in a corresponding feature embedding space. The embedding can be represented by the digital pathology image processing system 110 as a feature vector for the image patch. In some instances, the patch-embedding module 112 may use a neural network (e.g., a convolutional neural network) to generate a feature vector that represents each patch of the image. In particular embodiments, the patch embedding neural network can be based on, e.g., the ResNet image network trained on a dataset based on natural (e.g., non-medical) images, such as the ImageNet dataset. By using a non-specialized image patch embedding network, the patch embedding module 112 can leverage known advances in efficiently processing images to generating embeddings. Furthermore, using a natural image dataset allows the embedding neural network to learn to discern differences between image patch segments on a holistic level.

In some instances, the image patch embedding network used by the patch embedding module 112 can be an embedding network customized to handle large numbers of image patches extracted from large format images, such as digital pathology whole slide images. Additionally, the patch embedding network used by the image patch embedding module 112 can be trained using a custom dataset. For example, the image patch embedding network can be trained using a variety of samples of whole slide images or even trained using samples relevant to the subject matter for which the embedding network will be generating embeddings (e.g., scans of particular tissue types). Training the image patch embedding network using specialized or customized sets of images can allow the image patch embedding network to identify finer (e.g., more subtle) differences between image patch features, which can result in more detailed and accurate distances between image patches in the feature embedding space at the potential cost of additional time to acquire the images and/or the computational and economic cost of training multiple image patch generating networks for use by the image patch embedding module 112. In some instances, the image patch embedding module 112 may select from a library of image patch embedding networks based on the type of images being processed by the digital pathology image processing system 110.

As described herein, image patch embeddings (e.g., lower-dimensional vector representations of image features) may be generated using a machine learning model, e.g., a deep learning neural network, based on visual features of the image patches. In some instances, the trained machine learning model may thus function as, e.g., an image feature extraction model. Image patch embeddings can be further generated from contextual information associated with the image patches or from the content shown in the image patch. For example, an image patch embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., based on dimensions expressed in pixels or converted from pixels to nanometers) or relative to other image patches from the same digital pathology image, from a class of digital pathology images (e.g., produced using similar techniques or by a single digital pathology image generation system or scanner), or from a related family of digital pathology images. Furthermore, image patches can be classified prior to using the image patch embedding module 112 to generate embeddings for the image patches such that the image patch embedding module 112 considers the classification when preparing the embeddings.

For consistency, in some instances the image patch embedding module 112 may produce embeddings of a predefined size (e.g., feature vectors of 512 elements, feature vectors of 2048 bytes, etc.). In some instances, the image patch embedding module 112 may produce embeddings of various and arbitrary sizes. The image patch embedding module 112 can adjust the sizes of the embeddings based on user direction, or sizes can be selected, for example, based on computation efficiency, accuracy, or other parameters. In particular instances, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

The digital pathology image processing system 110 can perform different inferences by applying one or more machine-learning models to the embeddings, i.e., inputting the embeddings to a machine-learning model. As an example, the digital pathology image processing system 110 can identify, based on a machine-learning model trained to identify tumor regions or tumor nest structures of cancer cells, a tumor region or tumor nest structure. In some instances, it may not be necessary to crop the image into image patches, generate embeddings for these image patches, and then perform inferences based on such embeddings. Instead, in some instances the digital pathology image processing system 110 with sufficient graphics processing unit (GPU) memory can directly apply the machine-learning model to the embedding of a whole slide image to make inferences. In some instances, the output of the machine-learning model may be resized into the shape of the input image.

A whole slide image access module 113 can manage requests to access whole slide images from other modules of the digital pathology image processing system 110 and the user device 130. For example, the whole slide image access module 113 may receive requests to identify a whole slide image based on a particular image patch, an identifier for the image patch, or an identifier for the whole slide image. The whole slide image access module 113 can perform tasks of confirming that the whole slide image is available to the requesting user or module, identifying the appropriate databases from which to retrieve the requested whole slide image, and retrieving any additional metadata that may be of interest to the requesting user or module. Additionally, the whole slide image access module 113 can handle efficient streaming of the appropriate data to the requesting device. As described herein, in some instances whole slide images may be provided to user devices in portions, based on the likelihood that a user will wish to see the entire while slide image or a portion of the whole slide image. In some instances, the whole slide image access module 113 may determine which regions of the whole slide image to provide and determine how to provide them. Furthermore, in some instances the whole slide image access module 113 may be empowered within the digital pathology image processing system 110 to ensure that no individual component locks up or otherwise misuses a database or whole slide image to the detriment of other components or users.

In some instances, an output generating module 114 of the digital pathology image processing system 110 can generate output corresponding to result image patch and result whole slide image datasets based on a user request. As described herein, the output can include a variety of visualizations, interactive graphics, and reports based upon the type of request and the type of data that is available. In some instances, the output will be provided to the user device 130 for display, but in certain instances the output may be accessed directly from the digital pathology image processing system 110. The output will be based on the existence of and access to the appropriate data, so the output generating module will be empowered to access necessarily metadata and anonymized patient information as needed. As with the other modules of the digital pathology image processing system 110, the output generating module 114 can be updated and improved in a modular fashion, so that new output features can be provided to users without requiring significant downtime.

The general techniques described herein can be integrated into a variety of tools and use cases. For example, as described, a user (e.g., pathologist or clinician) can access a user device 130 that is in communication with the digital pathology image processing system 110 and provide a query image for analysis. The digital pathology image processing system 110, or the connection to the digital pathology image processing system 110 can be provided as a standalone software tool or package that searches for corresponding matches, identifies similar features, and generates appropriate output for the user upon request. As a standalone tool or plug-in that can be purchased or licensed on a streamlined basis, the tool can be used to augment the capabilities of a research or clinical lab. Additionally, the tool can be integrated into the services made available to the customer of digital pathology image generation services laboratory. For example, the tool can be provided as part of a unified workflow, where a user who conducts research or requests a whole slide image to be created for a submitted sample automatically receives a report of noteworthy features within the image and/or similar whole slide images that have been previously indexed. Therefore, in addition to improving whole slide image analysis, the techniques can be integrated into existing systems to provide additional features not previously considered or possible.

Moreover, the digital pathology image processing system 110 can be trained and customized for use in particular settings. For example, the digital pathology image processing system 110 can be specifically trained for use in providing insights relating to specific types of tissue (e.g., lung, heart, blood, liver, etc.). As another example, the digital pathology image processing system 110 can be trained to assist with safety assessment, for example in determining levels or degrees of toxicity associated with drugs or other potential therapeutic treatments. Once trained for use in a specific subject matter or use case, the digital pathology image processing system 110 is not necessarily limited to that use case. Training may be performed in a particular context, e.g., toxicity assessment, due to a relatively larger set of at least partially labeled or annotated images.

The methods and systems disclosed herein may enable users to easily request subject predictions based on digital pathology images provided by the user. In some instances, the digital pathology image processing system 110 may transmit, from a client computing system to a remote computing system, a request communication to process a digital pathology image that depicts cancer cells in a particular section of a biological sample from a subject. In response to receiving the request communication from the client computing system, the remote computing system may perform operations comprising the following steps. The remote computing system may first access the digital pathology image. The remote computing system may then segment the digital pathology image into a plurality of image patches. The remote computing system may then generate, for each of the plurality of image patches, a label indicating whether the image patch depicts, e.g., a tumor region or a tumor nest structure. The remote computing system may then determine, based on the labels generated for each image patch, that the digital pathology image comprises a depiction of an occurrence of gene fusion with respect to the cancer cells. The remote computing system may then generate, based on the occurrence of gene fusion with response to the cancer cells, a subject prediction for the subject. In some instances, the subject prediction may comprise a prediction of applicability of one or more treatment regimens for the subject. The remote computing system may further provide the subject prediction to the client computing system via a response communication. In some instances, the client computing system may output the subject prediction in response to receiving the response communication.

### Machine Learning Model Training Workflow-Exemplary Scenario 1

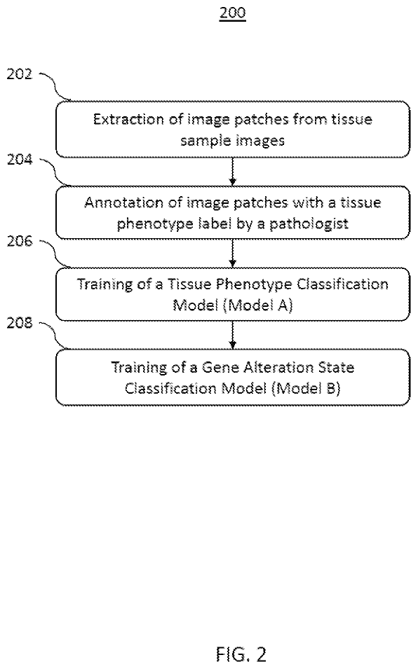

FIG. 2 provides a first non-limiting example of the steps taken to train a tissue phenotype classification model and a gene alteration state classification model in a process 200, according to one implementation of the disclosed methods for image-based prediction and/or determination of altered gene states in a tissue sample.

As illustrated in FIG. 2, image patches are extracted from at least a subset of images, 202, from a cohort of tissue sample images of interest (e.g., whole slide pathology images of tissue samples that individually exhibit the characteristic of interest, such as a specific gene alteration state as determined using genotyping or next-generation sequencing (NGS) methods. Image patches may be extracted from whole slide images using any of a variety of image processing techniques known to those of skill in the art. For example, in some instances a down-sampled image (e.g., a thumbnail) of the full-sized megapixel or gigapixel image is utilized to make masking and image patch coordinate selection more tractable than when processing the full sized image. In some instances, the down-sampled image used may be the smallest image provided in the image pyramid for the specific image format used (e.g., .svs image files, .tiff image files, etc.). The down-sampled image may than be converted from color to grayscale using standard conversion algorithms included in image processing libraries. A thresholding technique such as Otsu's method is applied to the grayscale image to generate a binary mask. The binary mask can be cleaned using techniques such as binary erosion and dilation (a process that removes small specks and islands from the binary image), as well as other small object removal techniques. Once the binary mask has been cleaned, it is used as a sampling map for image patch extraction. In image patch extraction, the desired size of the full-resolution image patches are typically predetermined. A scaling factor is then calculated for the down-sampled image being processed and the full size image, so that the scaled down-sampled image patch size can be used to loop (e.g., raster) through the down-sampled mask. At each point during the looping process, the local down-sampled image patch is assessed to ensure that there is enough tissue present (compared to a predetermined percentage threshold), and if so, the down-sampled image coordinates of that image patch are stored. Once the down-sampled mask has been processed for patch extraction, the associated down-sampled patch coordinates are converted to the full size image coordinates which are then used to ‘extract’image patches from the full resolution image in the image pyramid. Often, ‘extracting’ patches is equivalent to selectively reading out only a small (image patch sized) portion of the full image.

The image patches for a subset of the image cohort are then annotated by trained pathologists, 204, to indicate tissue phenotype classes of interest. In some instances, annotation of whole slide images by a pathologist may be performed prior to performing image patch extraction. The annotated image patch data comprising the pre-determined labels (e.g., tissue phenotype classes of interest) provided by the trained pathologists are then used as a labeled image patch training data set to train a tissue phenotype classification model (Model A).

As indicated in step 206, training of the tissue phenotype classification model (Model A) (e.g., a convolutional neural network) to classify image patches as belonging to the one or more tissue phenotype classes is accomplished using the labeled image patch training data and any of a variety of machine learning/optimization approaches (e.g., gradient descent method, a Newton method, a conjugate gradient method, a quasi-Newton method, or a Levenberg-Marquardt method). The trained tissue phenotype classification model (Model A) is then used to classify tissue image patches extracted from all remaining slides in the cohort of tissue sample images.

In some instances, iteration through labeled image patch data for each tissue phenotype class of interest (or combinations of tissue phenotype classes), may be performed to assess their use (along with paired slide-level labels for the corresponding gene alteration state as determined by genotyping or next generation sequencing) in training a downstream machine learning model (e.g., a convolutional neural network) to predict the correct gene alteration state. Iteration through labeled image patch data for each phenotype class thus allows one to identify the set of labeled image patch data that is the best predictor of the gene alteration state of interest.

Finally, as indicated in step 208 of FIG. 2, a subset of the labeled image patch data belonging to the “signal-containing” tissue phenotype classes that provide the best indicator(s) for a given gene alteration state are used to further train, tune, and deploy a gene alteration state classification model (Model B) that is configured to determine and output a slide-level gene alteration state for a tissue sample. In some instances, slide level calls of gene alteration state may comprise the use of an image patch prediction aggregation technique, such as averaging over individual image patch predictions of gene alteration state to make the slide-level determination, or by using a “majority vote” approach as will be discussed in more detail below.

### Machine Learning Model Training Workflow-Exemplary Scenario 2

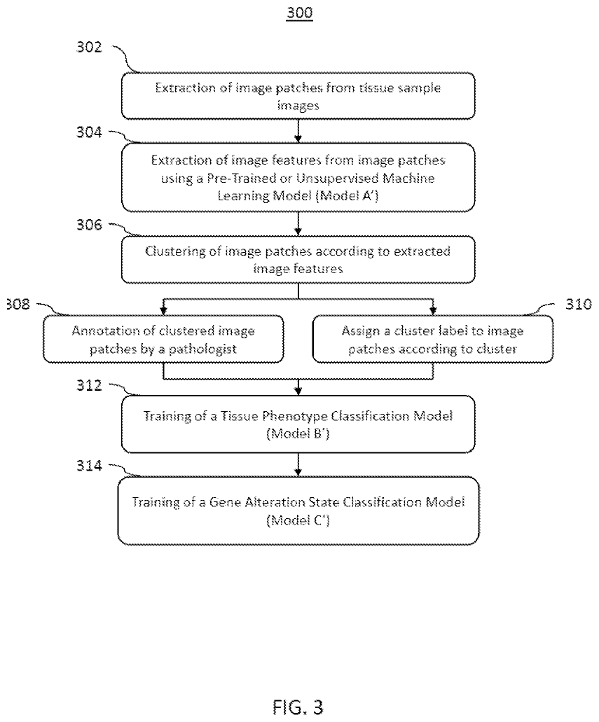

FIG. 3 provides a second non-limiting example of the steps taken to train a tissue phenotype classification model and a gene alteration state classification model according to another implementation of the disclosed methods.

As illustrated in FIG. 3, image patches are extracted from at least a subset of images, at step 302, from a cohort of tissue sample images of interest (e.g., whole slide pathology images of tissue samples that individually exhibit the characteristic of interest such as a specific gene alteration state as determined using genotyping or next-generation sequencing (NGS) methods.

Image feature extraction is performed, 304, on the tissue image patches (i.e., feature-extraction image patches) extracted from the subset of images in order to cluster them by visual similarity. Extraction of image features by an upstream machine learning model (Model A′) is performed using either a pre-trained machine learning model (e.g., a convolutional neural network model) as a fixed-feature extractor, or an unsupervised machine learning model (e.g., an autoencoder) which is trained on the visual distribution of the cohort of interest.

Clustering of the feature-extraction image patches based on the extracted image features is then performed, 306. In some instances, feature extraction and clustering may be performed using different machine learning models and/or statistical approaches. In some instances these steps may be performed using a machine learning model or software module that comprises both the feature extraction method and the clustering method.

In some instances, as illustrated in step 308, pathologists may characterize and annotate (label) each image patch cluster (and the corresponding feature-extraction image patches within the cluster) with corresponding tissue phenotype classes and/or other relevant patient and/or tissue sample details.

In some instances, the feature-extraction image patches in each cluster may be assigned a cluster label, 310, that may or may not correlate with visually identifiable tissue phenotype classes.

As illustrated in step 312, labeled image patch data selected from image feature clusters of interest is then used to train a machine learning model (e.g., a convolutional neural network), as a tissue phenotype classification model (Model B′) configured to classify non-labeled image patches into the tissue phenotype classes of interest (thus generating image patch data labeled with tissue phenotype class and/or image feature cluster labels). The trained tissue phenotype classification model (model B′) is then used to classify all image patches extracted from the remaining slides from the cohort.

In some instances, iteration through labeled image patch data for each tissue phenotype class or image feature cluster of interest (or combinations of tissue phenotype classes or image feature clusters) may be performed and used to assess of their use (along with paired slide-level labels for the corresponding gene alteration state as determined by genotyping or next generation sequencing) in training a downstream machine learning model, e.g., a convolutional neural network (model C), to predict the correct gene alteration state Iteration through labeled image patch data for each phenotype class thus allows one to identify the set of labeled image patch data that is the best predictor of the gene alteration state of interest.

Finally, as illustrated in step 314 of FIG. 3, the subset of image patches belonging to the “signal-containing” classes (determined by iterative experimentation) are used to further train, tune, and deploy a gene alteration state classification model (Model C′) that is configured to provide an image-based determination of a slide-level gene alteration state for the tissue sample where, in some instance, the slide-level determination of gene alteration state for the tissue sample that is output by the model is based on, e.g., aggregating individual image patch predictions by averaging, or using a “majority vote” approach as will be discussed in more detail below. In some instances, a slide-level determination of gene alteration state may be achieved by extracting image features from the subset of labeled image patches belonging to the “signal-containing” classes, aggregating those image features, and training the gene alteration state prediction model using the aggregated image features and associated slide-level gene alteration state label.

### Deployment and Methods of Use

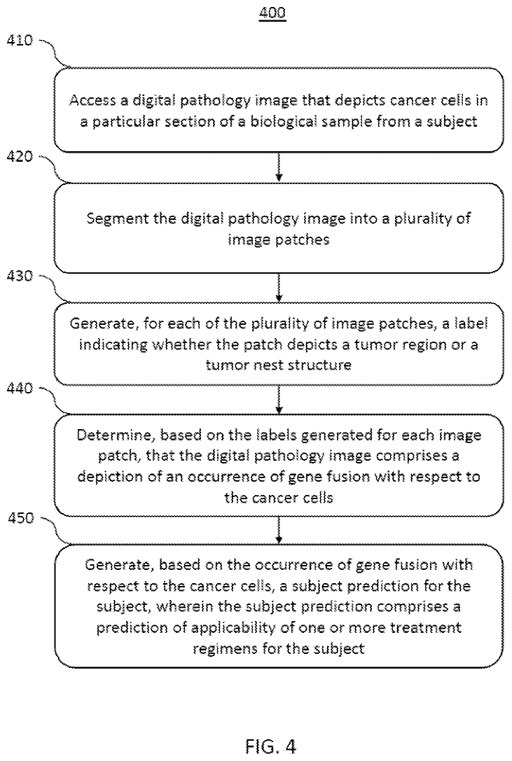

FIG. 4 illustrates an example method 400 for detecting gene alterations, e.g., gene fusions. The method may include step 410, where the digital pathology image processing system 110 depicted in FIG. 1 may access a digital pathology image that depicts cancer cells in a particular section of a biological sample from a subject. As an example, and not by way of limitation, the digital pathology image may be a scanned, stained (e.g., hematoxylin and eosin stained) whole slide image of the biological sample that includes tumorous cells (e.g., lung adenocarcinoma).

At step 420 in FIG. 4, the digital pathology image processing system 110 may segment the digital pathology image into a plurality of image patches. In particular instances, the patch-generating module 111 depicted in FIG. 1 may be used to generate the image patches. The patches may be non-overlapping or overlapping. Features such as whether or not image patches overlap, in addition to the size of each image patch and the step-wise displacement of the window used to create image patches can increase or decrease the data set for analysis, with more image patches increasing the potential resolution of the eventual output and visualization. In particular instances, each image patch may be of a predefined size and/or an offset between image patches may be predefined. Furthermore, the patch-generating module 111 may create multiple sets of image patches of varying size, overlap, step size, etc., for each image. The patch-generating module 111 may generate image patches for each digital pathology image in one or more color channels or for one or more color combinations. The image patches may be generated based on segmenting the color channels and/or generating a brightness map or greyscale equivalent of each image patch. Additionally, the digital pathology image processing system 110 can up-sample or down-sample images that are provided in a particular color depth to be usable by the digital pathology image processing system 110. Furthermore, the digital pathology image processing system 110 can cause image patches to be converted according to the type of image that has been captured.

At step 430 in FIG. 4, the digital pathology image processing system 110 may generate, for each of the plurality of image patches, a label indicating whether the image patch depicts a tumor region or a tumor nest structure. As an example, and not by way of limitation, the digital pathology image processing system 110 may detect one or more features from each of the plurality of image patches. The one or more features may comprise, e.g., one or more of a histologic feature, such as a cell type or cell grouping, a clinical feature, or a genomic feature. Accordingly, generating the label for each of the plurality of image patches may be based on the one or more features. In particular instances, generating the label for each of the plurality of image patches may be based on one or more of image patch-based classification or multi-instance learning (MIL) classification. As noted elsewhere herein, generating the label for each of the plurality of image patches may be based on the use of one or more trained machine-learning models. In particular instances, the digital pathology image processing system 110 may train the one or more machine-learning models based on a plurality of training data comprising one or more labeled depictions of a sample comprising, e.g., a tumor region or tumor nest structure, and one or more labeled depictions of a sample that does not include a tumor region or tumor nest structure. In some instances, generating the label for each of the plurality of image patches may be based on tissue morphology, e.g., tumor morphology. The tumor morphology may be based on, for example, an analysis of one or more of the following histologic features: the presence or number of signet ring cells, the presence or number of hepatoid cells, extracellular mucin, a tumor growth pattern, or tumor heterogeneity.

At step 440 in FIG. 4, the digital pathology image processing system 110 may determine, based on the labels generated for each image patch, that the digital pathology image comprises a depiction of an occurrence of gene fusion with respect to the cancer cells in the image. In particular instances, the digital pathology image processing system 110 may use any of a variety of different approaches for effectively determining that gene fusions are present. One approach, for example, may comprise combining a target gene fusion (e.g., an NTRK fusion) with other gene fusions, such as ROS1, ALK, and RET fusions, into a single actionable gene fusion cluster. The cluster may be then treated as a single category of gene fusion to facilitate detection. In this approach, rather than trying to identify each gene fusion individually, the digital pathology image processing system 110 instead treats them as a single group and thus is no longer required to detect gene fusions that occur individually with a frequency of less than half a percent. As an example, and not by way of limitation, the combined frequency of occurrence for these gene fusions may be about 15 percent.

Another approach may comprise using the molecular landscape and molecular features of these tumors. In particular instances, signals for fusions may arise primarily in tumor nests/cells and may be strong and diffuse across the tumor area. Therefore, in addition to identifying fusions directly from the slide, the digital pathology image processing system 110 may identify gene fusions based on the mutually-exclusive distribution of molecular features across tumors. For example, the morphology of lung adenocarcinoma may be mapped onto the molecular landscape, which may comprise 17% EGFR-sensitizing, 7% ALK, 4% EGFR other, 3% having >1 mutation, 2% HER2, 2% ROS1, 2% BRAF, 2% RET, 1% NTRK1, 1% PIK3CA, 1% MEK1, 31% unknown oncogenic driver, and 25% KRAS alterations. Among the most common driver mutations of lung adenocarcinoma, only three percent may have greater than one mutation, which means that 97% of lung cancer patients carry a single mutation. It is therefore significantly more common for driver mutations to display mutual exclusivity, and this feature may be used in a variety of contexts to inform clinical decision making in the treatment of cancer patients. In some instances of the disclosed methods and systems, the digital pathology image processing system 110 may access a digital pathology image that depicts cancer cells in a particular section of a biological sample from a subject. The digital pathology image processing system 110 may then determine that the digital pathology image comprises a depiction of one or more mutations that are mutually exclusive with an occurrence of gene fusion, and thus determine an absence of gene fusion with respect to the cancer cells. In some instances, the digital pathology image processing system 110 may further generate, based on the absence of a gene fusion with respect to the cancer, a subject prediction for the subject. The subject prediction may comprise, for example, a prediction of the applicability of one or more treatment regimens for the subject. Because of this mutual exclusivity, aside from positively identifying the gene fusion, the digital pathology model may identify more common mutations such as KRAS and EGFR and in doing so, rule out the presence of a gene fusion.

In some instances, predicting or determining that an actionable gene fusion is present may be based, at least in part, on identifying and ruling out a smoking-related mutational signature (see, e.g., Alexandrov, et al. (2016), “Mutational Signatures Associated With Tobacco Smoking in Human Cancer”, Science 354(6312): 618-622).

In some instances, predicting or determining that an actionable gene fusion is present may be based on the detection of histologic features associated with ALK, ROS1, and RET, including solid and cribriform growth patterns, and/or extracellular mucin. As another example, and not by way of limitation, predicting or determining that an actionable gene fusion is present may be based on the detection of cell types associated with ALK, ROS1, and RET. These cell types may comprise one or more of signet ring cells, goblet cells, or hepatoid cells. Different features may have different levels of importance to different tumor types. Automatic detection and quantification of each of these visual features may allow for prediction of, for example, ALK, ROS1, RET and NTRK alterations in, for example, lung adenocarcinoma.

In some instances, another feature of gene fusions and tumors that may be used by the digital pathology image processing system 110 (or the digital pathology machine learning model residing therein) for determining the presence of gene alterations, e.g., gene fusions, may be tumor mutational burden (TMB). In some instances, for example, kinase or oncogene fusions may be associated with low tumor mutational burden. A tumor's main oncogenic driver may be a single gene fusion. Therefore, one may expect that the morphologic signal derived from a single oncogenic driver would be present across the majority of tumor cells/areas in a tissue specimen on a slide. End-to-end gene fusion status prediction may also show strong uniform signal across the whole slide.

In some instances, low tumor mutational burden may suggest decreased tumor morphologic heterogeneity. Patients may be characterized as having a driver mutation, a mutation in a driver gene, and/or a driver fusion (e.g., a gene fusion involving a driver gene). In some instances, the tumor mutational burden in cancers may be driven by a driver mutation. In some instances, the tumor mutational burden of cancers may be also driven by a gene fusion. In some instances, cancer driven by a gene fusion may have a significantly lower tumor mutational burden. Therefore, a low tumor mutational burden may be associated with a low tumor heterogeneity.

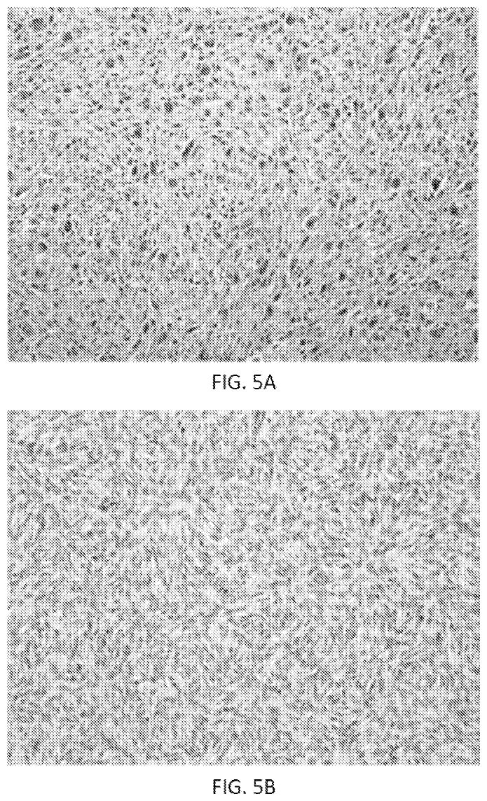

In some instances, predicting or determining that an actionable gene fusion is present may be based, at least in part, on the detection of tumor heterogeneity. FIGS. 5A and 5B provide examples of tumor heterogeneity as visualized in digital pathology images. Tumor heterogeneity is a global descriptor applicable to all tumor cells regardless of histologic type. Tumor heterogeneity may allow one to identify gene fusions across many different histologic types, i.e., lack of tumor heterogeneity may be associated with gene fusions across many different histologic types. FIG. 5A and FIG. 5B may correspond to two different kinds of sarcomas. FIG. 5A provides an example tumor heterogeneity in uterine leiomyosarcoma. The tumor cells look very different from each other. In other words, they are very heterogeneous. FIG. 5B provides an example tumor heterogeneity in dermatofibrosarcoma protuberans. The tumor cells as a population are more similar in terms of their shape, chromatic intensity, angularity, and size. In sarcomas, high heterogeneity is often associated with aneuploidy which is an aberration of chromosome number, whereas monotony (e.g., low tumor heterogeneity) may be associated with translocations (i.e., another name for gene fusions). Lack of tumor heterogeneity may thus be the signature for gene fusions across tumor types. However, in some instances it may be difficult for human eyes to observe the lack of tumor heterogeneity. In such instances, the digital pathology methods described herein may be particularly helpful as machine learning approaches are more suitable for identifying subtle differences which may require exhaustive analysis.

Tumor heterogeneity can be a sign of aggressive disease. Patients with gene fusions often present at a high stage of disease. The morphologic features of tumor heterogeneity which are seen in these patients may be features of aggressive tumor behavior, except that in some cases their cells may have low heterogeneity (i.e., their cells may look clonal). On a cellular level, the cells may appear clonal, and yet on the group level (e.g., the population level), their phenotype may be aggressive, which may be specific to the presence of one or more gene fusions. There are at least a couple of hypotheses regarding the correlation of tumor heterogeneity with gene fusion. One hypothesis may be that the visual signal that is indicative for gene fusions may reside primarily in tumor nests/cells. Another hypothesis may be that the visual signal that is indicative of gene fusion may be strong and diffuse across all parts of the tumor area. Yet another hypothesis may be that low tumor mutational burden may suggest decreased tumor morphologic heterogeneity.

To detect a lack of tumor heterogeneity, in some instances the digital pathology image processing system 110 may analyze each tumor nucleus identified in the whole slide image using any of several different approaches. For example, in one approach automatic tumor nuclei detection and parameterization may be performed, in which a trained machine-learning model may be used to identify each tumor nucleus, measure a set of specified parameters or features for each nucleus, as discussed below, and then compare the population-level distribution of the specified parameters or features. In another example, the approach may comprise performing tumor image segmentation, which may be an image patch-based assessment. In some instances, determination of tumor heterogeneity may be performed on a per slide prediction basis (which may include, for example, calculating percentage(s) of image patches predicted to be heterogeneous, or averages of each slide's prediction scores).

In some instances, tumor heterogeneity may be driven by the type of gene involved in a gene fusion. For tumor suppressor genes, oncogenesis may be mediated by loss of function. Such mutations can release the cell from normal cell cycle control, which in turn may indirectly promote growth. Over time, this process allows for the accumulation of cancer-promoting mutations with each new generation of daughter cells. In contrast, for oncogenes, oncogenesis may be mediated by gain of function. Over-activation of growth factors, for example, may directly promote growth, thereby resulting in unrestricted growth. This process is predicted to result in an immediate growth advantage that does not require additional mutations. Based on this rationale, low tumor heterogeneity may be expected in tumors comprising a fusion involving an oncogene, such as ALK, ROS1, RET and NTRK.

One approach to assessing tumor heterogeneity may include assessment of the morphology of cellular-level structures in tumor cells, such as nuclei. The morphology of nuclei may be represented by a plurality of image features, which may be organized into categories of image features, such as, by way of example and not limitation, chromatin features, geometric coordinates, basic morphology features, two-dimensional shape features, first-order statistics, “gray-level” (e.g., where “gray” represents a spatial distribution of pixel intensity levels) co-occurrence matrix features, gray-level dependence matrix features, gray-level run length matrix features, gray-level size zone matrix features, neighboring gray-tone difference matrix features, advanced nucleus morphology features, and boundary and curvature features.

Each type may comprise one or more image features. Example image features may include, but are not limited to:
chromatin features, such as:
   heterogeneity of the nucleus (hetero),
   the size distribution of granules (clump),
   the fraction of large granules with respect to total nuclear area (condense),
   the distribution around the nuclear membrane or margin (margination);
geometric coordinates, such as:
   the region coordinates (region_coords),
   the x coordinates (x),
   the y coordinates (y);
basic morphology features, such as:
   area of the nucleus (area),
   convex area of the nucleus (convex_area),
   eccentricity of the nucleus (eccentricity),
   diameter of the nucleus (equivalent_diameter),
   the ratio of pixels in the nucleus to pixels in a bounding box, either in a total field or view or a selected area of a field of view (extent),
   perimeter of the nucleus (perimeter),
the ratio of pixels in the nucleus to pixels of a convex hull (solidity),
   the elongation of the nucleus as measured by eigenvalues of the inertia tensor (inertia_tensor_eigvals1, inertia_tensor_eigvals2),
   the length of the major axis of the nucleus (major_axis_length),
   the length of the minor axis of the nucleus (minor_axis_length),
   Hu moments (a certain particular weighted average, a.k. a. “moment,” of the image pixels' intensities, i.e., translation, rotation, and scale-invariant moments) (moments_hu0, moments_hu1, moments_hu2, moments_hu3, moments_hu4, moments_hu5, moments_hu6),
   weighted Hu moments (weighted_moments_hu0, weighted_moments_hul, weighted_moments_hu2, weighted_moments_hu3, weighted_moments_hu4, weighted_moments_hu5, weighted_moments_hu6);
two-dimensional shape features, such as:
   two-dimensional shape elongation (original_shape2D_Elongation),
   two-dimensional shape maximum diameter (original_shape2D_MaximumDiameter),
   two-dimensional shape mesh surface (original_shape2D_MeshSurface),
   two-dimensional shape perimeter-to-surface ratio (original_shape2D_PerimeterSurfaceRatio),
   two-dimensional shape pixel surface (original_shape2D_PixelSurface),
   two-dimensional shape sphericity (original_shape2D_Sphericity),
   two-dimensional shape spherical disproportion (original_shape2D_SphericalDisproportion);
first-order statistics, such as:
   first-order 10^{th} percentile (original_firstorder_10Percentile),
   first-order 90^{th} percentile (original_firstorder_90Percentile),
   first-order energy (original_firstorder_Energy),
   first-order entropy, which specifies the uncertainty or randomness in the image values (original_firstorder_Entropy),
   first-order interquartile range, which measures the variability based on quartile splitting (original_firstorder_InterquartileRange),
   first-order kurtosis, which measures the “peakedness” of the distribution of values (original_firstorder_Kurtosis),
   first-order maximum (original_firstorder_Maximum),
   first-order mean absolute deviation (original_firstorder_MeanAbsoluteDeviation),
   first-order mean (original_firstorder_Mean),
   first-order median (original_firstorder_Median),
   first-order minimum (original_firstorder_Minimum),
   first-order range (original_firstorder_Range),
   first-order robust man absolute deviation, which is the mean distance of all intensity values from the mean value (original_firstorder_RobustMeanAbsoluteDeviation),
   first-order root mean squared, which is the mean of all squared intensity values (original_firstorder_RootMeanSquared),
   first-order skewness, which measures the asymmetry of the distribution of values about the mean (original_firstorder_Skewness),
   first-order total energy (original_firstorder_TotalEnergy),
   first-order uniformity (original_firstorder_Uniformity),
   first-order variance (original_firstorder_Variance);
gray-level co-occurrence matrix (GLCM) (describes the second-order joint probability function of an image region constrained by the mask) features, such as GLCM autocorrelation (original_glcm_Autocorrelation),
   GLCM cluster prominence (original_glcm_ClusterProminence),
   GLCM cluster shade (original_glcm_ClusterShade),
   GLCM cluster tendency (original_glcm_ClusterTendency),
   GLCM contrast (original_glem_Contrast),
   GLCM correlation (original_glem_Correlation),
   GLCM difference average (original_glem_DifferenceAverage),
   GLCM difference entropy (original_glcm_DifferenceEntropy)
   GLCM difference variance (original_glcm_Difference Variance),
   GLCM inverse difference (original_glcm_Id),
   GLCM inverse difference moment (original_glcm_Idm),
   GLCM inverse difference moment normalized (original_glcm_Idmn),
   GLCM inverse difference normalized (original_glcm_Idn),
   GLCM informational measure of correlation (original_glcm_Imc1, original_glcm_Imc2),
   GLCM inverse variance (original_glcm_Inverse Variance),
   GLCM joint average (original_glcm_JointAverage),
   GLCM joint energy (original_glcm_JointEnergy),
   GLCM joint entropy (original_glcm_JointEntropy),
   GLCM maximal correlation coefficient (original_glcm_MCC),
   GLCM maximum probability (original_glem_MaximumProbability),
   GLCM sum average (original_glcm_SumAverage),
   GLCM sum entropy (original_glcm_SumEntropy),
   GLCM sum squares (original_glcm_SumSquares);
gray-level dependence matrix (quantifies gray level dependencies in an image, wherein a gray level dependency is defined as the number of connected pixels within a specified distance that are dependent on the center pixel) features, such as:
   GLDM gray level dependence entropy (original_gldm_DependenceEntropy),
   GLDM dependence nonuniformity (original_gldm_DependenceNonUniformity),
   GLDM dependence nonuniformity normalized (original_gldm_DependenceNonUniformityNormalized),
   GLDM dependence variance (original_gldm_DependenceVariance),
   GLDM gray-level nonuniformity (original_gldm_GrayLevelNonUniformity),
   GLDM gray-level variance (original_gldm_GrayLevelVariance),
   GLDM high gray-level emphasis (original_gldm_HighGrayLevelEmphasis),
   GLDM large dependence emphasis (original_gldm_LargeDependenceEmphasis),
   GLDM large dependence high gray-level emphasis (original_gldm_LargeDependenceHighGrayLevelEmphasis),
   GLDM large dependence low gray-level emphasis (original_gldm_LargeDependenceLowGrayLevelEmphasis),
   GLDM low gray-level emphasis (original_gldm_LowGrayLevelEmphasis),
   GLDM small dependence emphasis (original_gldm_SmallDependenceEmphasis),
   GLDM small dependence high gray-level emphasis (original_gldm_SmallDependenceHighGrayLevelEmphasis),
   GLDM small dependence low gray-level emphasis (original_gldm_SmallDependenceLowGrayLevelEmphasis);
gray-level run length matrix (GLRLM) (quantifies gray level runs, which are defined as the length in number of pixels, of consecutive pixels that have the same gray level value) features, such as:
   GLRLM gray-level nonuniformity (original_glrlm_GrayLevelNonUniformity),
   GLRLM gray-level nonuniformity normalized (original_glrlm_GrayLevelNonUniformityNormalized),
   GLRLM gray-level variance (original_girlm_GrayLevelVariance),
   GLRLM high gray-level run emphasis (original_glrlm_HighGrayLevelRunEmphasis),
   GLRLM long-run emphasis (LRE) (original_glrlm_LongRunEmphasis),
   GLRLM long-run high gray-level emphasis (original_glrlm_LongRunHighGrayLevelEmphasis),
   GLRLM long-run low gray-level emphasis (original_glrm_LongRunLowGrayLevelEmphasis),
   GLRLM low gray-level run emphasis (original_glrlm_LowGrayLevelRunEmphasis),
   GLRM run entropy (original_glrm_RunEntropy),
   GLRM run length nonuniformity), (original_glrm_RunLengthNonuniformity),
   GLRLM run length nonuniformity normalized (original_glrlm_RunLengthNonUniformityNormalized),
   GLRLM run percentage (original_glrlm_RunPercentage),
   GLRLM run variance (original_glrlm_RunVariance),
   GLRLM short run emphasis (original_girlm_ShortRunEmphasis),
   GLRLM short run high gray-level emphasis (original_glrlm_ShortRunHighGrayLevelEmphasis),
   GLRLM short run low gray-level emphasis (original_glrlm_ShortRunLowGrayLevelEmphasis);
gray-level size zone (GLSZM) (describes gray-level zones in an image region) matrix features, such as:
   GLSZM gray-level nonuniformity (original_glszm_GrayLevelNonUniformity),
   GLSZM gray-level nonuniformity normalized (original_glszm_GrayLevelNonUniformityNormalized),
   GLSZM gray-level variance (original_glszm_GrayLevelVariance),
   GLSZM high gray-level zone emphasis (original_glszm_HighGrayLevelZoneEmphasis),
   GLSZM large area emphasis (original_glszm_LargeAreaEmphasis),
   GLSZM large area high gray-level emphasis (original_glszm_LargeAreaHighGrayLevelEmphasis),
   GLSZM large area low gray-level emphasis (original_glszm_LargeAreaLowGrayLevelEmphasis),
   GLSZM low gray-level zone emphasis (original_glszm_LowGrayLevelZoneEmphasis),
   GLSZM size zone nonuniformity (original_glszm_SizeZoneNonUniformity),
   GLSZM size zone nonuniformity normalized (original_glszm_SizeZoneNonUniformityNormalized),
   GLSZM small area emphasis (original_glszm_SmallAreaEmphasis),
   GLSZM small area emphasis high gray-level emphasis (original_glszm_SmallAreaHighGrayLevelEmphasis),
   GLSZM small area low gray-level emphasis (original_glszm_SmallAreaLowGrayLevelEmphasis),
   GLSZM zone entropy (original_glszm_ZoneEntropy),
   GLSZM zone percentage (original_glszm_ZonePercentage),
   GLSZM zone variance(original_glszm_ZoneVariance);
neighboring gray-tone difference matrix (NGTDM) (describes the difference between a gray value and the average gray value of neighbors within a certain distance) features, such as:
   NGTDM busyness (original_ngtdm_Busyness),
   NGTDM coarseness (original_ngtdm_Coarseness),
   NGTDM complexity (original_ngtdm_Complexity),
   NGTDM contrast (original_ngtdm_Contrast),
   NGTDM strength (original_ngtdm_Strength);
advanced nucleus morphology features, such as:
   radius of an ellipse-shaped nucleus (ellipse_R_index),
   major axis of an ellipse-shaped nucleus (ellipse_MA_index),
   convexity perimeter of a nucleus, which measures the perimeter of curvature (convexity_perimeter),
   circularity of a nucleus, which measures the roundness of a nucleus (circularity),
   normalized number of connected components that remain when a shape is subtracted from a convex hull (Ncce_index);
boundary (where a boundary signature of a nucleus is the distance profile from all boundary coordinates to the centroid points of the nucleus) features, such as:
   mean (mean(R)(≡<R>)),
   median (median(R)),
   mode (mode(R)),
   maximum (max_v: maxR(R)),
   minimum (min_v: min(R)),
   25^{th} percentile of a boundary signature (percentile_25: 25% percentile (R)),
   75^{th} percentile of a boundary signature (percentile_75: 75% percentile (R)),
   below 25^{th} percentile of mean boundary signature (mean_below_percentile_25: mean(R(R<percentile_25))),
   above 75^{th} percentile of mean boundary signature (mean_above_percentile_75: mean(R(R>percentile_75))),
   sum distance of a boundary signature (sum_dist: sum(R)),
   harmonic mean of a boundary signature (harmonic_mean: harmonic mean(R)),
   3% trimmed mean boundary signature (trimmed_mean_3_percent: 3% trimmed mean(R)),
   5% trimmed mean boundary signature (trimmed_mean_5_percent: 5% trimmed mean(R)),
   15% trimmed mean boundary signature (trimmed_mean_15_percent: 15% trimmed mean(R)),
   25% trimmed mean boundary signature (trimmed_mean_25_percent: 25% trimmed mean(R)),
   standard deviation (std_dev: standard deviation(R) (≡sR)),
   standard deviation by mean (std_dev_by_mean: sR/|<R>|),
   standard deviation by median (std_dev_by_median: sR/|median(R)|),
   standard deviation by mode (std_dev_by_mode: sR/|mode(R)|),
   skewness (skewness(R)),
   kurtosis (kurtosis(R)),
   mean distance profile minus mean of a boundary signature (mean_dist_profile_minus_mean: mean(|R−<R>|)),
   range (range_v: range(X)),
   interquartile range (interquantile_range: interquartile range (X)),
   sum distance profile squared (sum_dist_profile_square: sum(R2)),
   sum distance profile cubed (sum_dist_profile_cube: sum(R3)),
   mean distance profile squared (mean_dist_profile_square: mean(R2)),
   mean distance profile cubed (mean_dist_profile_cube: mean(R3)),
   mean distance profile raised to four (mean_dist_profile_raise_to_four: mean(R4)),
   mean distance profile raised to five (mean_dist_profile_raise_to_five: mean(R5)),
   sum distance profile minus mean power of 2 (sum_dist_profile_minus_mean_pow2: sum(|R−<R>|2)),
   sum distance profile minus mean power of 3 (sum_dist_profile_minus_mean_pow3: sum(|R−<R>|3)),
   mean distance profile minus mean power of 2 (mean_dist_profile_minus_mean_pow2: mean(|R−<R>|2)),
   mean distance profile minus mean power of 3 (mean_dist_profile_minus_mean_pow3: mean(|R−<R>|3)),
   mean distance profile minus mean power of 4 (mean_dist_profile_minus_mean_pow4: mean(|R−<R>|4)),
   mean distance profile minus mean power of 5 (mean_dist_profile_minus_mean_pow5: mean(|R−<R>|5)),
   number of peaks (number_of_peaks),
   gini coefficient (gini coefficient);
curvature features, such as:
   mean curvature (c_mean: mean(k) (≡<k>)),
   median curvature (c_median: median(k)),
   mode curvature (c_mode: mode(k)),
   maximum curvature (c_max_v: max(k)),
   minimum curvature (c_min_v: min(k)),
   25^{th} percentile curvature (c_percentile_25: 25% percentile (k)),
   75^{th} percentile curvature (c_percentile_75: 75% percentile (k)),
   below 25^{th} percentile of the mean curvature (c_mean_below_percentile_25: mean(k(k<c_percentile_25))),
   above 75^{th} percentile of the mean curvature (c_mean_above_percentile_75: mean(k(k>c_percentile_75))),
   sum dance (c_sum_dist: sum(k)),
   harmonic mean (c_harmonic_mean: harmonic mean(k)),
   3% trimmed mean curvature (c_trimmed_mean_3_percent: 3% trimmed mean(k)),
   3% trimmed mean curvature (c_trimmed_mean_5_percent: 5% trimmed mean(k)),
   15% trimmed mean curvature (c_trimmed_mean_15_percent: 15% trimmed mean(k)),
   25% trimmed mean curvature (c_trimmed_mean_25_percent: 25% trimmed mean(k)),
   standard deviation (c_std_dev: standard deviation(k) (≡sk)),
   standard deviation by mean (c_std_dev_by_mean: sk/|<k>|),
   standard deviation by median (c_std_dev_by_median: sk/|median(k)|),
   standard deviation by mode (c_std_dev_by_mode: sk/|mode(k)|),
   skewness (c_skewness: skewness(k)),
   kurtosis (c_kurtosis: kurtosis(k)),
   mean curvature (c_mean: mean(|k−<k>|)),
   range of curvature (c_range_v: range(k)),
   interquartile range (c_interquartile_range: interquartile range (k)),
   sum curvature profile squared (c_sum_curvature_profile_square: sum(k2)),
   sum curvature profile cubed (c_sum_curvature_profile_cube: sum(k3)),
   mean curvature profile squared (c_mean_curvature_profile_square: mean(k2)),
   mean curvature profile cubed (c_mean_curvature_profile_cube: mean(k3)),
   mean curvature profile raised to four (c_mean_curvature_profile_raise_to_four: mean(k4)),
   mean curvature profile raised to five (c_mean_curvature_profile_raise_to_five: mean(k5)),
   sum curvature profile minus mean power of 2 (c_sum_curvature_profile_minus_mean_pow2: sum(|k−<k>|2)),
   sum curvature profile minus mean power of 3 (c_sum_curvature_profile_minus_mean_pow3: sum(|k−<k>|3)),
   mean curvature profile minus mean power of 2 (c_mean_curvature_profile_minus_mean_pow2: mean(|k−<k>|2)),
   mean curvature profile minus mean power of 3 (c_mean_curvature_profile_minus_mean_pow3: mean(|k−<k>|3)),
   mean curvature profile minus mean power of 4 (c_mean_curvature_profile_minus_mean_pow4: mean(|k−<k>|4)),
   mean curvature profile minus mean power of 5 (c_mean_curvature_profile_minus_mean_pow5: mean(|k−<k>|5)),
   number of peaks (c_number_of_peaks: number of peaks),
   gini coefficient (c_gini_coefficient: gini coefficient (k)).

The image features may be evaluated using one or more statistical metrics. One or more feature selection processes may be used to select image features that are associated with oncogenic drivers. Non-limiting example statistical metrics are standard deviation, quadratic entropy which averages the difference between two randomly-drawn samples, Kolmogorov-Smirnov which is based on the distance between the normal distribution and the empirical distribution function of a sample, and outlier percentage (e.g., percentage of values outside the range of twice the standard deviation from the mean). In some embodiments, the selected images features may have the highest relevance to oncogenic drivers amongst the plurality of image features. The oncogenic drivers may be fusion, mutation, or unknown drivers.

In some embodiments, example selected nuclear morphology image features may comprise:
basic morphology features, such as:
   area of the nucleus (area),
   the ratio of pixels in the nucleus to pixels in a bounding box, either in a total field or view or a selected area of a field of view (extent),
   the ratio of pixels in the nucleus to pixels of a convex hull (solidity),
   Hu moments (a certain particular weighted average, a.k. a. “moment,” of the image pixels' intensities, i.e., translation, rotation, and scale-invariant moments) (moments_hu0),
   weighted Hu moments (weighted_moments_hu0, weighted_moments_hu1, weighted_moments_hu2);
two-dimensional shape features, such as the two-dimensional shape perimeter-to-surface ratio (original_shape2D_PerimeterSurfaceRatio);
first order statistical image features, which describe the distribution of pixel intensities within the image region defined by the mask through commonly used and basic metrics, such as:
   first-order 90^{th} percentile (original_firstorder_90Percentile),
   first-order minimum (original_firstorder_Minimum),
   first-order entropy, which specifies the uncertainty or randomness in the image values (original_firstorder_Entropy);
gray-level co-occurrence matrix (GLCM) (describes the second-order joint probability function of an image region constrained by the mask) image features, such as:
   GLCM inverse difference (original_glcm_Id),
   GLCM contrast (original_glem_contrast),
   GLCM joint entropy (original_glcm_JointEntropy),
   GLCM sum entropy (original_glcm_SumEntropy);
gray-level dependence matrix (GLDM) (quantifies gray level dependencies in an image, wherein a gray level dependency is defined as the number of connected pixels within a specified distance that are dependent on the center pixel) image features, such as:
   GLDM gray level dependence entropy (original_gldm_DependenceEntropy),
   GLDM dependence non uniformity normalized (DNUN) which measures the similarity of dependence through the image and is normalized (original_DependenceNonUniformityNormalized),
   small dependence emphasis (SDE) which measures the distribution of small dependencies (original_gldm_SmallDependenceEmphasis);
gray-level run length matrix (GLRLM) (quantifies gray level runs, which are defined as the length in number of pixels, of consecutive pixels that have the same gray level value) image features, such as:
   GLRLM long-run emphasis (LRE) (original_glrlm_LongRunEmphasis),
   GLRLM long-run low gray-level emphasis (original_glrm_LongRunLowGrayLevelEmphasis),
   GLRM run length nonuniformity (original_glrm_RunLengthNonuniformity),
   GLRM run entropy (original_glrm_RunEntropy);
curvature image features, such as:
   mean curvature (c_mean),
   median curvature (c_median),
   25^{th} percentile curvature (c_percentile_25),
   75^{th} percentile curvature (c_percentile_75),
   above 75^{th} percentile of the mean curvature (c_mean_above_percentile_75),
   15% trimmed mean curvature (c_trimmed_mean_15_percent),
   25% trimmed mean curvature (c_trimmed_mean_25_percent),
   interquantile range curvature (c_interquantile_range),
   gini coefficient of a curvature (c_gini_coefficient); and
advanced nucleus morphology features, such as:
   radius of an ellipse-shaped nucleus (ellipse_R_index),
   major axis of an ellipse-shaped nucleus (ellipse_MA_index)
   convexity perimeter of a nucleus, which measures the perimeter of curvature (convexity_perimeter),
   normalized number of connected components that remain when a shape is subtracted from a convex hull (Ncce_index).

In some instances, predicting or determining that an actionable gene fusion is present may be based, at least in part, on the detection of extracellular mucin. Excess extracellular mucin is reported to be indicative of fusion status and the disclosed methods for gene fusion status prediction may substantiate these findings. In some instances, the digital pathology image processing system 110 may predict gene fusion status in detail, identify differences between, e.g., resections and biopsies, determine precise segmentation of area, perform coarse detection of image patches containing extracellular mucin, and transition from tumor area detection to actual gene fusion status prediction. As an example and not by way of limitation, in some instances transitioning from tumor area detection to actual gene fusion status prediction may comprise determining a fraction of mucin detected versus tissue, or determining a fraction of mucin detected versus tumor.

In some instances, the digital pathology machine learning model may be generically applicable across different tumor types. Therefore, the digital pathology image processing system 110 may be used to identify and predict pan-tumor or tumor-agnostic actionable gene fusion based on the use of the digital pathology machine learning model. For example, the performance of a digital pathology image processing system 110 comprising a digital pathology machine learning model trained on ALK fusion or on ROS1 fusion, respectively, was the same. As another example, the signal for NTRK fusions may sort with ALK, ROS1, and RET. For instance, even though a digital pathology machine learning model was trained without using NTRK-based training data, it was able to identify NTRK fusions with the same accuracy as it had for detection of ROS1 fusions in experiments to test the methods disclosed herein. The general applicability of the digital pathology machine learning model may suggest that the underlying image patch features used for prediction are consistent across different gene fusions as well as across different tumor types.

In particular instances, the digital pathology image processing system 110 may indicate the occurrence of gene fusion to a pathologist as, for example, a comparison between a fusion positive slide image and the same field of view from the slide with an overlaid heatmap of gene fusion prediction. When comparing the two, the pathologist may thus see how a tumor detection algorithm in some instances of the methods disclosed herein rejected the image patches containing no tumor. In addition, confidence metric(s) for the prediction of gene fusion (as depicted, for example, by the intensity of the heatmap) may vary across the tumor area. In some instances, for example, confidence metrics may be highest in areas with signet ring cells.

Returning to FIG. 4, at step 450 the digital pathology image processing system 110 may generate, based on the detected occurrence of gene fusion with respect to the cancer cells, a subject prediction for the subject, wherein the subject prediction comprises a prediction of applicability of one or more treatment regimens for the subject. The digital pathology image processing system 110 may output, e.g., via a graphical user interface, the subject prediction. As an example and not by way of limitation, the digital pathology image processing system 110 may output a treatment regimen assessment. The digital pathology image processing system 110 may generate a recommendation associated with use of the one or more treatment regimens. For instance, the assessment may be that a given patient is likely to have a gene fusion. As a subsequent or further step, digital pathology image processing system 110 may prompt a recommendation of performing a follow-up molecular test, such as a next-generation sequencing assay. In some instances, one or more steps of the method depicted in FIG. 4 may be repeated where appropriate. Although this disclosure describes and illustrates particular steps of the method of FIG. 4 as occurring in a particular order, this disclosure contemplates any suitable steps of the method of FIG. 4 occurring in any suitable order. Moreover, although this disclosure describes and illustrates an example method for detecting gene fusion (or other gene alterations), including the particular steps of the method depicted in FIG. 4, this disclosure contemplates any suitable method for detecting gene fusion, including any suitable steps, which may include all, some, or none of the steps of the method of FIG. 4, where appropriate. Furthermore, although this disclosure describes and illustrates particular components, devices, or systems carrying out particular steps of the method of FIG. 4, this disclosure contemplates any suitable combination of any suitable components, devices, or systems carrying out any suitable steps of the method of FIG. 4.

The methods and systems disclosed herein may have a technical advantage of using easily accessible and less expensive material for analysis than corresponding molecular tests. In some instances, for example, a section of the biological sample may be stained with one or more stains. The digital pathology image processing system may be used to scan, e.g., hematoxylin and eosin (H&E) stained slides; the original tissue specimen slides are readily available for use in new or follow-up diagnostic analyses. By contrast, a molecular test may require cutting into the tissue block to sacrifice some tissue for use in sequencing, which would result in consumption of diagnostic tissue material. As can be seen, no tissue would be destroyed when using a digital pathology machine learning model to analyze image data. In some instances, one may use the digital pathology image of a primary diagnostic slide for analysis without requiring extra slides. In some instances, a subject prediction may be generated based on further analysis of one or more additional digital pathology images. In some instances, each of the one or more additional digital pathology images may depict an additional section of the biological sample from the subject. In some instances, the analysis may comprise determining whether each of the one or more additional digital pathology images comprises a depiction of an occurrence of gene fusion with respect to the cancer cells, and combining the determination for each of the one or more additional digital pathology images. In some instances, after making a diagnosis based on an analysis of the image for an H&E stained specimen slide, one may require additional unstained specimen slides (e.g., at least 5, 6, 7, 8, 9, 10 or more than 10 unstained specimen slides) to be sacrificed to perform the molecular test.

The disclosed methods have another technical advantage in terms of ease-of-use. One may scan the pathology specimen slide and input the scanned image, or image patch data derived therefrom, to the digital pathology machine learning model. The digital pathology machine learning model may then be used to make a prediction of whether or not a gene alteration, e.g., a gene fusion, is present in the biological sample. In some instances, the process may not require any annotation by a pathologist. In some instances, the pathologist may only have to correctly identify the slide as a target tumor type, e.g., lung adenocarcinoma. The methods disclosed herein may have another technical advantage in terms of efficiency. As an example, and not by way of limitation, the prediction of gene fusion may be completed in a matter of minutes, hours, or days, e.g., in less than 60 minutes, less than 50 minutes, less than 40 minutes, less than 30 minutes, less than 25 minutes, less than 20 minutes, less than 15 minutes, or less than 10 minutes.

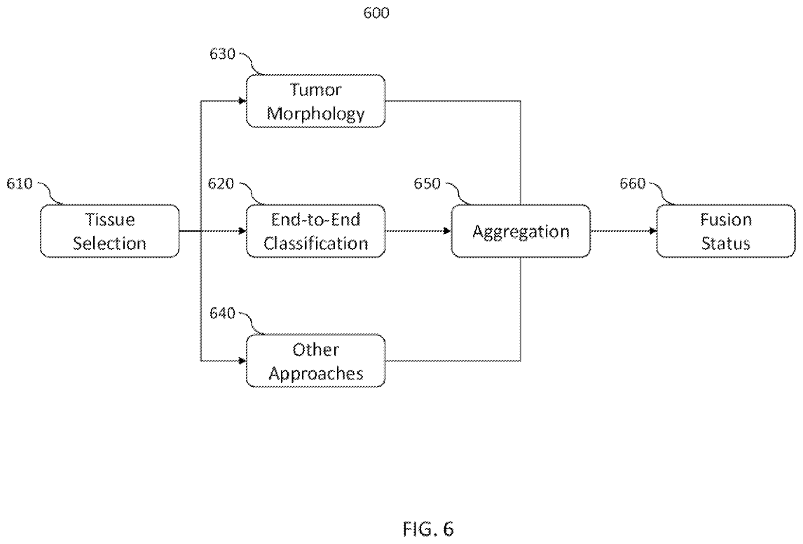

FIG. 6 provides an exemplary workflow diagram for a process 600 for detecting gene fusion in a biological sample, e.g., a tissue specimen. The process 600 may start with tissue selection 610. In tissue selection 610, the digital pathology image processing system 110 may perform quality control and/or tumor detection. In some instances, quality control and/or tumor region detection may comprise performing supervised classification tasks. For such tasks, image patch-level accuracy may be sufficient and the digital pathology image processing system 110 may use, e.g., one binary classifier per task. The results of tissue selection 610 may then be provided as input to an end-to-end classification step 620. In some instances, the end-to-end classification 620 may be based on one or more of image patch-based classification or multi-instance learning (MIL) classification techniques as described above. As part of the end-to-end classification 620, generating a label for each of a plurality of image patches may be performed by one or more machine-learning models. In some instances, the digital pathology image processing system 110 may train the one or more machine-learning models based on a plurality of training data comprising, e.g., one or more labeled depictions of a tumor region or tumor nest structure and one or more labeled depictions of other histologic or clinical features.

While the end-to-end classification 620 is being performed, the digital pathology image processing system 110 may perform tumor morphology analysis 630. In some instances, generating the label for each of a plurality of image patches may be based on tumor morphology. The tumor morphology analysis 630 may comprise an analysis to identify one or more of a signet ring cell, a hepatoid cell, extracellular mucin, a tumor growth pattern, or tumor heterogeneity. In some instances, growth pattern analysis may be helpful for gene fusion detection. As an example and not by way of limitation, lung adenocarcinomas may present with a number of growth patterns and with varying proportions of each. As another example and not by way of limitation, in some instances solid and cribriform patterns may be associated with gene fusions. In some instances, the digital pathology image processing system 110 may determine the influence of sample collection type (e.g., resection versus biopsy) on growth patterns. Since growth patterns are often large and homogeneous regions, image patch-level classification may be sufficiently accurate. In some instances, signet ring cell detection and hepatoid cell detection may both be associated with the presence of gene fusions. To detect such cells of interest, the digital pathology image processing system 110 may rely on object detection and localization. In some instances involving cell of interest detection, the digital pathology image processing system 110 may determine a relationship between detected cells and fusion status, e.g., based on the number or type of cells detected. In some instances, the digital pathology image processing system 110 may further perform fine-grained localization or patch-level detection of cells. The digital pathology image processing system 110 may also use other approaches 640 for gene fusion detection. As an example and not by way of limitation, in some instances the digital pathology image processing system 110 may identify nuclear pleomorphism from the digital pathology image and measure the identified nuclear pleomorphism. Correspondingly, in some instances determining that the digital pathology image may comprise a depiction of the occurrence of gene fusion may be further based on the measured nuclear pleomorphism. The digital pathology image processing system 110 may then perform aggregation 650 on the results from tumor morphology analysis 630, end-to-end classification 620, and other approaches 640. The aggregated results may be used to predict the fusion status 660 for the tissue specimen. In some instances, the fusion status prediction may comprise a weakly-supervised classification task (e.g., in which slide-level labels may be available). In some instances, the digital pathology image processing system 110 may use a multi-instance learning (MIL) approach to classify a plurality of image patches. In some instances, the digital pathology image processing system 110 may use a simplified strategy comprising the assignment of a slide label to all image patches. In particular instances, determining that the digital pathology image comprises a depiction of the occurrence of gene fusion with respect to the cancer cells may be further based on a weighted combination of the labels generated for each image patch. As an example and not by way of limitation, in some instances the digital pathology image processing system 110 may use a binary classifier to classify image patches and then determine a slide-level prediction by combining (e.g., averaging) all image patch predictions.

In particular instances, the digital pathology image processing system 110 may output, via a graphical user interface, the subject prediction. In some instances, the graphical user interface may comprise a graphical representation of the digital pathology image. In some instances, the graphical representation may comprise an indication of the label generated for each of a plurality of image patches and a predicted level of confidence associated with the respective label. In some instances, the output of the digital pathology image processing system 110 may also comprise other information as follows. As an example and not by way of limitation, the digital pathology image processing system 110 may output a rhetoric assessment. The digital pathology image processing system 110 may generate a recommendation associated with use of one or more treatment regimens for the subject or patient from which the biological sample was derived. For instance, the assessment may be that a sample from a given subject or patient is likely to have a gene fusion, so confirmation by a follow-up molecular assay is recommended. As another example and not by way of limitation, the digital pathology image processing system 110 may output a negative result, i.e., that there is no gene fusion predicted or detected. As yet another example and not by way of limitation, the digital pathology image processing system 110 may output “insufficient data for analysis”. For example, “insufficient data for analysis” may be due to either the tumor size or the pathology slide preparation (e.g., the tumor specimen was too small and/or the pathology slide quality was hampered by tissue handling artifacts). For instance, the microtome blade used in cutting a tissue section may produce a series of parallel tears across the tissue section placed on the slide. These types of sample processing artifacts may prevent the digital pathology machine learning model(s) used to analyze the pathology slide image from making an accurate prediction.

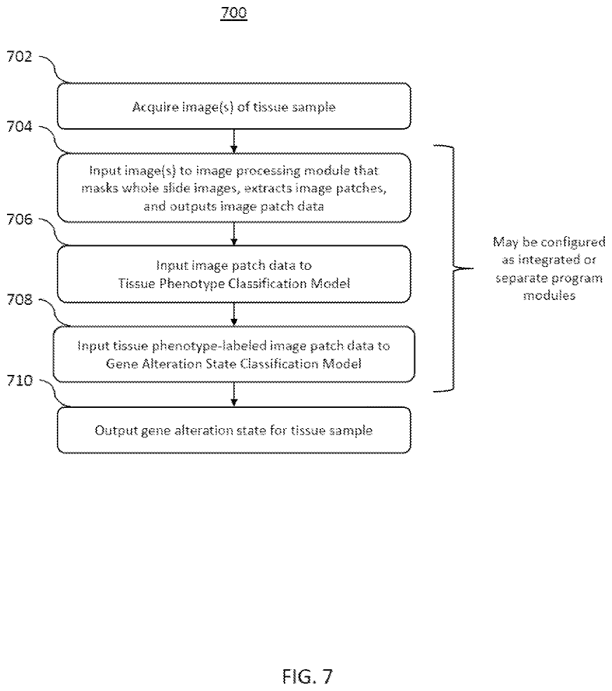

FIG. 7 provides a non-limiting illustration of a workflow 700 for performing image-based analysis and determination of gene alteration states in tissue samples according to one implementation of the disclosed methods and systems. A user, e.g., a pathologist or pathology lab technician, prepares a tissue sample and acquires one or more images of the specimen, 702. The image(s) are uploaded to a computer system configured to execute the code in one or more program modules which sequentially perform image processing to mask the whole slide image(s) and extract image patches, 704. The image patch data is then input into a trained tissue phenotype classification model, 706, which classifies the image patch data according to tissue phenotype class and/or image feature cluster. The labeled image patch data output by the tissue phenotype classification model is then input into a trained gene alteration state classification model, 708, which classifies the labeled image patch data and outputs a determination of gene alteration state for the tissue sample, 710.

The disclosed machine learning-based methods and systems for inferring gene alteration state from pathology slide images may be deployed in individual pathology labs, for example, as stand-alone workstations or integrated with a clinical laboratory information management (LIMS) system. In some instances, the disclosed machine learning-based methods and systems may be deployed as part of, for example, a distributed network of local and/or remote pathology labs that procure and prepare tissue specimens, which can then be imaged and the images uploaded to a computer server, where the computer server is optionally linked to the Internet. The disclosed machine learning-based methods (using one or more machine learning models, which optionally may be deployed in a distributed network of computer systems or computer servers (e.g., in the cloud)) may then be used to predict gene alteration state in a given tissue sample to provide enhanced decision making insight to patients and/or healthcare providers (via accelerated feedback) in advance of the physical sample optionally being shipped to a genotyping or sequencing lab for confirmation of the determined gene alteration state.

In some instances, the disclosed methods and systems may be used for selecting a treatment for an individual having cancer. Following the collection, preparation, and imaging of one or more tissue samples from the individual patient, the disclosed methods and systems may be used to: a) detect a gene alteration state from the one or more pathology image of the tissue sample derive from the individual, wherein the gene alteration state is detected according to any of the methods described herein; and/or b) select a treatment based on the detected gene alteration state. In some instances, the cancer may be lung cancer. In some instances, the lung cancer may be lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma. In some instances, the gene alteration state may comprise a mutation in any gene associated with a cancer, e.g., a lung cancer such as lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma. In some instances, the gene alteration state may comprise, e.g., a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene, or any combination thereof. Examples of HRR pathway genes include, but are not limited to, BRCA1, BRCA2, CHEK2, ATM, PALB2, FANCA, and RAD51D.

In some instances, the disclosed methods and systems may be used for selection, initiation, adjustment, or discontinuation of a treatment of an individual patient. Following the collection, preparation, and imaging of one or more tissue samples from the individual patient, the disclosed methods and systems may be used, for example, to: a) detect a gene alteration state from a pathology image of a tissue sample derive from the individual, wherein the gene alteration state is detected according to any of the methods described herein; b) select a treatment based on the detected gene alteration state; and c) treat the individual by administering the selected treatment to the individual. In some instances, the cancer may be lung cancer. In some instances, the lung cancer may be lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma. In some instances, the gene alteration state may comprise a mutation in any gene associated with a cancer, e.g., a lung cancer such as lung adenocarcinoma, lung adenosquamous cell carcinoma, lung squamous cell carcinoma, lung large cell carcinoma, lung large cell neuroendocrine carcinoma, lung carcinosarcoma, lung sarcomatoid carcinoma, or lung small cell carcinoma. In some instances, the gene alteration state may comprise a mutation in an epidermal growth factor receptor (EGFR) gene, an anaplastic lymphoma kinase (ALK) fusion oncogene, a receptor tyrosine kinase (ROS1) oncogene, a kinesin family 5B (KIF5B) gene, a receptor tyrosine kinase (RET) oncogene, a neurotrophic tyrosine receptor kinase (NTRK) oncogene, a BRCA1 gene, a BRCA2 gene, an erb-B2 receptor tyrosine kinase 2 (ERBB2) gene, a B-Raf (BRAF) gene, a Kirsten rat sarcoma viral (KRAS) oncogene, a MET proto oncogene, a serine/threonine kinase 11 (STK11) gene, a homologous recombination repair (HRR) pathway gene, or any combination thereof. Examples of HRR pathway genes include, but are not limited to, BRCA1, BRCA2, CHEK2, ATM, PALB2, FANCA, and RAD51D.

In some instances, for example, the gene alteration state may comprise a mutation in an epidermal growth factor receptor (EGFR) gene, and the selected treatment may comprise a kinase inhibitor, a small molecule drug, an antibody or antibody fragment, or a cellular immunotherapy that inhibits EFGR activity. In this case, examples of suitable kinase inhibitors include, but are not limited to, a multi-specific kinase inhibitor, a specific tyrosine kinase inhibitor, a specific EGFR inhibitor, or a dual EGFR/ERBB inhibitor that inhibits EGFR activity.

In some instances, the gene alteration state may comprise a mutation in an anaplastic lymphoma kinase (ALK) fusion oncogene, and the selected treatment may comprise a specific kinase inhibitor that inhibits ALK activity. In this case, examples of suitable specific kinase inhibitors include, but are not limited to, crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), or any combination thereof.

In some instances, the gene alteration state may comprise a mutation in a receptor tyrosine kinase (ROS1) oncogene, and the selected treatment may comprise a specific kinase inhibitor that inhibits ROS1 activity. In this case, an example of suitable specific kinase inhibitors includes, but is not limited to, entrectinib (also known as RXDX-101 or NMS-E628).

In some instances, the gene alteration state may comprise a mutation in a receptor tyrosine kinase (RET) oncogene, and the selected treatment may comprise a specific kinase inhibitor that inhibits RET activity. In this case, examples of suitable specific kinase inhibitors include, but are not limited to, selpercatinib, pralsetinib, TPX-0046, or any combination thereof.

In some instances, the gene alteration state may comprise a mutation in a neurotrophic tyrosine receptor kinase (NTRK) oncogene, and the selected treatment may comprise a specific NTRK inhibitor that inhibits NTRK activity. In this case, examples of suitable specific NTRK inhibitors include, but are not limited to, larotrectinib, entrectinib, LOXO-195, danusertib (PHA-739358), lestaurtinib, AZ-23, PHA-848125, CEP-2563, K252a, KRC-108, or any combination thereof.

In some instances, the gene alteration state may comprise a mutation in a homologous recombination repair (HRR) pathway gene, and the selected treatment may comprise a platinum-based chemotherapy or a poly-ADP ribose polymerase (PARP) inhibitor that inhibits the activity of a mutated HRR pathway protein. In this case, examples of suitable PARP inhibitors include, but are not limited to, olaparib, niraparib, rucaparib, or any combination thereof.

Additional examples of targeted therapies that may be selected and administered in response to a determination of a specific gene alteration state using the methods disclosed herein are described in PCT International Application Nos. PCT/US2012/051978, PCT/US2013/068604, and PCT/US2013/068457, which are incorporated herein by reference in their entirety.

In some instances, any of the methods disclosed herein may further comprise performing one or more additional procedures based on the determined gene alteration state. For example, in some instances, one or more additional diagnostic tests may be performed, e.g., to confirm a diagnosis of a disease such as cancer. In some instances, a treatment may be selected for an individual patient (e.g., a cancer patient) based on the determined gene alteration state. In some instances, the dosage for a treatment may be adjusted.

As another example, in some instances the one or more procedures may comprise performing one or more genomic profiling assays, which may be used, e.g., select a treatment for, adjust the dosage of a treatment for, or to monitor progression of a cancer in an individual having cancer.

In some instances, the one or more genomic profiling assays may comprise obtaining a nucleic acid sample from a patient from which the tissue sample was derived, and sequencing the nucleic acid to perform a molecular profiling test. The nucleic acid sample may comprises a deoxyribonucleic acid (DNA) sample or a ribonucleic acid (RNA) samples. Examples of suitable deoxyribonucleic acid (DNA) samples include, but are not limited to, tissue-derived DNA, cell-free DNA (cfDNA), circulating tumor DNA (ctDNA), or mitochondrial DNA. Examples of suitable ribonucleic acid (RNA) samples include, but are not limited to, messenger RNA (mRNA), ribosomal RNA (rRNA), transfer RNA (tRNA), or mitochondrial RNA. In some instances, the nucleic acid sample is derived from a tissue sample, a blood sample, a urine sample, a saliva sample, a biopsy sample, or a liquid biopsy sample from a subject or patient. Examples of molecular profiling tests that may be performed as a follow-up to determination of gene alteration state from analysis of a pathology slide image include, but are not limited to, a comprehensive genomic profiling (CGP) test, a gene expression profiling test, a cancer hotspot panel test, a DNA methylation test, a DNA fragmentation test, an RNA fragmentation test, or any combination thereof. The results of the molecular profiling test may be used, for example, to select or adjust a treatment for an individual having cancer. In some instances, the disclosed methods may further comprise treating the individual having cancer.

In some instances, the one or more additional procedures may comprise, for example, performing a follow-up screening test. For example, the follow-up screening test may comprise a colonoscopy, and follow-up colonoscopies may be recommended at more frequent intervals (e.g., every 6 months, 1 year, 2, years, 3 years, or 4 years, instead of every 5 years) for a given patient depending on the determined gene alteration state of a tissue sample from the patient.

In some instances, the disclosed methods may further comprise displaying a result of the molecular profiling test on a display device, generating a report for the molecular profiling test, transmitting a report for the molecular profiling test to a healthcare provider through a computer network, or transmitting a report for the molecular profiling test to a healthcare provider over the Internet.

### Tissue Samples

As noted above, the disclosed methods and systems may be applicable to any of variety of tissue samples, e.g., solid tissue samples or soft tissue samples. Examples include, but are not limited to, connective tissue, muscle tissue, nervous system tissue, and epithelial tissue. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

Tissue samples may be collected using any of a variety of techniques known to those of skill in the art including, but not limited to, direct collection, biopsy, surgical resection, etc. Examples of specific biopsy techniques include, but are not limited to, bone marrow biopsies, endoscopic biopsies, needle biopsies, skin biopsies, surgical biopsies, etc.

In some instances, the tissue sample may be processed as part of preparation of a pathology slide and imaging. Examples of tissue preparation steps include, but are not limited to, tissue fixation (e.g., using 10% neutral buffered formalin to prevent tissue autolysis and putrefaction), trimming and transfer to labeled tissue cassettes (for storage in, e.g., formalin, until being processed), tissue processing (e.g., dehydration (e.g., by immersion in increasing concentrations of alcohol to remove water and formalin), clearing (e.g., using an organic solvent such as xylene to remove alcohol and allow infiltration with, e.g., paraffin wax), and embedding (e.g., infiltration with an embedding agent such as paraffin wax)), sectioning (e.g., slicing into thin tissue sections using a microtome), and staining (e.g., using a fluorescently-labeled antibody or histochemical stain such as hematoxylin or eosin to enhance contrast and make tissue structure more visible).

### Tissue Phenotype Classes

As noted above, in some instances, pathology images and/or image patches derived therefrom may be annotated by a pathologist to indicate different tissue phenotype classes present in the image or image patch. In some instances, tissue phenotype classes may comprise any of a variety of normal and/or abnormal tissue morphology and/or tissue histology classes known to those of skill in the art. Examples of tissue phenotypes may include, but are not limited to, tissue structure, shape, color, pattern, texture, cell type(s) present, cell morphologies, extracellular matrix, and the like.

In some instances, tissue phenotype classes may comprise a feature or set of features that are not visibly identifiable by a pathologist. For example, in some instances, tissue phenotype classes may be defined by clusters of image features extracted from image patches using, e.g., a machine learning-based feature extraction approach.

In some instances, image patch data derived from one or more pathology images may be annotated, labeled, or sorted into, 1, 2, 3, 4, 5, 6, 7, 8, 9, 10, 11, 12, 13, 14, 15, 16, 17, 18, 19, 20, or more than 20 different tissue phenotype classes.

### Disease States

The disclosed methods and systems may be applicable to determination of gene alteration states of relevance to any of a variety of disease states known to those of skill in the art. Examples include, but are not limited to, skin cancer (e.g., basal cell skin cancer, squamous cell skin cancer, melanoma), lung cancer, prostate cancer, breast cancer, colorectal cancer, kidney (renal) cancer, urinary bladder cancer, non-Hodgkin's lymphoma, thyroid cancer, endometrial (uterus) cancer, pancreatic cancer, and the like.

### Gene Alteration States

In some instances, the disclosed methods and systems may be applicable to identification of specific gene alteration states that are associated with one or more disease states. In some instances, they may be used to identify gene alterations states in a tissue sample that are associated with one, two, three, four, five, or more than five distinct disease states.

In some instances, the disclosed methods and systems are configured to identify gene alteration states that correspond to the presence or absence of at least one genetic mutation in at least one gene. For example, in some instances, the disclosed methods and systems may be used to identify one, two, three, four, five, or more than five genetic mutations in each of one, two, three, four, five, or more than five genes respectively. As used herein, a genetic mutation (or simply, a mutation) may refer to a point mutation, insertion, deletion, copy number variation (CNV), rearrangement, fusion, tumor mutational burden, microsatellite instability status, homologous recombination deficiency, or any combination thereof.

Examples of gene alteration states that may be detected using the disclosed methods and systems include, but are not limited to, mutations in, e.g., the epidermal growth factor receptor (EGFR) gene, the anaplastic lymphoma kinase (ALK) fusion oncogene, the receptor tyrosine kinase (ROS1) oncogene, the kinesin family 5B (KIF5B) gene, the receptor tyrosine kinase (RET) oncogene, the neurotrophic tyrosine receptor kinase (NTRK) oncogene in the case of lung cancer tissue, mutations in the BRCA1 or BRCA2 genes in the case of breast cancer, the erb-B2 receptor tyrosine kinase 2 (ERBB2) gene, the B-Raf (BRAF) gene, the Kirsten rat sarcoma viral (KRAS) oncogene, the MET proto oncogene, the serine/threonine kinase 11 (STK11) gene, and the like.

### Pathology Imaging Techniques

The disclosed methods and systems may be utilized with images, e.g., whole slide pathology images, of tissue samples that have been acquired using any of a variety of microscopy imaging techniques known to those of skill in the art. Examples include, but are not limited to, bright-field microscopy, dark-field microscopy, phase contrast microscopy, differential interference contrast (DIC) microscopy, fluorescence microscopy, confocal microscopy, confocal laser microscopy, super-resolution optical microscopy, scanning or transmission electron microscopy, and the like.

### Image Processing & Image Processing Methods

In some instances, the disclosed methods may comprise one or more image processing steps to process whole slide pathology images and/or image patches derived therefrom. Image processing may be performed prior to performing machine learning-based analysis on images or image patches, and/or at one or more intermediate steps of the machine learning-based analysis. Examples of image processing operations that may be performed include, but are not limited to, image exposure correction (e.g., white balance adjustment, contrast adjustment), flat-field correction, aberration correction, noise removal, masking (e.g. binary masking) to separate tissue from non-tissue regions of the whole slide image and/or to extract image patches, object and/or structure identification, or any combination thereof.

Any of a variety of image processing methods known to those of skill in the art may be used for image processing/pre-processing. Examples include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation methods (e.g., intensity thresholding, intensity clustering methods, intensity histogram-based methods, etc.), feature and pattern recognition methods (e.g., the generalized Hough transform for detecting arbitrary shapes, the circular Hough transform, etc.), and mathematical analysis methods (e.g., Fourier transform, fast Fourier transform, wavelet analysis, auto-correlation, etc.), or any combination thereof.

### Image Patch Extraction

In some instances, image patches may be extracted from whole slide pathology images using, e.g., using masking or other image processing techniques such as those described above. In some instances, 5, 10, 15, 20, 25, 30, 40, 50, 60, 70, 80, 90, 100, 200, 400, 600, 800, 1000, 1500, 2000, 2500, 3000, 3500, 4000, 4500, 5000, or more than 5000 image patches may be extracted from each whole slide pathology image. In some instances, the number of image patches extracted from each whole slide pathology image may have any value within the range of values described in this paragraph, e.g., 1,224 image patches.

In some instances, the image patches extracted from a tissue image may be of different sizes. In some instances, the image patches extracted from a tissue image may all be of the same size. In some instances, the image patches extracted from a tissue image may be of a predetermined size. In some instances, the image patches may all be of the same size when training a machine learning model for a particular classification application in order to ensure that the image patch patterns processed by the model are consistent in terms of, e.g., field of view, and to ensure that the model's weights and the patterns learned are meaningful. In some instances, image patch size may be varied from experiment to experiment or from application to application. In some instances, image patch size can be considered a tunable parameter during the training process.

In some instances, the size of the image patches may range from 10 pixels to 10⁷ pixels. In some instances, the size of the image patches may be at least 10 pixels, at least 100 pixels, at least 10³ pixels, at least 10⁴ pixels, at least 10⁵ pixels, at least 10⁶ pixels, or at least 10⁷ pixels. In some instances, the size of the image patches may be at most 10⁷ pixels, at most 10⁶ pixels, at most 10⁵ pixels, at most 10⁴ pixels, at most 10³ pixels, at most 100 pixels, or at most 10 pixels. Any of the lower and upper values described in this paragraph may be combined to form a range included within the present disclosure, for example, in some instances the size of the image patches may range from about 100 pixels to about 105 pixels. Those of skill in the art will recognize that the size of the image patches may have any value within this range, e.g., about 2.8×10³ pixels.

In some instances, image patch size (or a range of image patch sizes) is determined by the input patch size expected by the machine learning model, as well as by other image patch extraction considerations (e.g., the need to ensure that a sufficient number of image patches can be extracted from a slide, or the need to ensure that the image patches are not so large that it makes computation using a machine learning model difficult and/or computationally costly). The extracted image patch sizes may sometimes be larger than the image patches passed through the machine learning model. For example, this is sometimes done to maintain a larger field-of-view, e.g., by extracting a larger image patch (say 1024×1024×3 pixels for color images, or 1024×1024×1 pixels for grayscale images) and then down-sampling the image patch's resolution (e.g., to 299×299×3 pixels for color images, or 299×299×1 pixels for grayscale images) in order to provide the machine learning model with more computationally-efficient inputs.

In some instances, the image patches may be of a square or rectangular shape, e.g., 100 pixels×100 pixels, or 10 pixels×1000 pixels. In some instances, the image patches may be of irregular shape.

In some instances, image patches may be extracted from 1, 2, 3, 4, 5, 6, 7, 8, 9, 10, or more than 10 images of the same tissue sample.

### Machine Learning Methods

In some instances of the disclosed methods, the method may comprise the use of one or more machine learning methods and/or statistical analysis methods to perform the pre-processing of images (e.g., image segmentation to extract image patches and/or image feature extraction from the image patches) in addition to subsequently performing tissue phenotype classification and gene alteration state classification.

Any of a variety of machine learning models may be used in implementing the disclosed methods. For example, the machine learning models(s) employed may comprise a supervised learning model, an unsupervised learning model, a semi-supervised learning model, a deep learning model, etc., or any combination thereof.

### Supervised Learning Models

In the context of the present disclosure, supervised learning models are models that rely on the use of a set of labeled training data to infer the relationship between a set of input data (e.g., image patch data) and a classification of the input data into to a specified set of user-specified classes (e.g., tissue phenotype class). The training data used to “teach” the supervised learning model comprises a set of paired training examples, e.g., where each example comprises an image patch and the tissue phenotype classification of the given image patch. Examples of supervised learning models include support vector machines (SVMs), artificial neural networks (ANNs), etc.

### Unsupervised Learning Models

In the context of the present disclosure, unsupervised learning models are models used to draw inferences from training datasets consisting of image feature datasets that are not paired with labeled tissue phenotype classification data. One example of a commonly used unsupervised learning models is cluster analysis, which is often used for exploratory data analysis to find hidden patterns or groupings in multi-dimensional data sets. Other examples of unsupervised learning models include, but are not limited to, artificial neural networks, association rule learning models, etc.

### Semi-Supervised Learning Models

In the context of the present disclosure, semi-supervised learning models are models that make use of both labeled and unlabeled image patch data for training (typically using a relatively small amount of labeled data with a larger amount of unlabeled data).

### Artificial Neural Networks and Deep Learning Models

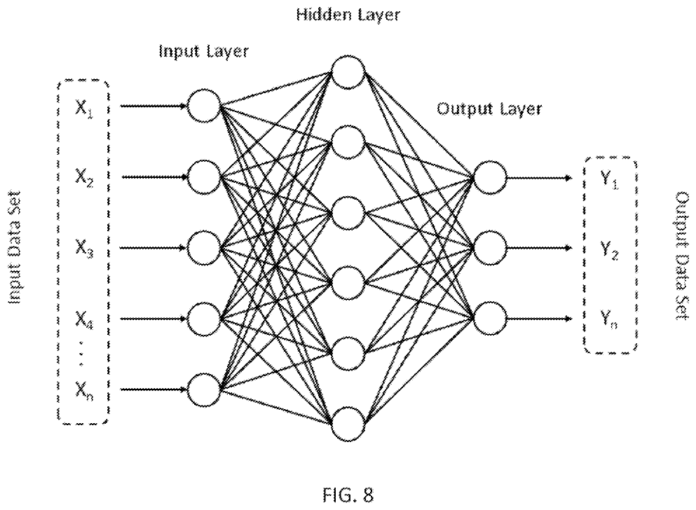

In the context of the present disclosure, artificial neural networks (ANNs) are models which are inspired by the structure and function of the human brain. Artificial neural networks comprise an interconnected group of nodes organized into multiple layers. For example, the ANN architecture may comprise at least an input layer, one or more hidden layers, and an output layer (FIG. 8). Deep learning models are large artificial neural networks comprising many hidden layers of coupled “nodes” between the input layer and output layer that may be used, for example, to map image patch data or image feature data to tissue phenotype classification decisions.

The ANN may comprise any total number of layers, and any number of hidden layers, where the hidden layers function as trainable feature extractors that allow mapping of a set of input data to a preferred output value or set of output values. Each layer of the neural network comprises a number of nodes (or “neurons”). A node receives input that comes either directly from the input data (e.g., image patch data or image feature data derived from image patch data) or from the output of nodes in previous layers, and performs a specific operation, e.g., a summation operation.

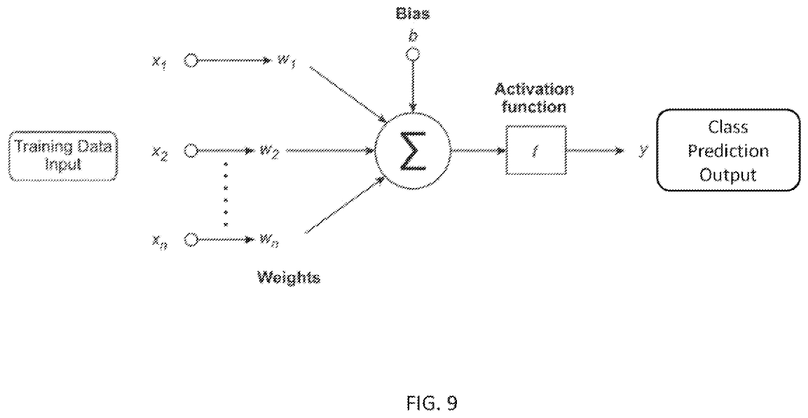

In some cases, a connection from an input to a node is associated with a weight (or weighting factor). In some cases, the node may, for example, sum up the products of all pairs of inputs, Xi, and their associated weights, Wi (FIG. 9). In some cases, the weighted sum is offset with a bias, b, as illustrated in FIG. 9. In some cases, the output of a neuron may be gated using a threshold or activation function, f, which may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., an image patch classification decision) that the ANN computes are consistent with the examples included in the training data set. The adjustable parameters of the model may be obtained using, e.g., a back propagation neural network training process that may or may not be performed using the same hardware as that used for processing images and/or performing tissue sample.

Other specific types of deep machine learning models, e.g., convolutional neural networks (CNNs) (often used for the processing of image data from machine vision systems) may also be used in implementing the disclosed methods and systems. CNN are commonly composed of layers of different types: convolution, pooling, upscaling, and fully-connected node layers. In some cases, an activation function such as rectified linear unit may be used in some of the layers. In a CNN architecture, there can be one or more layers for each type of operation performed. A CNN architecture may comprise any number of layers in total, and any number of layers for the different types of operations performed. The simplest convolutional neural network architecture starts with an input layer followed by a sequence of convolutional layers and pooling layers, and ends with fully-connected layers. Each convolution layer may comprise a plurality of parameters used for performing the convolution operations. Each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parameteric rectified linear unit, exponential linear unit, softPlus, bent identity, softExponential, Sinusoid, Sine, Gaussian, the sigmoid function and various others. The convolutional, pooling and ReLU layers may function as learnable features extractors, while the fully connected layers may function as a machine learning classifier. As with other artificial neural networks, the convolutional layers and fully-connected layers of CNN architectures typically include various adjustable computational parameters, e.g., weights, bias values, and threshold values, that are trained in a training phase as described above.

### ANN Architecture

For any of the various types of ANN models that may be used in the methods and systems disclosed herein, the number of nodes used in the input layer of the ANN (which enable input of data from, for example, sub-sampling of an image frame, a multi-dimensional image feature data set, and/or other types of input data) may range from about 10 to about 20,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 12,000, at least 14,000, at least 16,000, at least 18,000, or at least 20,000. In some instances, the number of node used in the input layer may be at most 20,000, at most 18,000, at most 16,000, at most 14,000, at most 12,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes. In some instances, the number of nodes used in the input layer may be a tunable parameter of the ANN model.

In some instances, the total number of layers used in the ANN models used to implement the disclosed methods (including input and output layers) may range from about 3 to about 1000, or more. In some instances the total number of layers may be at least 3, at least 4, at least 5, at least 10, at least 15, at least 20, at least 40, at least 60, at least 80, at least 100, at least 200, at least 300, at least 400, at least 500, at least 600, at least 700, at least 800, at least 900, or at least 1000. In some instances, the total number of layers may be at most 1000, at most 800, at most 600, at most 400, at most 200, at most 100, at most 80, at most 60, at most 40, at most 20, at most 15, at most 10, at most 5, at most 4, or at most 3. Those of skill in the art will recognize that, in some cases, the total number of layers used in the ANN model may have any value within this range, for example, 8 layers.

In some instances, the total number of learnable or trainable parameters, e.g., weighting factors, biases, or threshold values, used in the ANN may range from about 10 to about 10,000,000. In some instances, the total number of learnable parameters may be at least 10, at least 100, at least 500, at least 1,000, at least 2,000, at least 3,000, at least 4,000, at least 5,000, at least 6,000, at least 7,000, at least 8,000, at least 9,000, at least 10,000, at least 20,000, at least 40,000, at least 60,000, at least 80,000, at least 100,000, at least 250,000, at least 500,000, at least 750,00, at least 1,000,000, at least 2,500,000, at least 5,000,000, at least 7,500,000, or at least 10,000,000. Alternatively, the total number of learnable parameters may be any number less than 100, any number between 100 and 10,000, or a number greater than 10,000. In some instances, the total number of learnable parameters may be at most 10,000,000, at most 7,500,000, at most 5,000,000, at most 2,500,000, at most 1,000,000, at most 750,000, at most 500,000, at most 250,000, at most 100,000, at most 80,000, at most 60,000, at most 40,000, at most 20,000, at most 10,000, at most 9,000, at most 8,000, at most 7,000, at most 6,000, at most 5,000, at most 4,000, at most 3,000, at most 2,000, at most 1,000, at most 500, at most 100, or at most 10. Those of skill in the art will recognize that the total number of learnable parameters used may have any value within this range, for example, about 2,200 parameters.

### Autoencoders

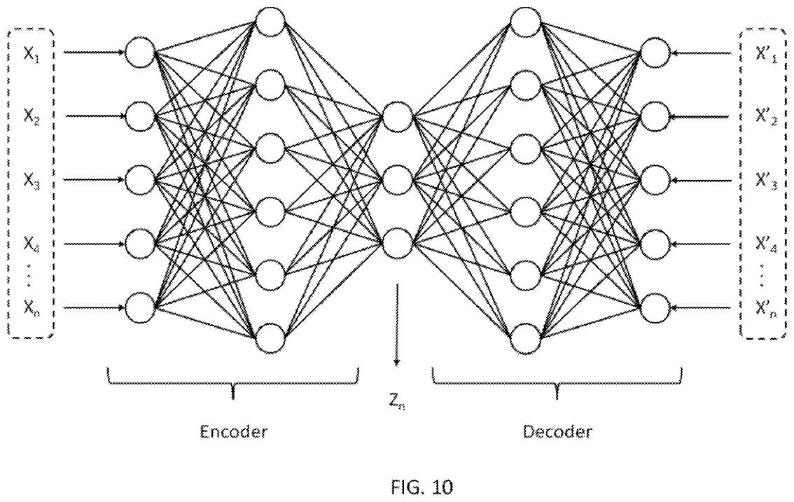

In some instances, implementation of the disclosed methods and systems may comprise the use of an autoencoder model. Autoencoders (also sometimes referred to as an auto-associator or Diabolo networks) are artificial neural networks used for unsupervised, efficient mapping of input data, e.g., image feature data, to an output value, e.g., an image cluster and/or tissue phenotype classification decision. FIG. 10 illustrates the basic architecture of an autoencoder. Autoencoders are often used for the purpose of dimensionality reduction, i.e., the process of reducing the number of random variables under consideration by deducing a set of principal component variables. Dimensionality reduction may be performed, for example, for the purpose of feature selection (e.g., selection of the most relevant subset of the image features presented in the original image feature data set) or feature extraction (e.g., transformation of image feature data in the original, multi-dimensional image space to a space of fewer dimensions as defined, e.g., by a series of feature parameters, Zₙ).

Any of a variety of different autoencoder models known to those of skill in the art may be used in the disclosed methods and systems. Examples include, but are not limited to, stacked autoencoders, denoising autoencoders, variational autoencoders, or any combination thereof. Stacked autoencoders are neural networks consisting of multiple layers of sparse autoencoders in which the output of each layer is wired to the input of the successive layer. Variational autoencoders (VAEs) are autoencoder models that use the basic autoencoder architecture, but that make strong assumptions regarding the distribution of latent variables. They use a variational approach for latent representation learning, which results in an additional loss component, and may require the use of a specific training method called Stochastic Gradient Variational Bayes (SGVB).

### Deep Convolutional Generative Adversarial Networks (DCGANs)

In some instances, implementation of the disclosed methods and systems may comprise the use of a deep convolutional generative adversarial network (DCGAN). DCGANs are a class of convolutional neural networks (CNNs) used for unsupervised learning that further comprise a generative adversarial network (GANs), i.e., they comprise a class of models implemented by a system of two neural networks contesting with each other in a zero-sum game framework. One network generates candidate images (or solutions) and the other network evaluates them. Typically, the generative network learns to map from a latent space (i.e., a representation of compressed data in which similar data points are closer together in space; latent space is useful for learning data features and for finding simpler representations of data for analysis) to a particular data distribution of interest, while the discriminative network discriminates between instances from the true data distribution and the candidate images (or solutions) produced by the generator. The generative network's training objective is to increase the error rate of the discriminative network (i.e., to “fool” the discriminator network) by producing novel synthesized instances that appear to have come from the true data distribution). In practice, a known dataset serves as the initial training data for the discriminator. Training the discriminator involves presenting it with samples from the dataset, until it reaches some level of accuracy. Typically the generator is seeded with a randomized input that is sampled from a predefined latent space (e.g., a multivariate normal distribution). Thereafter, samples synthesized by the generator are evaluated by the discriminator. Backpropagation is applied in both networks so that the generator produces better images, while the discriminator becomes more skilled at flagging synthetic images. The generator is typically a deconvolutional neural network, and the discriminator is a convolutional neural network. In some instances, implementation of the disclosed methods and systems may comprise the use of a Wasserstein generative adversarial network (WGAN), a variation of the DCGAN structure that uses a slightly modified architecture and/or a modified loss function.

### Clustering Methods

In some instances, the disclosed methods and systems may comprise the use of a clustering method to cluster image patch data according to extracted image features. Any of a variety of clustering methods known to those of skill in the art may be used. Examples of suitable clustering methods include, but are not limited to, k-means clustering methods, hierarchical clustering methods, mean-shift clustering methods, density-based spatial clustering methods, expectation-maximization clustering methods, and mixture model (e.g., mixtures of Gaussians) clustering methods.

K-means clustering methods are unsupervised machine learning methods used to partition n data points into k non-overlapping clusters such that each data point belongs to only one cluster and data points in the same cluster are characterized by, e.g., similar features, while data points in different clusters are characterized by very different features. Data points are assigned to a cluster such that the sum of the squared distances between the data points belonging to the cluster and the cluster's centroid (or arithmetic mean of all the data points that belong to that cluster) is minimized.

Hierarchical clustering methods are methods that also group data points into groups or clusters. The objective is to identify a set of clusters that characterize the original data set, where each cluster is distinct from each other cluster the data points within each cluster share broadly similar features, and each data point belongs to a single cluster. Initially, each data point is treated as a separate cluster. A distance matrix for pairs of data points is calculated, and the method then repeats the steps of: (i) identifying the two clusters that are closest together, and (ii) merging the two most similar clusters. The iterative process continues until all similar clusters have been merged.

Gaussian mixture models are probabilistic models that assume all data points in a data set may be represented by a mixture of a finite number of Gaussian distributions with unknown peak height, position, or standard deviations. The approach is similar to generalizing a k-means clustering method to incorporate information about the covariance structure of the data as well as the centers of the latent Gaussians.

### Machine Learning Training Data

The type of training data used for training a machine learning model for use in the disclosed methods and systems will depend on, for example, whether a supervised or unsupervised approach is taken as well as on the objective to be achieved. In some instances, one or more training data sets may be used to train the model(s) in a training phase that is distinct from that of the application (or deployment) phase. In some instances, training data may be continuously updated and used to update the machine learning model(s) in a local or distributed network of one or more deployed pathology image analysis systems in real time. In some cases, the training data may be stored in a training database that resides on a local computer or server. In some cases, the training data may be stored in a training database that resides online or in the cloud.

In some instances, e.g., classification of image patch data into tissue phenotype classes, the training data may comprise data derived from a series of one or more pre-processed, segmented images where each image of the series comprises an image of an individual tissue sample. In some instances, a machine learning model may be used to perform all or a portion of the pre-processing and segmentation of the series of one or more tissue sample images as well as the subsequent analysis (e.g., a tissue phenotype classification decision, or a gene alteration state determination). In some cases, the training data set may include other types of input data, e.g., genotyping or nucleic acid sequencing data, and may in some instances be used to identify correlations between specific image features and genotyping or nucleic acid sequence data. In some instances, a machine learning model trained, for example, using a combination of image-derived data and nucleic acid sequence data may subsequently be able to detect and identify changes in genetic or genomic traits based purely on the analysis of the input image data.

### Machine Learning Programs

Any of a variety of commercial or open-source program packages, program languages, or platforms known to those of skill in the art may be used to implement the machine learning models of the disclosed methods and systems. Examples include, but are not limited to, Shogun (www.shogun-toolbox.org), Mlpack (www.mlpack.rog), R (r-project.org), Weka (www.cs.waikato.ac.nz/ml/weka/), Python (www.python.org), and/or Matlab (MathWorks, Natick, MA). Additional examples are provided in the examples described below.

### Statistical Data Analysis Techniques

In addition to the use of machine learning models, the disclosed methods and systems may in some instances also comprise the use of statistical data analysis techniques, for example, to process a multi-dimensional image feature data set produced as output from an image processing and/or machine learning model for the purpose of identifying the key components that underlie the observed variation in tissue phenotype within a population of image patches extracted from tissue images. The combination of one or more statistical analysis methods, e.g., principal component analysis (PCA), used alone or in combination with a machine learning model, may thus be used to generate an image patch characterization data set comprising representations of one or more key attributes (e.g., image features) that provide a basis set of parameters for characterizing tissue samples. In some instances, one or more of the key components (or attributes) that comprise the tissue characterization data set may correspond directly to observable tissue phenotypic traits. In some instances, one or more of the key components (or attributes) that comprise the tissue characterization data set may not correspond directly to observable tissue phenotypic traits but rather may comprise some combination of observable tissue phenotypic traits and/or may comprise latent features, i.e., features that are too subtle to be directly visible in the original images. In preferred instances, the tissue characterization data set may be of reduced dimensionality compared to the multi-dimensional image feature data set produced as output from an image processing and/or machine learning model (i.e., it may provide a compressed representation of the complete feature data set), thereby facilitating handling and comparison of image data to other types of experimental data, e.g., that obtained through nucleic acid sequencing methods. In some instances, one or more statistical analysis methods may be used in combination with one or more of the machine learning models described above.

In some embodiments, the basis set of key attributes identified by a statistical and/or machine learning-based analysis may comprise 1 key attribute, 2 key attributes, 3 key attributes, 4 key attributes, 5 key attributes, 6 key attributes, 7 key attributes, 8 key attributes, 9 key attributes, 10 key attributes, 15 key attributes, 20 key attributes, or more.

Any of a variety of suitable statistical analysis methods known to those of skill in the art may be used in performing the disclosed methods. Examples include, but are not limited to, principal component and other eigenvector-based analysis methods, regression analysis, probabilistic graphical models, or any combination thereof.

### Dimensionality Reduction

As indicated above, dimensionality reduction of the image feature data set extracted from feature-extraction image patches facilitates the annotation of image patch data by pathologists in some instances, or the assignment of a cluster-based label to image patch data in other instances. Image features are generated by passing a random selection of image patches through a trained feature extraction model. This feature extraction model can be, e.g., a neural network, that is pre-trained on other visual distributions (e.g. an ImageNet dataset comprising natural images) or a model that was trained for other computational pathology applications. In either case, the original model may be slightly modified to remove some number of final neural network layers (e.g., the final layer). This results in a neural network model that provides an n-dimensional (where n>>1) output that by itself does not provide a final classification result (typically performed by the final layer of the neural network, that in this case has been removed). Instead, this n-dimensional layer provides an embedding of salient, non-human-interpretable, information of any input (e.g. image patch data) that has been passed through the modified feature extraction model. It can be treated as a distillation of the relevant image patch information (e.g., shapes, colors, textures, visual densities and patterns, relationships between constituent parts of the image, etc.) from the raw pixel information, and thus provides an ‘encoding’ of image feature information.

The output provided by the embedding layer, although much more compact in representation than the raw pixel values, is often of a dimensionality that is still too high to be clustered efficiently. For example, the dimensions of the embedded feature data after feature extraction can often be a 1000-dimensional vector, or greater. This makes the clustering process more difficult, with the clustering being assessed and computed in very high-dimensional space. Additionally, individual features within the embedded data may be redundant or non-informative (which will depend on the feature extraction model).

In some instances of the disclosed methods and systems, these issues may be addressed by performing dimensionality reduction on the embedded data. By using a dimensionality reduction model, such as principal components analysis (PCA), the dimensionality of the embedded data can be further reduced to lower dimensional representations (e.g., principal components) that should disproportionately capture the data variance (e.g., the first principal component will explain more of the variance in the data than any subsequent principal component, and the fall-off in relative importance from one component to the next is often exponential). Using PCA, the principal components are also guaranteed to be orthogonal (i.e., they share no redundancies whatsoever). Thus, a dimensionality reduction approach using, e.g., PCA, constitutes a transformation of the input data representation (embedded image feature data) into a different data representation (i.e., that has a different ‘organization’; mathematically, there is a “change in basis”) that is both much more efficient and removes redundancies. This allows one to cluster the image feature data in a much lower-dimensional space, but one that has been purposely “constructed” to provide the relevant information in a more compact representation (rather than a smaller representation that results simply from discarding useful information). Image feature data is clustered more efficiently and effectively, and can also be better visualized (lower dimensions are easier to plot and visualize than higher dimensions). Examples of other suitable dimensionality reduction techniques that may be used include, but are not limited to, linear discriminant analysis (LDA), canonical correlation analysis (CCA), and non-negative matrix factorization (NMF). The advantages of performing dimensionality reduction as part of the data processing workflow include: (i) more efficient processing, (ii) removal of data redundancy (decorrelation), (iii) generation of a more compact data representation without loss of critical information, and (iv) easier visualization of the data.

### Aggregation Methods for Slide-Level Gene Alteration State Classification

As indicated above, in some instances, an aggregation method may be used to generate a slide-level gene alteration state determination based on image patch classification results. Each slide image is tissue-masked, then image patches are extracted from the whole slide image at, e.g., a fixed pixel size. During training of the gene alteration state classifier, all image patches of interest (either all tissue image patches, or tissue image patches belonging to tissue phenotype group(s) of interest) take a slide-level gene state label (e.g., EGFR+). During classification of an unknown tissue sample, the trained gene alteration state classification model may, in some instances, make a prediction for individual image patches, e.g., a probability score having a value within the range of 0 (zero percent chance) to 1 (one hundred percent chance), that the individual image patch exhibits a given gene alteration state for every image patch extracted from a given tissue sample slide. It is ultimately the slide-level prediction of gene alteration state (and its correctness) that matters for practical implementation of the approach, not the image patch-level predictions. Thus, in some instances, individual image patch-level predictions may be aggregated in order to generate a slide-level prediction. For example, one approach is to take the average (mean) of all image patch predictions (or image patch predictions for image patches belonging to the tissue phenotype groups of interest, if applicable), which then results in a final tissue sample slide-level prediction. Any of a variety of aggregation methods may be used including, but not limited to, calculating a mean, median, or mode, calculating a maximum value, a majority vote determination (e.g., determining the number of individual patch predictions having a value below an experimentally defined threshold (e.g., 0.5) versus the number of individual patch predictions having a value above the experimentally defined threshold, with no weight given to patch prediction magnitude beyond linear separation by the threshold), a central tendency measure (e.g. mean, median, etc.) on the interquartile range, a central tendency or voting measure computed only on extreme values (e.g., disregarding samples within a middle range, such as discarding values 0.25≤x≤0.75, and keeping only data outside that prediction score range), and the like.

### Systems & System Performance Metrics

Also disclosed herein are systems designed to implement the disclosed methods. In some instances, the disclosed systems may comprise one or more processors or computer systems, one or more memory devices, and one or more programs, where the one or more programs are stored in the one or more memory devices and contain instructions (or code) which, when executed by one or more processors, cause the system to perform a method for image-based detection of gene alteration state as described elsewhere herein.

In some instances, the disclosed systems may further comprise, e.g., one or more user interface and/or display devices (e.g., monitors), one or more imaging units (e.g., bright-field, dark-field, phase contrast, or differential interference contrast microscopes, fluorescence microscopes, confocal microscopes, confocal fluorescence microscopes, super-resolution optical microscopes, transmission electron microscopes, or scanning electron microscopes), one or more output devices (e.g., printers), one or more computer network interface devices, or any combination thereof.

In some instances, the performance of the disclosed methods and systems may be assessed, e.g., by determining the area under a receiver operating characteristic curve (AUROC) on either a per-image patch basis, or on a per-slide basis after aggregation of individual image patch results. A receiver operating characteristic (ROC) curve is a graphical plot of the classification model's performance as its discrimination threshold is varied. In some instances, the performance of the disclosed methods and systems (on either a per-image patch basis or a per-slide basis) may be characterized by an AUROC value of at least 0.50, at least 0.55, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some instances, the performance of the disclosed methods and systems may be characterized by an AUROC of any value within the range of values described in this paragraph, e.g., an AUROC value of 0.876. In some instances, the performance of the disclosed methods and systems may vary depending on the specific gene alteration state(s) for which the classification models are trained.

In some instances, the performance of the disclosed methods and systems may be assessed, e.g., by evaluating the clinical sensitivity and clinical specificity for correctly determining gene alteration states in pathology images of tissue specimens. In some instances, the clinical sensitivity (i.e., how often the method correctly classifies a tissue specimen as having a given gene alteration state, as calculated from the number of true positive results divided by the sum of true positive and false negative results), may be at least 50%, at least 60%, at least 70%, at least 75%, at least 80%, at least 85%, at least 90%, at least 95%, at least 98%, at least 99%, or at least 99.9%. In some instances, the clinical specificity (i.e., how often the method correctly classifies a tissue specimen as not having a given gene alteration state, as calculated from the number of true negatives divided by the sum of false positives and true negatives) may be at least 50%, at least 60%, at least 70%, at least 75%, at least 80%, at least 85%, at least 90%, at least 95%, at least 98%, at least 99%, or at least 99.9%. In some instances, adjustment of a threshold used to distinguish between positive and negative results may result in tradeoffs between clinical sensitivity and clinical specificity. For example, the threshold may be adjusted to increase clinical sensitivity with a concomitant decrease in clinical specificity, or vice versa.

In some instances, the positive predictive value (PPV) of the disclosed methods and systems (i.e., the percentage of positive results that are true positives as indicated by a reference method) is calculated as the number of true positives divided by the sum of true positives and false positives and may be at least 70%, at least 75%, at least 80%, at least 85%, at least 90%, at least 95%, at least 98%, at least 99%, or at least 99.9%. In some instances, the negative predictive value (NPV) of the disclosed methods and systems (i.e., the percentage of negative results that are true negatives as indicated by a reference method) is calculated from the number of true negatives divided by the sum of false negatives and true negatives and may be at least 70%, at least 75%, at least 80%, at least 85%, at least 90%, at least 95%, at least 98%, at least 99%, or at least 99.9%.

### Processors, Computer Systems, and Distributed Computing Environments

One or more processors may be used to implement the machine learning-based methods and systems disclosed herein. In addition to running the machine learning and/or statistical analysis methods used to implement the disclosed methods, the one or more processors may be used for inputting data, e.g., image patch data, to the machine learning and/or statistical analysis methods, or for outputting a result from the machine learning and/or statistical analysis methods. The one or more processors may comprise a hardware processor such as a central processing unit (CPU), a graphic processing unit (GPU), a general-purpose processing unit, or other computing platform. The processor may be comprised of any of a variety of suitable integrated circuits, microprocessors, logic devices, field programmable gate arrays (FPGAs), and the like. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations.

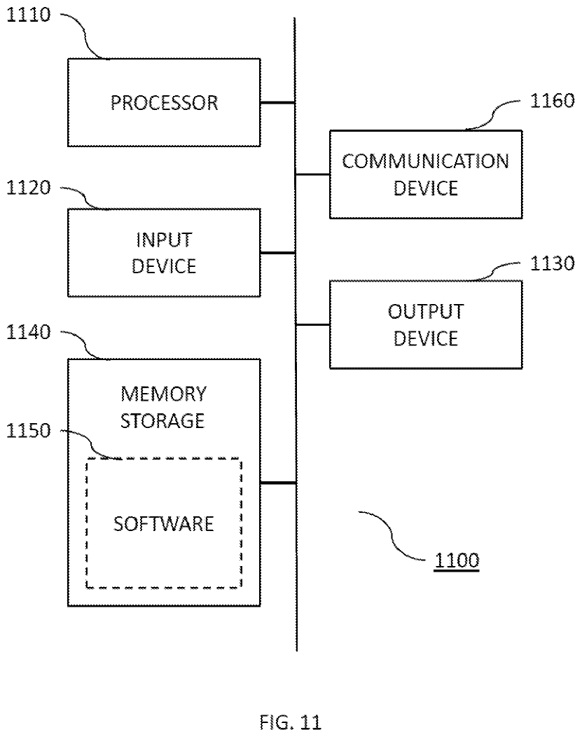

FIG. 11 illustrates an example of a computer system in accordance with one or more examples of the disclosure. Computer system 1100 can be a host computer connected to a network. Computer system 1100 can be a client computer or a server. As shown in FIG. 11, computer system 1100 can be any suitable type of microprocessor-based device, such as a personal computer, workstation, server, or handheld computing device (portable electronic device), such as a phone or tablet. The computer system can include, for example, one or more of processor 1110, input device 1120, output device 1130, storage 1140, and communication device 1160. Input device 1120 and output device 1130 can generally correspond to those described above, and they can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage, such as an electrical, magnetic, or optical memory including a RAM, cache, hard drive, or removable storage disk. Communication device 1160 can include any suitable device capable of transmitting and receiving signals over a network, such as a network interface chip or device. The components of the computer can be connected in any suitable manner, such as via a physical bus or wirelessly.

Software 1150, which can be stored in memory/storage 1140 and executed by processor 1110, can include, for example, the programming that embodies the functionality of the present disclosure (e.g., as embodied in the systems described above).

Software 1150 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1140, that can contain or store programming for use by or in connection with an instruction execution system, apparatus, or device.

Software 1150 can also be propagated within any transport medium for use by or in connection with an instruction execution system, apparatus, or device, such as those described above, that can fetch instructions associated with the program from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Computer system 1100 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer system 1100 can implement any operating system suitable for operating on the network. Software 1150 can be written in any suitable programming language, such as C, C++, Java, or Python. In various embodiments, programs embodying the functionality of the present disclosure can be deployed in different configurations, such as in a client/server arrangement or through a web browser as a web-based application or web service, for example.

Methods and systems of the present disclosure may be implemented by way of one or more machine learning models, e.g., one, two, three, four, five, or more than five machine learning models. An machine learning model can be implemented by way of coded program instructions upon execution by the central processing unit.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1—Predicting NCCN-Guideline Driver Alterations Directly From Lung Adenocarcinoma Whole Slide Images

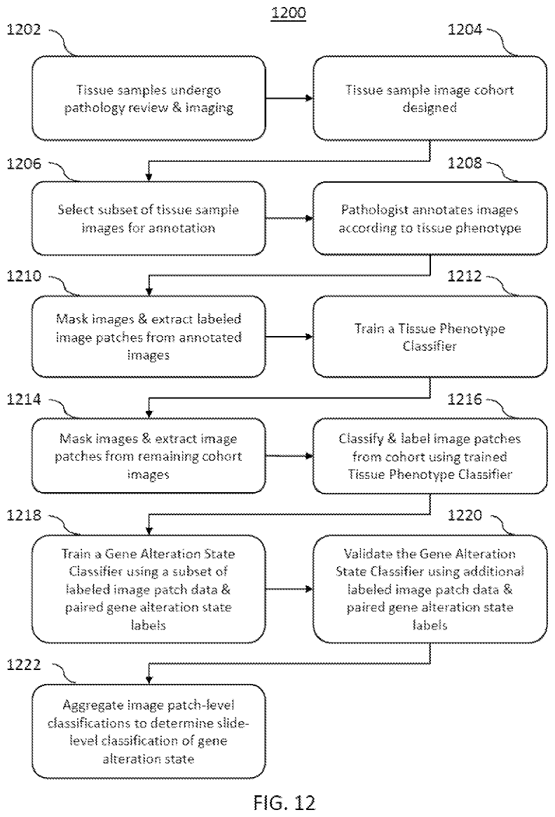

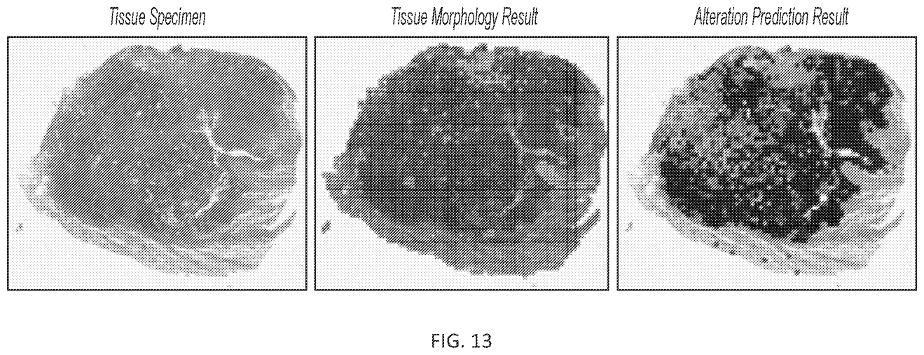

Applicant is currently developing a machine learning-based image analysis process to automatically predict National Comprehensive Cancer Network (NCCN) guideline oncogenic driver mutations directly from whole slide images of lung adenocarcinoma tissue. The training process workflow is illustrated schematically in FIG. 12, and examples of the outputs of convolutional neural network models A (the tissue phenotype classifier) and B (the gene alteration state classifier) are illustrated in FIG. 13.

Referring to FIG. 12, the process begins with cohort selection and transfer of image data. In this non-limiting example, following pathology review, imaging, and transfer of scanned images to a pathology image database (such as Aperio, Leica Biosystems, Inc., Buffalo Grove, IL), 1202, an image cohort for lung adenocarcinoma samples is selected, 1204, each of which has a corresponding sequencing result indicating either the presence of an NCCN-guideline oncogenic driver mutation or the absence of all driver mutations. A subset of the lung adenocarcinoma whole slide tissue images are then selected, 1206, for annotation by a pathologist, 1208, according to tissue phenotype class, e.g., the tissue morphology classes (e.g., tumor, normal, stroma, immune, and necrosis). Image patches are extracted from the slides that have been annotated by the pathologist, 1210, and the labeled image patch data is used to train a convolutional neural network as a tissue phenotype classifier, 1212, configured to classify the extracted tissue image patches into the tissue phenotype classes of interest. The trained convolutional neural network (e.g., the tissue morphology classifier) is used to classify all image patches extracted from the remaining slides of the cohort, 1214, into the classes of interest (e.g., tumor, normal, stroma, immune, necrosis), 1216. Once all image patches have been classified, one may iterate through each morphology class of interest and, using only the labeled image patches from the selected tissue phenotype class and paired gene alteration state data, attempt to train a convolutional neural network—a gene alteration state classifier-that can accurately classify images patches based on the slide-level gene alteration state label, thereby identifying those labeled image patches that are most relevant as indicators of a given gene alteration state. For example, when investigating signal for NCCN driver mutations, tumor image patches appear to be most relevant. Finally, using only the tumor image patches extracted from the cohort slides (including image patches derived from both {gene}+and {gene}−slides), the gene alteration state classifier is trained, 1218, to determine a gene alteration state (e.g., EGFR status in lung adenocarcinoma) at the image patch level. The gene alteration state classifier is validated, 1220, using additional labeled image patch data and paired gene alteration state data. In some instances, the gene alteration state determinations for individual image patches are aggregated, 1222, e.g., using a method like averaging of the individual image patch predictions, to make the final slide level call that is output by the gene alteration state classifier. This method achieves gene alteration state validation performance that is similar to the state-of-the-art performance described in published studies, but does so using an image data set that is far more heterogeneous (and thus expectedly includes more noise) than the data set used in published state-of-the-art results.

Non-limiting examples of machine learning, image processing, and data processing platforms that may be used to implement the method illustrated in FIG. 7, as well as other methods disclosed herein, include Amazon Web Services (AWS) cloud computing services (e.g., using the P2 graphics processing unit (GPU) architecture), TensorFlow (an open-source program library for machine learning), Apache Spark (an open-source, general-purpose distributed computing system used for big data analytics), Databricks (a web-based platform for working with Spark, that provides automated cluster management), Horovod (an open-source framework for distributed deep learning training using TensorFlow, Keras, PyTorch, and Apache MXNet), OpenSlide (a C library that provides an interface for reading whole-slide images), Scikit-Image (a collection of image processing methods), and Pyvips (a Python-based binding for the libvips image processing library).

FIG. 13 provides a non-limiting example of the output of the process illustrated in FIG. 12, with a lung adenocarcinoma tissue specimen image (left), a tissue morphology classification (e.g., tumor, normal, stroma, immune, necrosis) result (middle), and a gene alteration state prediction result (right) obtained by training a gene alteration state classifier using only image patches of interest, e.g., tumor. In the tissue morphology classification image (middle), light grey indicates stroma, intermediate grey indicates normal tissue, darker grey indicates immune cells (visible, e.g., near the top edge of the image slightly left of center), and darkest grey indicates tumor tissue. There is little or no necrotic tissue visible in this example. In the gene alteration state classification image (right), the light grey patches are patches that the model predicts to be strongly associated with the gene alteration state, intermediate grey patches are patches that have no strong association, and the dark grey patches are patches that are strongly associated with a wild type status.

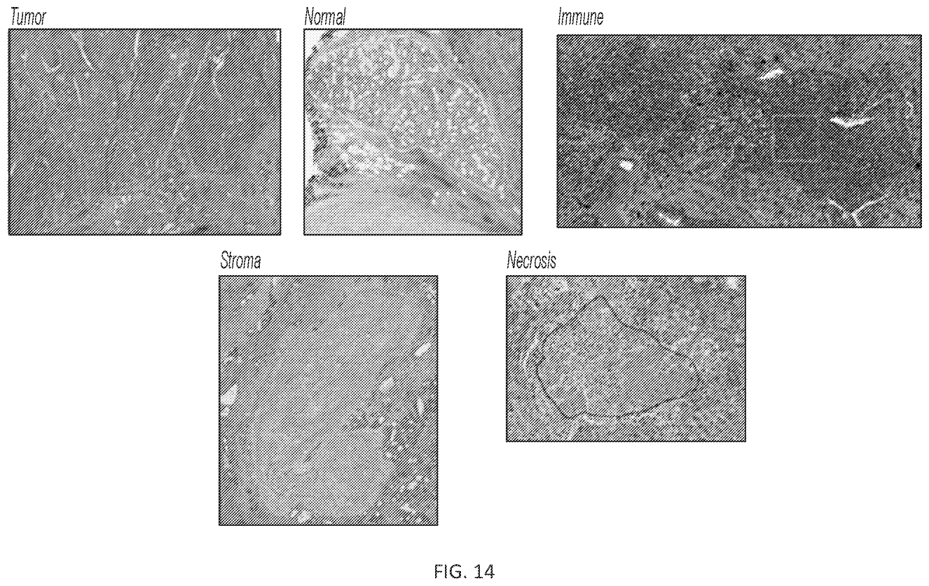

FIG. 14 provides non-limiting examples of tissue images and the corresponding expert annotations (labels) used to train an image patch classifier model to classify image patches extracted from pathology slide images according to tissue phenotype class (e.g., tissue morphology phenotypes).

### Example 2—Predicting NCCN-Guideline Driver Alterations Directly From Lung Adenocarcinoma Whole Slide Images

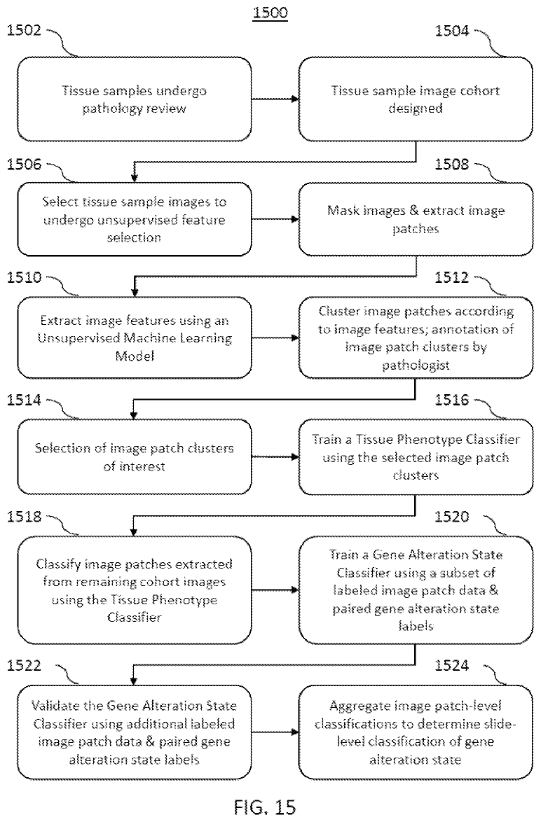

An alternative training process workflow for training a machine learning-based image analysis system to automatically predict National Comprehensive Cancer Network (NCCN) guideline oncogenic driver mutations directly from whole slide images of lung adenocarcinoma tissue is illustrated schematically in FIG. 15.

Referring to FIG. 15, the process 1500 begins with cohort selection and transfer of image data. In this non-limiting example, following pathology review and transfer of scanned images to a pathology image database, 1502, an image cohort for lung adenocarcinoma samples is selected, 1504, each of which has a corresponding sequencing result indicating either the presence of an NCCN-guideline oncogenic driver mutation or the absence of all driver mutations. A subset of the lung adenocarcinoma whole slide tissue images are then randomly selected, 1506, the whole slide images of the subset are masked and tiled, 1508, to extract image patches from the subset of whole slide images, and image features are extracted from the image patches using a machine learning model (e.g., a discriminator sub-network in a generative adversarial network) 1510. The image features extracted from the image patches are then clustered, 1512, to reduce the dimensionality of the image feature data set using, e.g., principal component analysis and k-means clustering, and the image patch data is labeled according to image feature cluster label. In some instances, a pathologist may further annotate the image feature clusters, and this information may be added to the image patch data label. For example, some clusters may be primarily tumor, normal lung, necrosis, stroma, or immune foci. Furthermore, tumor clusters may be further characterized by tumor histological subtype, such as lepidic, acinar, micropapillary, papillary, solid, or mucinous. The labeled image patch data corresponding to tissue phenotype classes of interest, e.g., tissue morphologies (such as tumor, normal, stroma, immune, necrosis, etc.), may then be selected, 1514, and used to train, 1516, a machine learning model (e.g., a convolutional neural network) as a tissue phenotype classifier configured to classify image patches into the tissue phenotype classes of interest. The trained tissue phenotype classifier is then used to classify tissue image patches derived by masking and extracting image patches, 1518, from all remaining whole slide pathology images of the cohort into their respective tissue phenotype classes, e.g. tissue morphological classes. One may then iterate through each tissue phenotype class of interest, e.g., using only tumor-associated image patches along with paired gene alteration state data, and attempt to train another machine learning model (e.g., a convolutional neural network model) as a gene alteration state classifier. Iteration through image patches assigned to selected tissue phenotype classes of interest allows one to identify those tissue image patch categories that are most highly correlated (i.e., provide a signal or indicator of) a given gene alteration state. Finally, using only the subset of labeled image patch data belonging to the signal-containing classes (i.e., the labeled image patch data that is most highly correlated with a given gene alteration state, e.g. tumor and stroma patches in some instances), one may further train, 1520, tune, validate, 1522, and deploy the gene alteration state classifier for use in determining gene alteration state at the slide level from analysis of pathology images of a tissue specimen. Several approaches are possible for determining a slide level gene alteration state, for example, one may allow all sub-selected image patches to inherit the specimen (slide) level gene alteration state label, make a gene alteration state determination for each image patch, and then aggregate the patch-level determinations, 1524, e.g., by averaging the patch-level determinations to make the slide level call. Another approach is to use the sub-selected image patches (e.g., the subset of image patches that are the best indicator(s) for the gene alteration state) and create neural network “embeddings” (e.g., a method used to represent discrete variables as continuous vectors), e.g., using another feature extractor model, aggregate the embeddings in some fashion (e.g., by averaging features for the image patch embeddings for an entire slide image), and train the gene alteration state classification model using this slide-level representation of the image data and the corresponding sequencing-based gene alteration labels. In some instances, the output of the gene alteration state classifier may be a binary determination (i.e., a yes or no determination) of whether a specific gene alteration state is present in the tissue sample. In some instances, the output of the gene alteration state classifier may be a determination of whether the tissue sample exhibits one or more of a plurality of potential gene alteration states.

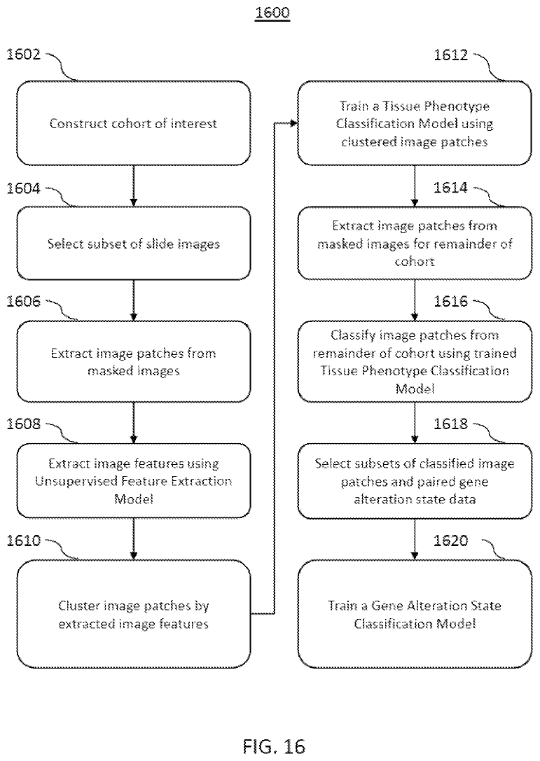

FIG. 16 provides a schematic illustration of a simplified training process workflow 1600 that utilizes an unsupervised feature extraction model. In a first step, 1602, a cohort of pathology tissue sample images for a disease state of interest are selected, and the images for a subset of the cohort, 1604, are processed, 1606, to extract image patches—“feature extraction patches”—which will be used as input data for training an unsupervised machine learning model, 1608, as a “feature extraction model”. The feature extraction patches are then clustered, 1610, according to the image features identified by the model to create a cluster-labeled image patch data set. Subsequently, the cluster-labeled image patch data is used to train a machine learning model (e.g., a convolutional neural network) as a tissue phenotype classification model, 1612. All remaining pathology slide images in the cohort are processed to extract image patches, 1614, which are then classified into image patch clusters (which may or may not correspond directly to tissue morphology classes as identified by a pathologist) by the trained tissue phenotype classification model, 1616. Selected subsets of the clustered image patch data generated by the tissue phenotype classification model, 1618, are used in combination with corresponding gene alteration state labels obtained from, e.g., next generation sequencing data, are used to train another machine learning model (e.g., a convolutional neural network) to function as a gene alteration state classifier, 1620, which maps labeled image patch data (e.g., comprising an image feature cluster label) as input to a determination of gene alteration state in the tissue sample as output.

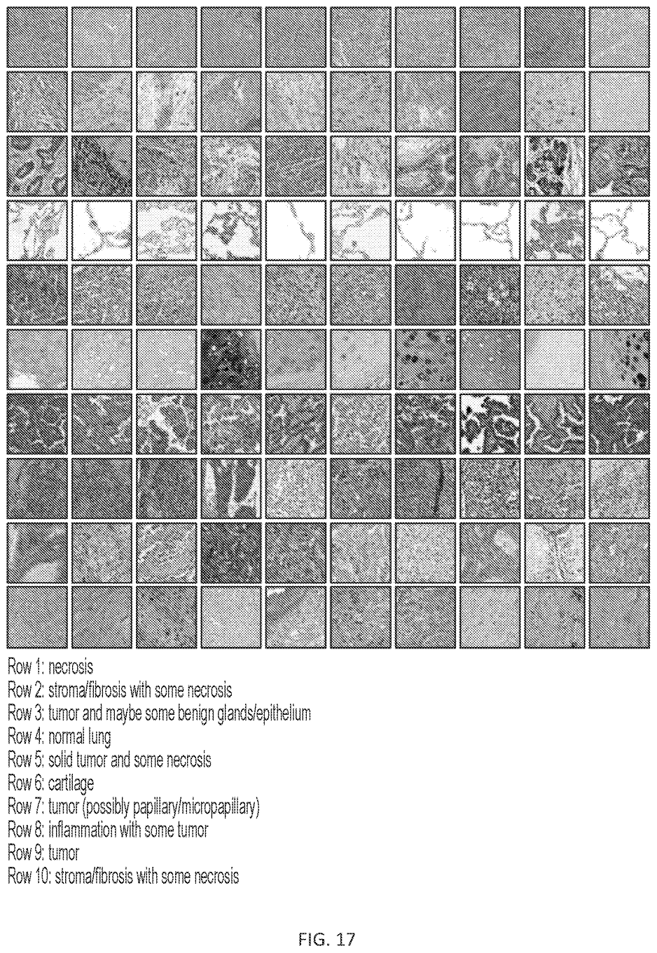

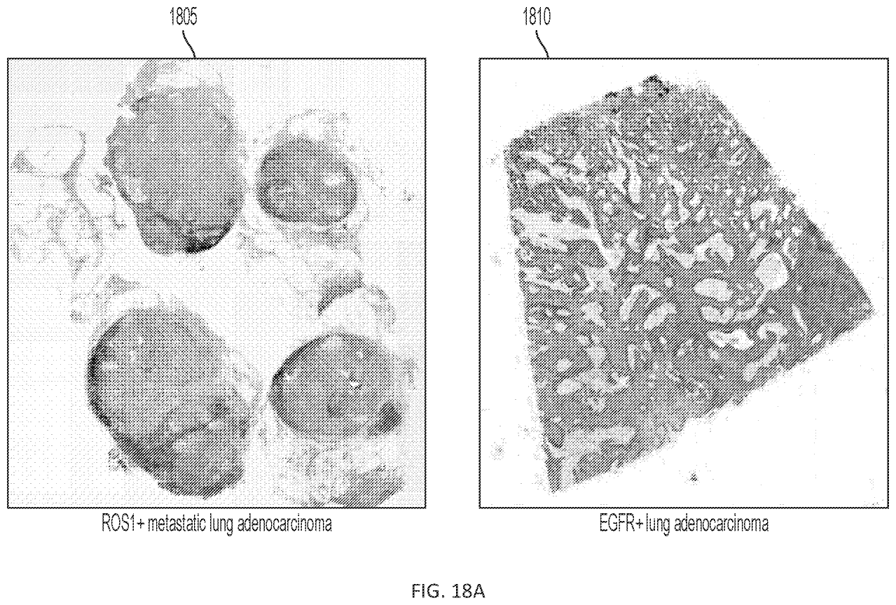

FIG. 17 provides a non-limiting example of images and image feature clusters from unsupervised learning on feature vectors from deep neural networks that have been pre-trained on the ImageNet dataset. Each row represents a different cluster and contains 10 examples of image patches whose latent features belong to that cluster. The pathology assessments of representative image patches in each cluster are listed below the images.

### Example 3—Gene Fusion Prediction in Lung Adenocarcinoma

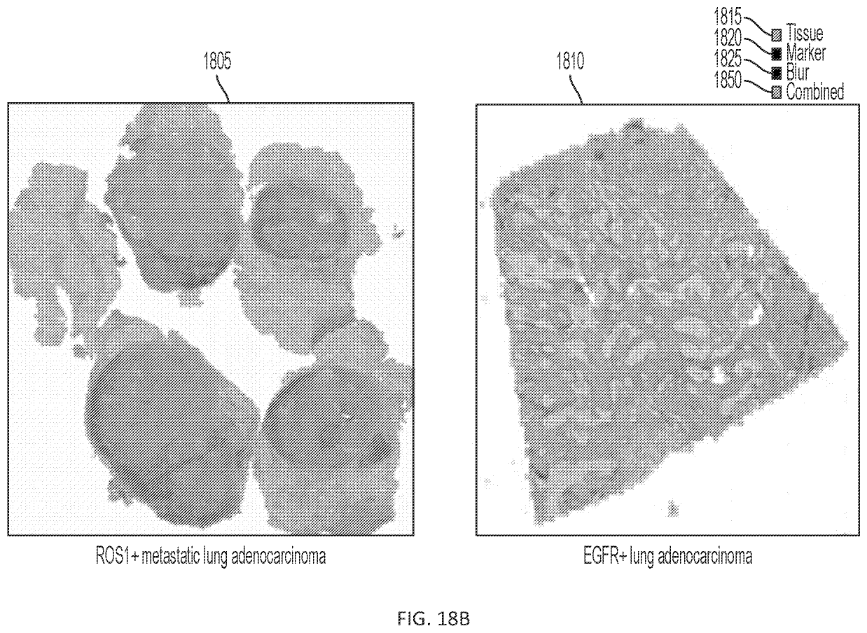

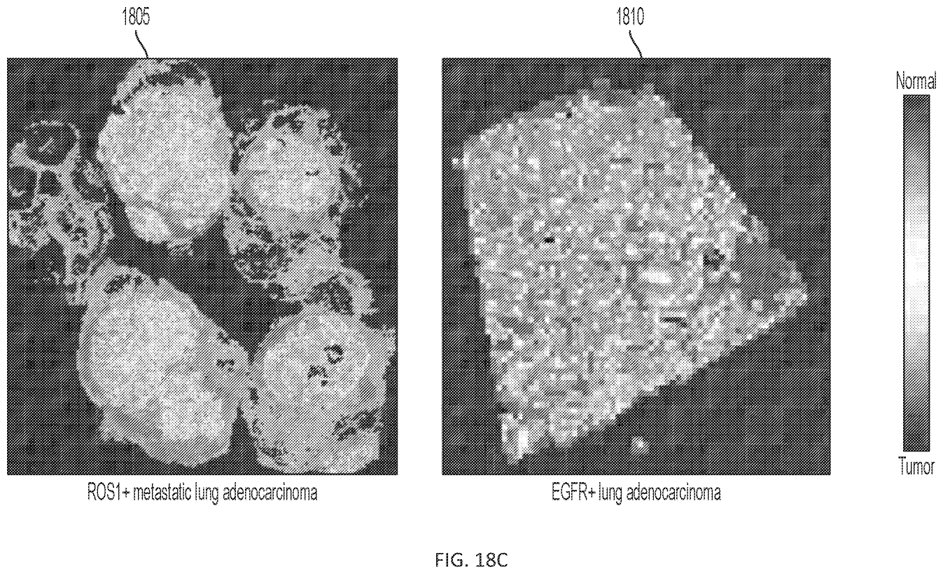

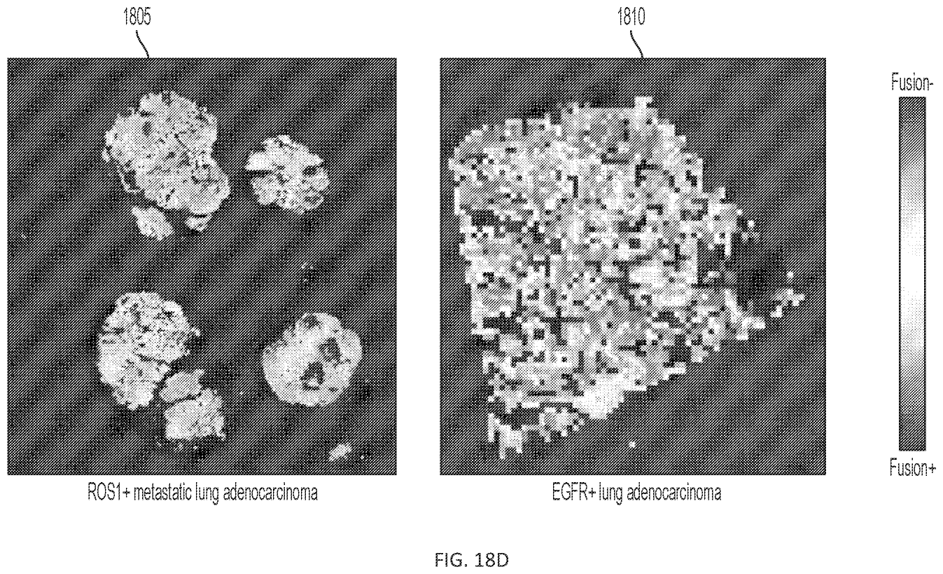

FIGS. 18A-18D illustrate actionable fusion prediction in lung adenocarcinoma. FIG. 18A provides a non-limiting example of pathology slide images for lung adenocarcinoma. The left image 1805 is a slide for metastatic lung adenocarcinoma with an ROS1 fusion. The right image 1810 is a slide for lung adenocarcinoma with an EGFR mutation. FIG. 18B provides a non-limiting example of results from quality control. As illustrated in FIG. 18B, the quality control process may identify tissue 1815, marker 1820, blur 1825, and combined image features 550. FIG. 18C provides a non-limiting example of tumor region detection. As illustrated in FIG. 18C, the darker the region, the more likely it is a tumor region. FIG. 18D provides a non-limiting example of prediction of fusion status. As illustrated in FIG. 18D, the darker the region, the more likely it comprises a gene fusion.

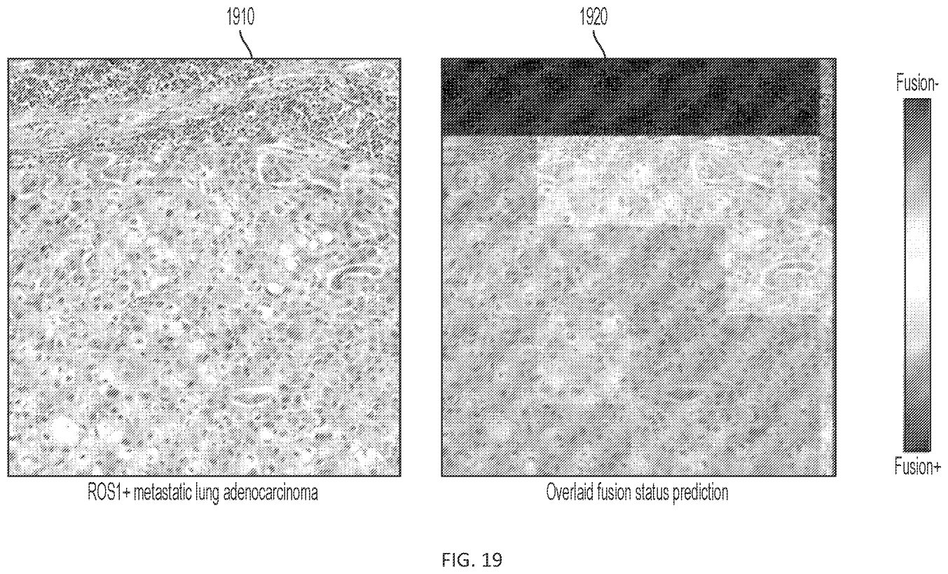

FIG. 19 provides a non-limiting example of the prediction of ROS1 gene fusion status. The images shown in FIG. 19 are examples of the final output that may be provided to the pathologist. The left image 1910 indicates a fusion positive slide for metastatic lung adenocarcinoma comprising a ROS1 fusion. The right image 1920 indicates the same field of view from image 1910 with an overlaid heatmap of gene fusion prediction. When comparing the two images, one can see how the tumor detection algorithm rejected the image patches containing no tumor. In addition, confidence metric(s) for the prediction (as depicted by the intensity of the heatmap) may vary across the tumor area. Confidence metrics may be highest in areas with signet ring cells. As can be seen, the digital pathology image processing system 110 may provide output in formats that make clear to the pathologist that the digital pathology model is based on interpretable morphologic features.

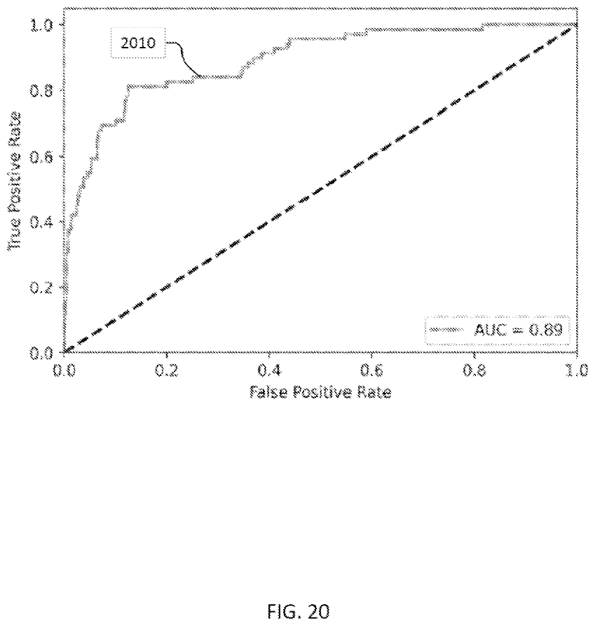

Experiments on actionable fusion prediction in lung adenocarcinoma were conducted to validate the digital pathology model and methods disclosed herein. FIG. 20 provides a non-limiting example of a receiver operating characteristic (ROC) curve 2010 for image patch-based gene fusion prediction. The training set for the digital pathology model comprised 270 resections. 18.5% of them were fusion positive, i.e., 50 slides derived from 5 patients. Among these fusion positive slides, 5 slides were ALK fusion positive and 45 slides were ROS1 fusion positive. The test set comprised 598 resections and biopsies. 11% of them were fusion positive, i.e., 68 slides. Among these fusion positive slides, 8 slides were NTRK fusion positive and 60 slides were ROS1 fusion positive. For a cut-off set at 0.5, the performance statistics were as follows: the positive predictive value (PPV) was 0.46 and negative predictive value (NPV) was 0.97, with an overall area under the curve (AUC) of 0.89.

### Example 4—Networked Computing Systems for Digital Pathology

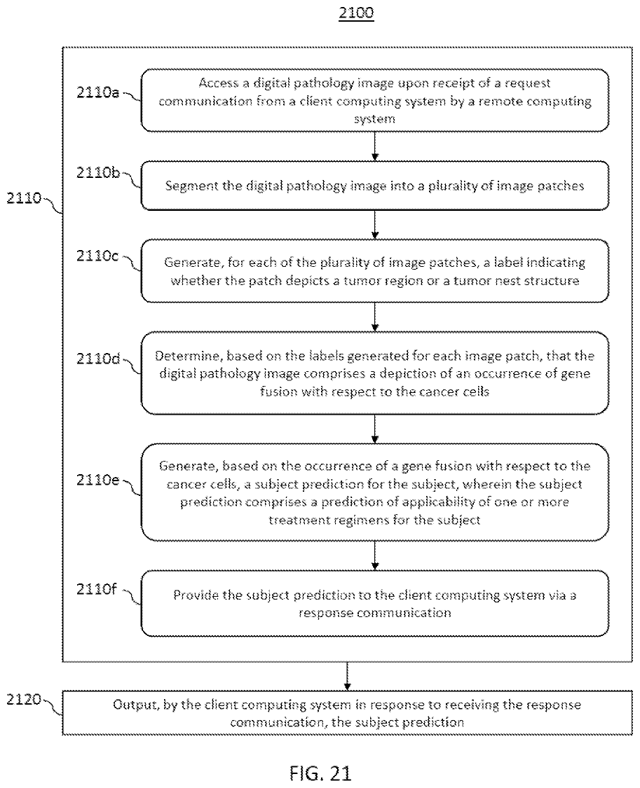

FIG. 21 illustrates an example method 2100 for enabling end users to request subject predictions based on processing of digital pathology images. The method may begin at step 2110, where the digital pathology image generation system 120 depicted in FIG. 1 may transmit, from a client computing system to a remote computing system, a request communication to process a digital pathology image that depicts cancer cells in a particular section of a biological sample from a subject, where in response to receiving the request communication from the client computing system, the remote computing system performs operations comprising the following sub-steps. At sub-step 2110a, the remote computing system may access the digital pathology image. At sub-step 2110b, the remote computing system may segment the digital pathology image into a plurality of image patches. At sub-step 2110c, the remote computing system may generate, for each of the plurality of image patches, a label indicating whether the patch depicts, e.g., a tumor region or a tumor nest structure. At sub-step 2110d, the remote computing system may determine, based on the labels generated for each image patch, that the digital pathology image comprises a depiction of an occurrence of gene fusion with respect to the cancer cells. At sub-step 2110e, the remote computing system may generate, based on the occurrence of gene fusion with respect to the cancer cells, a subject prediction for the subject, wherein the subject prediction comprises a prediction of applicability of one or more treatment regimens for the subject. At sub-step 2110f, the remote computing system may provide the subject prediction to the client computing system via a response communication. At step 2120, the client computing system may output, in response to receiving the response communication, the subject prediction. In some instances, one or more steps of the method depicted in FIG. 21, may be repeated where appropriate. Although this disclosure describes and illustrates particular steps of the method of FIG. 21 as occurring in a particular order, this disclosure contemplates any suitable steps of the method of FIG. 21 as occurring in any suitable order. Moreover, although this disclosure describes and illustrates an example method for enabling end users to request subject predictions, including the particular steps of the method depicted in FIG. 21, this disclosure contemplates any suitable method for enabling end users to request subject predictions, including any suitable steps, which may include all, some, or none of the steps of the method depicted in FIG. 21, where appropriate. Furthermore, although this disclosure describes and illustrates particular components, devices, or systems carrying out particular steps of the method of FIG. 21, this disclosure contemplates any suitable combination of any suitable components, devices, or systems carrying out any suitable steps of the method of FIG. 21.

### Example 5—Method for Determining an Absence of Gene Fusion

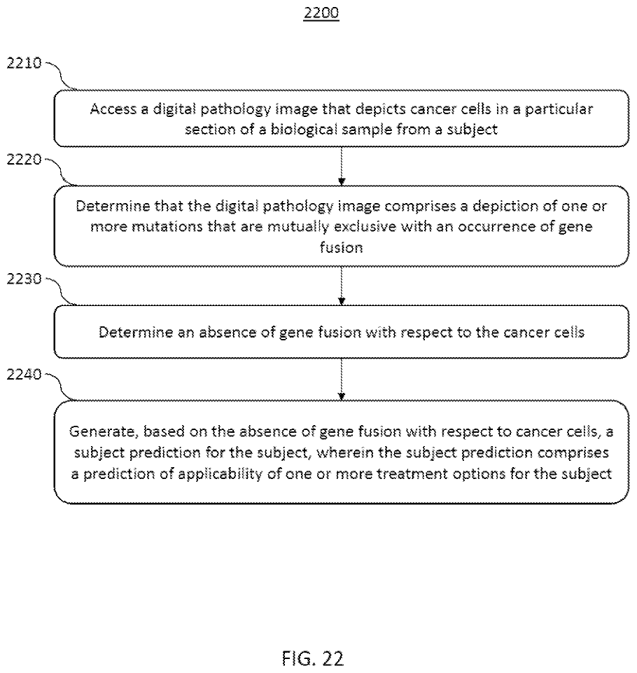

FIG. 22 illustrates an example method 2200 for identifying a lack of gene fusion with respect to a set of detected cancer cells. The method may begin at step 2210, where the digital pathology image processing system 110 shown in FIG. 1 may access a digital pathology image that depicts cancer cells in a particular section of a biological sample from a subject. At step 2220, the digital pathology image processing system 110 may determine that the digital pathology image comprises a depiction of one or more mutations that are mutually exclusive with an occurrence of gene fusion. At step 2230, the digital pathology image processing system 110 may determine an absence of gene fusion with respect to the cancer cells. At step 2240, the digital pathology image processing system 110 may generate, based on the absence of gene fusion with respect to the cancer cells, a subject prediction for the subject, wherein the subject prediction comprises a prediction of applicability of one or more treatment regimens for the subject. In some instances, one or more steps of the method of FIG. 22 may be repeated, where appropriate. Although this disclosure describes and illustrates particular steps of the method of FIG. 22 as occurring in a particular order, this disclosure contemplates any suitable steps of the method of FIG. 22 occurring in any suitable order. Moreover, although this disclosure describes and illustrates an example method for identifying a lack (or an absence) of gene fusion, including the particular steps of the method of FIG. 22, this disclosure contemplates any suitable method for ruling out gene fusion, including any suitable steps, which may include all, some, or none of the steps of the method of FIG. 22, where appropriate. Furthermore, although this disclosure describes and illustrates particular components, devices, or systems carrying out particular steps of the method of FIG. 22, this disclosure contemplates any suitable combination of any suitable components, devices, or systems carrying out any suitable steps of the method of FIG. 22.

While preferred embodiments of the disclosed methods and systems have been illustrated and described herein, it will be obvious to those skilled in the art that such embodiments are provided by way of example only. Numerous variations, changes, and substitutions will now occur to those skilled in the art without departing from the inventive concepts described herein. It should be understood that various alternatives to the embodiments described herein may be employed in any combination in practicing the disclosed methods and systems. It is intended that the following claims define the scope of the invention and that methods and systems within the scope of these claims, and their equivalents, be covered thereby.

1. A method for determining a gene alteration state in a tissue sample comprising:
inputting, using one or more processors, a plurality of image patches derived from one or more pathology images of the tissue sample into a tissue phenotype classification model, the tissue phenotype classification model configured to classify image patches into a tissue phenotype class;
classifying, using the one or more processors and the tissue phenotype classification model, the image patches to generate a labeled image patch data set for the tissue sample;
inputting, using the one or more processors, the labeled image patch data set into a gene alteration state classification model, the gene alteration state classification model configured to determine a gene alteration state for the tissue sample based on the labeled image patch data set; and
outputting, using the one or more processors and the gene alteration state classification model, the gene alteration state for the tissue sample.

2. The method of claim 1, wherein the gene alteration state classification model is configured to determine a gene alteration state for one or more genes in the tissue sample based on the labeled image patch data.

3. The method of claim 1, wherein the tissue phenotype classification model is trained using a plurality of tissue phenotype classification model training image patches, and wherein each tissue phenotype classification model training image patch is labeled with a tissue phenotype class selected from a plurality of tissue phenotype classes.

4. The method of claim 3, wherein the tissue phenotype classification model training image patches are derived from pathology images of tissue samples from the same tissue type.

5. The method of claim 4, wherein the tissue phenotype classification model training image patches are manually labeled with the tissue phenotype class.

6. The method of claim 3, wherein the tissue phenotype classification model training image patches are labeled using a clustering process, the clustering process comprising extracting image features from the tissue phenotype classification model training image patches, and clustering the tissue phenotype classification model training image patches based on the extracted image features.

7. The method of claim 6, wherein labels are assigned to the tissue phenotype classification model training image patches based on the extracted image feature clusters.

8. The method of claim 6, wherein the image features are extracted from the tissue phenotype classification model training image patches using a pre-trained image feature extraction model.

9-10. (canceled)

11. The method of claim 6, wherein the image features are extracted from the tissue phenotype classification model training image patches using an unsupervised image feature extraction model.

12-13. (canceled)

14. The method of claim 6, further comprising performing a dimensionality reduction on the extracted image features prior to clustering the tissue phenotype classification model training image patches based on a reduced representation of the extracted image features.

15. The method of claim 1, wherein the gene alteration state classification model is trained using a plurality of gene alteration state classification model training image patches, and wherein each gene alteration state classification model training image patch is labeled with a tissue phenotype class and a gene alteration state.

16-20. (canceled)

21. The method of claim 1, wherein an output of the gene alteration state classification model is a determination of the presence or absence of a mutation in at least one gene in the tissue sample.

22. The method of claim 1, wherein an output of the gene alteration state classification model is a probability that the tissue sample has a mutation in at least one gene.

23. The method of claim 1, wherein an output of the gene alteration state classification model is a probability that the tissue sample does not have the mutation in at least one gene.

24-25. (canceled)

26. The method of claim 3, wherein the plurality of tissue phenotype classes comprises one or more tumor phenotype classes, one or more normal phenotype classes, one or more stroma phenotype classes, one or more immune phenotype classes, one or more necrosis phenotype classes, or any combination thereof.

27-29. (canceled)

30. The method of claim 1, wherein the one or more pathology images are images of a cancerous tissue sample.

31-73. (canceled)

74. A method for selecting a treatment for an individual having cancer, the method comprising:
determining a gene alteration state of a gene of interest in a tissue sample from the individual using the method of claim 1; and
selecting a treatment based on the determined gene alteration state.

75-77. (canceled)

78. A method of treating an individual having cancer comprising selecting a treatment for the individual using the method of claim 1; and administering the treatment to the individual.

79-90. (canceled)

91. A method for determining a gene alteration state in a tissue sample comprising:
inputting, using one or more processors, a plurality of image patches derived from one or more pathology images of the tissue sample into a tissue phenotype classification model, the tissue phenotype classification model configured to classify image patches into a tissue phenotype class; and
classifying, using the one or more processors and the tissue phenotype classification model, the image patches to generate a labeled image patch data set for the tissue sample by:
extracting image features from the image patches of the plurality of image patches;
clustering the image patches of the plurality of image patches based on the extracted image features; and
labeling the image patches of the plurality of image patches based on an associated image patch cluster.

92-186. (canceled)

187. A method of treating an individual having a disease, the method comprising:
inputting, using one or more processors, a plurality of image patches derived from one or more pathology images of a tissue sample from the individual into a tissue phenotype classification model, the tissue phenotype classification model configured to classify image patches into a tissue phenotype class;
classifying, using the one or more processors and the tissue phenotype classification model, the image patches to generate a labeled image patch data set for the tissue sample;
inputting, using the one or more processors, the labeled image patch data set into a gene alteration state classification model, the gene alteration state classification model configured to determine a gene alteration state for the tissue sample based on the labeled image patch data set;
determining, using the one or more processors, a disease type of the disease of the individual based on the gene alteration state determined for the tissue sample;
based on the determined disease type, determining, using the one or more processors, a therapy for treating the disease type; and
based on the determined therapy, treating the individual with the therapy.

188-224. (canceled)
